(19) United States
(12) Patent Application Publication
(10) Pub. No.: US 2022/0113313 A1
(43) Pub. Date: Apr. 14, 2022

(54) SYSTEMS AND METHODS FOR VESICLE CARGO LABELING AND DETECTION

(71) Applicant: NanoView Biosciences, Inc., Brighton, MA (US)

(72) Inventors: George G. Daaboul, Watertown, MA (US); Veronica Sanchez, Newton, MA (US); Aditya Dhande, Malden, MA (US)

(21) Appl. No.: 17/422,353

(22) PCT Filed: Jan. 31, 2020
(86) PCT No.: PCT/US2020/016098

§ 371 Date: Jul. 12, 2021

Related U.S. Application Data
(60) Provisional application No. 62/800,110, filed on Feb. 1, 2019.

(51) Int. Cl. G01N 33/574; G01N 33/58

(52) U.S. Cl. CPC G01N 33/57488; G01N 33/582

(57) ABSTRACT

Presented herein are systems and methods for detecting biomolecular cargo [e.g., protein, e.g., DNA, e.g., RNA (e.g., microRNA, e.g., non-coding RNA), e.g., dyes, e.g., aptamers] inside of particles (e.g., exosomes, e.g., viruses, e.g., extracellular vesicles) contained in complex biological samples (e.g., cells, e.g., tissues, e.g., human blood, plasma, and/or serum).

### CROSS REFERENCE TO RELATED APPLICATIONS

This application claims priority to and benefit of U.S. Application Ser. No. 62/800,110, filed on Feb. 1, 2019, the contents of which are incorporated by reference in their entirety.

### TECHNICAL FIELD

The present invention relates generally to the detection of molecular cargo (e.g., biomolecules, e.g., biomolecules associated with cancer, e.g., pancreatic cancer, e.g., biomolecules such as protein and DNA) inside of particles (e.g., nanoparticles, e.g., exosomes, e.g., vesicles such as viruses or extracellular vesicles).

### BACKGROUND

The ability to detect biological target molecules is fundamental to our understanding of both cell physiology and disease progression, as well as for use in various applications such as early and rapid evaluation, e.g., diagnosis of, disease. Fluorescent microscopy is used in biochemistry and other life science fields to analyze biological molecules (e.g., DNA, RNA, protein). Current methods localize biological molecules within cells and tissues with specific antibodies using fixation and permeabilization techniques. Such procedures allow one not only to detect well-characterized cellular structures but also to provide information about any detectable molecules within the cells and tissues.

However, these cell-based techniques are limited by factors such as cellular membrane complexity, complexes of proteins within the cellular membrane, and cellular organelles. In particular, these conventional techniques require high concentrations of and/or exposure to cellular fixation and/or permeabilization reagents that may affect and even damage biomolecular structure and/or function (e.g., antigen presentation and/or function on cells that are used to detect/monitor disease progression). The heterogeneity of cellular and tissue samples often limit detection sensitivity and accuracy required for accurate disease diagnosis, staging, and/or monitoring.

Therefore, there is a need for systems and methods for detecting nanomolecular cargo inside of biological samples with high sensitivity and specificity for the diagnosis, staging, or determination of risk of disease in a subject.

### SUMMARY

Presented herein are systems and methods for detecting biomolecular cargo [e.g., protein, e.g., DNA, e.g., RNA (e.g., microRNA, e.g., non-coding RNA), e.g., dyes, e.g., aptamers] inside of particles (e.g., exosomes, e.g., viruses, e.g., virus-like particles, e.g., vesicles, e.g., extracellular vesicles (EVs)) contained in complex biological samples (e.g., cells, e.g., tissues, e.g., human blood, plasma, and/or serum).

The technology described herein allows a user to analyze heterogeneous populations of vesicles derived from complex biological samples (such as human cells and tissues) that may help characterize, diagnosis, and/or stage disease (e.g., cancer, e.g., neural diseases such as Alzheimer's). For example, the described systems and methods can differentiate between vesicles such as EVs that contain biomolecular cargo characteristic of disease state and/or progression, further increasing sensitivity and accuracy of disease detection, staging, and/or monitoring. In addition, the described technology can be used to monitor therapeutic treatment regimens. For example, the technology can be used to assess whether therapeutic engineered vesicles (e.g., stem-cell derived EVs engineered to deliver therapeutic agents) that are delivered to diseased tissue improve disease prognosis and/or staging (e.g., for regenerative medicine).

Overexposure to fixation and permeabilization reagents can damage antigens that are used to detect and/or monitor disease. In contrast to conventional cell-based assays, the described technology uses a combination of unique fixation and permeabilization protocols that require less exposure time to and/or less concentration of potentially toxic chemical agents that can negatively impact biological structure and function. Moreover, the described systems and methods detect molecular cargo within and/or outside of EVs that contain similar (or identical) biological information to its parental cell.

For example, conventional cell-based techniques use fixing protocol that requires 4% PFA and incubation times from 1 hour to overnight (e.g., 16 hours at 4 degrees C.). Incubation times over 1 hour can damage antigens. In contrast to conventional cell-based techniques, the described technology can effectively detect cargo using 2% PFA with incubation times under 1 hour. In addition, cell and tissue permeabilization protocols require from 0.05% to 1% concentration of Triton™-X or Tween® at incubation times from about 30 minutes to 5 hours. In contrast, the described technology can effectively detect cargo using less than 0.5% Triton™-X and 1 hour incubation times.

For cargo imaging and/or detection, fixation of vesicles followed by permeabilization of the membrane of the vesicles allows access to the luminal and internal molecular cargo. Fixation crosslinks components of the vesicle, making the vesicle more rigid and resistant to losing its structure when permeabilized. Optimization of the fixing and permeabilization parameters for vesicles is difficult. Immobilizing vesicles onto a solid phase surface (e.g., a substrate described herein) followed by fixing improves the stability of the structure when permeabilized. In detection methodologies that do not immobilize vesicles on a substrate, but instead perform detection in liquid phase (e.g., flow cytometry), vesicles that are fixed and permeabilized do not maintain their structure. The vesicles will, as a result, solubilize. Solubilization will result in detection of molecular components as individual entities, and not as a collection of molecules that can be identified as belonging to and/or associated with individual vesicles. Accordingly, in certain embodiments, by providing for sensitive detection of vesicles and their biomolecular cargo, when immobilized on solid substrates, techniques described herein allow for particular biomolecular cargo to be identified as belonging to particular vesicles.

Sensitive detection of molecular cargo within vesicles allows therapeutic development. For example, the described technology can be used to analyze molecular cargo within stem-cell derived vesicles to help develop natural therapeutic agents in regenerative medicine. Moreover, once active cargo can be determined, vesicles can be engineered to package certain therapeutic cargo (e.g., RNA, e.g., microRNA) for therapeutic treatment.

In addition, the described technology serves as a diagnostic and treatment monitoring platform. Vesicles such as EVs carry information from the parental cell, which is highly informative to disease detection and/or staging. Surface markers of the vesicles can reveal what is going on within the cell and/or tumor site. For example, an EV derived from a tumor site can include a population of proteins from the tumor site. In this scenario, the EVs would carry a marker indicative of the tumor and allow a user to test for the disease without having to do a complex biopsy.

The described technology also provides advantages in increased sensitivity compared to conventional cell-based fluorescence assays. In certain embodiments, the described technology uses optical substrates for enhanced detection of molecular cargo. The ability of the described optical substrates to generate enhanced fluorescence signals provides for increased sensitivity and detection of EVs that may facilitate disease detection and/or monitoring, and other clinical applications. In certain embodiments, the ability of the described systems to simultaneously co-localize both enhanced contrast and fluorescence signals provides for imaging both the outside and inside of particles.

In one aspect, the invention is directed to a method of isolating, labeling, and imaging vesicles [e.g., nanovesicles; e.g., extracellular vesicles (e.g., exosomes); e.g., viruses; e.g., virus-like particles. e.g., liposomes] and their biomolecular cargo [e.g., one or more biomolecules of interest potentially present within at least a portion of the vesicles], the method comprising: (a) contacting a top surface of a substrate (e.g., a substantially planar reflective substrate) with a sample comprising the vesicles [e.g., a sample obtained from a subject; e.g., a complex biological sample, such as blood, plasma, saliva, etc.; e.g., a processed sample (e.g., comprising vesicles in a buffer)], thereby capturing one or more vesicles present in the sample [e.g., wherein the surface of the substrate comprises one or more capture agents (e.g., antibodies), each capture agent specific to a particular target agent (e.g., antigen) associated with (e.g., expressed on a surface of) at least a portion of the vesicles]; (b) [e.g., following step (a) (e.g., so as to permeabilize the vesicles after capture); e.g., before step (a) (e.g., so as to permeabilize the vesicles prior to capture, e.g., in-solution)] contacting vesicles with a permeabilization solution comprising a permeabilization agent [e.g., an organic solvent (e.g., methanol, acetone, etc.); e.g., a detergent (e.g., Triton™-X, Tween®, NP-40 etc.); e.g., a selective detergent (e.g., that interacts with cholesterol specifically, such as saponin, digitonin, leucopem, etc.)], thereby permeabilizing the vesicles; (c) following step (b), contacting vesicles with one or more fluorescent cargo labels {e.g., a protein (e.g., antibody), a nucleic acid [e.g., an DNA oligonucleotide (e.g., a DNA aptamer), an RNA oligonucleotide (e.g., an RNA aptamer)], a peptide, a dye, etc.}, wherein each fluorescent cargo label is: (i) specific to a particular biomolecule of interest of one or more biomolecules of interest (e.g., and potentially present in at least a portion of the vesicles) and (ii) comprises a particular fluorescent species, thereby labeling the biomolecular cargo (e.g., the one or more biomolecules of interest) within the vesicles; (d) directing excitation light toward the top surface of the substrate, thereby exciting the one or more fluorescent cargo labels with which the vesicles are labeled; (e) detecting, with one or more detectors, fluorescent light emitted from the one or more fluorescent cargo labels as a result of excitation by the excitation light [e.g., imaging the top surface of the substrate at one or more fluorescent wavelengths, each corresponding to an emission wavelength of (a fluorescent species of) a fluorescent cargo label, thereby obtaining one or more fluorescent images, each associated with a particular fluorescent cargo label and biomolecule of interest to which the particular fluorescent cargo label is specific]; and (g) using the detected fluorescent light to detect and/or quantify, at least a portion of the one or more biomolecules of interest present within the vesicles.

In certain embodiments, the method comprises performing step (b) and (c) following step (a), so as to permeabilize and label the vesicles after they are captured onto the top surface of the substrate.

In certain embodiments, comprising performing step (b) and step (c) before step (a), so as to permeabilize and label the vesicles before they are captured onto the top surface of the substrate.

In certain embodiments, the vesicles are less than or approximately equal to 1 micron in diameter (e.g., about 500 nm or less in diameter or less; about 200 nm or less in diameter; about 150 nm or less in diameter) (e.g., ranging from about 10 nm to about 150 nm in diameter; e.g., ranging from about 30 to about 100 nm in diameter).

In certain embodiments, the vesicles are extracellular vesicles. In certain embodiments, the extracellular vesicles are exosomes.

In certain embodiments, the method comprises [e.g., following step (a) and/or before step (b) (e.g., so as to fix (crosslink) the vesicles after capture); e.g., after step (a) and/or before step (b) (e.g., so as to fix (crosslink) the vesicles prior to capture (e.g., in-solution))] contacting the vesicles with a crosslinking agent [e.g., paraformaldehyde (PFA), gluteraldehyde, DENT's fixative (e.g., methanol with BSA), and the like] [e.g., wherein a concentration of the cross-linking agent is about 4% or less (e.g., about 2% or less)], thereby fixing (crosslinking) the vesicles (e.g., wherein the crosslinking agent does not require a dehydration step)[e.g., other fixatives include, e.g., alcohols (e.g., methanol, ethanol), acetone, formaldehyde, formalin].

In certain embodiments, the method comprises incubating the vesicles with the crosslinking agent for a duration selected to avoid over-fixation of the vesicles {e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the duration is selected based on a size of the vesicles; e.g., wherein the duration is less than or approximately equal to 1 hour (e.g., about 30 minutes or less; about 10 minutes or less); e.g., and subsequently washing the surface of the substrate [e.g., with buffer (e.g., HEPES buffer, water, PBS)], e.g., to remove excess crosslinking agent and preserve the one or more target agents present on a surface of the vesicle, and/or to the one or more biomolecules of interest}.

In certain embodiments, a concentration of the crosslinking agent is selected to avoid over-fixation of the vesicles [e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the concentration is selected based on a size of the vesicles; e.g., wherein the concentration is less than or approximately equal to 2% (e.g., about 1% or less; about 0.5% or less)].

In certain embodiments, the method of any one of the preceding claims, wherein step (b) comprising incubating the vesicles with the permeabilization agent for a duration selected to maintain integrity of a membrane of the vesicles (e.g., to avoid damaging and/or rupturing the membrane; e.g., wherein the duration is selected based on a size of the vesicles; e.g., wherein the duration is less than or approximately equal to 1 hour, e.g., about 30 minutes, e.g., about 10 minutes or less).

In certain embodiments, a concentration of the permeabilization agent in the permeabilization solution is selected to maintain integrity of a membrane of the vesicles (e.g., wherein the concentration of the permeabilization agent is selected based on a size of the vesicles; e.g., wherein a total concentration of detergent within the permeabilization solution is less than or approximately equal to 0.5%, e.g., about 0.2% or less, about 0.1% or less, about 0.05% or less).

In certain embodiments, the top surface of the substrate comprises one or more capture agents (e.g., antibodies), each capture agent specific to a particular target agent (e.g., antigen) of one or more target agents associated with (e.g., expressed on a surface of) at least a portion of the vesicles.

In certain embodiments, the particular target agent to which each of at least a portion of the one or more capture agents are specific is a surface marker (e.g., surface receptor, integrins, etc.) associated with a particular disease and/or condition (e.g., cancer).

In certain embodiments, (i) the one or more capture agents comprise an antibody specific to a cancer associated protein (e.g., an anti-CD63 antibody, an anti-CD81 antibody, an anti-CD9 antibody, an anti-CD171 antibody) and/or (ii) the one or more target agents comprise one or more cancer associated proteins (e.g., a CD63 protein, a CD81 protein, a CD9 protein, a CD171 protein).

In certain embodiments, the one or more biomolecules of interest comprise one or more proteins {e.g., one or more p-Tau protein species (e.g., two or more different p-Tau proteins); apoptotic linked-gene-product 2 (ALG-2) interacting protein X (ALIX); syntenin; syndecan; tumor susceptibility gene 101 (TSG101); one or more fluorescent proteins [e.g., fluorescent expression reporters, such as green fluorescent protein (GFP)]; e.g., one or more proteins from one or more of the following classes of proteins: enzymes; structural proteins; signaling proteins; regulatory proteins; transport proteins; sensory proteins; motor proteins; deference proteins; storage proteins; e.g., one or more peripheral proteins (e.g., outside cell membrane); e.g., one or more integral proteins}.

In certain embodiments, the one or more biomolecules of interest comprise one or more nucleic acids [e.g., nucleic acid fragments; e.g., a DNA oligonucleotide; e.g., an RNA oligonucleotide (e.g., a non-coding RNA); e.g., nucleic acid (e.g., DNA and/or RNA) in combination with protein; e.g., DNA and RNA combination oligonucleotides; e.g., locked nucleic acid (LNA) and/or other modified base].

In certain embodiments, step (c) comprises contacting the vesicles with one or more cargo labeling solutions, each comprising at least one of the one or more fluorescent cargo labels and a blocking agent (e.g., bovine serum albumin (BSA)) (e.g., wherein the cargo labeling solution comprises the one or more fluorescent cargo labels diluted in a blocking buffer).

In certain embodiments, a concentration of each of at least a portion of the fluorescent cargo labels (e.g., within the fluorescent cargo labeling solution) is about 1 microgram per milliliter or less.

In certain embodiments, the method comprises: [e.g., following step (a) (e.g., so as to label with the vesicles with the vesicle detection agent after capture); e.g., before step (a) (e.g., so as to label the vesicles with the vesicle detection agent prior to capture, e.g., in-solution)], contacting the vesicles with a fluorescent vesicle detection agent (e.g., antibody) specific to a particular target agent (e.g., antigen) (e.g., a target agent different from the one or more target agents to which the one or more capture agents are specific; e.g., a target agent that is the same as one of the one or more target agents to which the one or more capture agents are specific) associated with (e.g., expressed on a surface of) at least a portion of the vesicles, thereby labeling the vesicles (e.g., irrespective of their cargo) with the fluorescent vesicle detection agent; at step (d), exciting the fluorescent vesicle detection agent (e.g., in addition to the fluorescent cargo labels); and at step (e), detecting, with the one or more detectors, fluorescent light emitted from the fluorescent vesicle detection agent as a result of excitation by the excitation light (e.g., thereby obtaining a fluorescent image that can be used to detect presence of vesicles, e.g., and then compared with fluorescent images of the cargo labels to determine which/how many/what percentage of the vesicles comprise a particular biomolecule of interest).

In certain embodiments, step (e) comprises imaging the top surface of the substrate at one or more fluorescent wavelengths, each corresponding to an emission wavelength of (a fluorescent species of) a fluorescent cargo label, thereby obtaining one or more fluorescent images, each associated with a particular fluorescent cargo label and the particular biomolecule of interest to which the particular fluorescent cargo label is specific.

In certain embodiments, step (g) comprises: receiving and/or accessing, by a processor of a computing device, the one or more fluorescent images; identifying, by the processor, within each of at least a portion of the one or more fluorescent images, a plurality of discrete points of fluorescent emission, each determined to originate from within a vesicle [e.g., as opposed to background auto-fluorescence and/or signal from non-specifically bound fluorescent cargo label (e.g., on the top surface of the reflective substrate, but not within a vesicle), e.g., by identifying localized regions of high intensity within the fluorescent images, e.g., by performing a spatial cross-correlation with a point spread function]; and using the discrete points of fluorescent emission to detect and/or quantify, by the processor, the portion of the one or more particular biomolecules of interest [e.g., by summing, within each of the portion of the fluorescent images, the discrete points of fluorescent image; e.g., by identifying, within two or more of the fluorescent images, discrete points of fluorescent emission originating from same vesicles, e.g., to identify (e.g., presence, prevalence, rate) of co-expression of two or more biomolecules of interest].

In certain embodiments, the method comprises: (h) directing illumination light toward the top surface of the substrate, thereby illuminating the captured vesicles along with the substrate; and (i) detecting, with the one or more detectors, a label-free signal corresponding to a portion of the illumination light that is (A) scattered by the vesicles and/or (B) reflected by the substrate.

In certain embodiments, the imaging is performed using a high magnification objective lens having sufficiently high magnification and resolution to detect the fluorescent light emitted from the fluorescently labeled vesicles situated on the top surface of the substrate. In certain embodiments, wherein the high magnification objective lens has a magnification ranging from about 4× to about 100× (e.g., 4×, 10×, 20×, 40×, 60×, 100×). In certain embodiments, the high magnification objective lens has a numerical aperture ranging from about 0.1 and about 1.3 (e.g., 0.13, 0.3, 0.5. 0.75, 0.85, 1.25, 1.3).

In certain embodiments, step (g) comprises using the detected label-free signal along with the detected fluorescent light to detect and/or quantify the portion of the one or more biomolecules of interest.

In certain embodiments, step (i) comprises imaging the top surface of the substrate at one or more wavelengths of the illumination light, thereby obtaining one or more label-free images, each associated with a particular illumination wavelength. In certain embodiments, step (g) comprises: receiving and/or accessing, by a processor of a computing device, the one or more label-free images; identifying, by the processor, a plurality of vesicle locations on the top surface of the substrate using the one or more label-free images [e.g., by identifying localized regions of high scattering intensity within the label-free images, e.g., by performing a spatial cross-correlation with a point spread function]; and using the identified vesicle locations (e.g., in combination with the detected fluorescent light) to detect and/or quantify the portion of the one or more particular biomolecules of interest [e.g., by identifying vesicle locations from which fluorescent emission is detected, e.g., thereby determining a fraction, prevalence, etc. of expression of one or more particular biomolecules of interest].

In certain embodiments, the substrate is a reflective substrate comprising an optical interference coating comprising a stack of one or more layers (e.g., thin, semi-transparent layers)[e.g., such that the top surface of the reflective substrate corresponds to a top surface (e.g., a top surface of a top layer) of the optical interference coating], wherein a thickness and/or material of each of the one or more layers in the stack is such that: (A) excitation of and/or emission of fluorescent light from one or more of the fluorescent cargo labels is enhanced [e.g., relative to that which would be observed were the vesicles deposited on a bare substrate, without an optical interference coating (e.g., a Silicon substrate; e.g., a glass slide)], and/or (B) a label free signal, obtained by detection of light scattered by the vesicles in response to illumination by illumination light, is enhanced [e.g., relative to that which would be observed were the vesicles deposited on a bare substrate, without an optical interference coating (e.g., a Silicon substrate; e.g., a glass slide)]. In certain embodiments, the reflective substrate has a reflectance greater than 25% (e.g., greater than 30%, e.g., greater than 40%, e.g., greater than 50%, e.g., greater than 60%, greater than 70%, greater than 80% or more) at one or more particular wavelengths (e.g., a wavelength of the illumination light; e.g., an excitation and/or emission wavelength of at least a portion of the one or more fluorescent cargo labels).

In another aspect, the invention is directed to a method of isolating, permeabilizing, and labeling vesicles [e.g., nanovesicles; e.g., extracellular vesicles (e.g., exosomes); e.g., viruses; e.g., virus-like particles. e.g., liposomes] and their biomolecular cargo (e.g., one or more protein species potentially present within at least a portion of the vesicles), the method comprising: (a) contacting a surface of a substrate [e.g., a top surface of a substantially planer substrate (e.g., a reflective substrate); e.g., beads (e.g., magnetic beads)] with a sample comprising the vesicles [e.g., a sample obtained from a subject; e.g., a complex biological sample, such as blood, plasma, saliva, etc.; e.g., a processed sample (e.g., comprising vesicles in a buffer)], thereby capturing one or more vesicles present in the sample [e.g., wherein the surface of the substrate comprises one or more capture agents (e.g., antibodies), each capture agent specific to a particular target agent (e.g., antigen) associated with (e.g., expressed on a surface of) at least a portion of the vesicles]; (b) [e.g., following step (a) (e.g., so as to permeabilize the vesicles after capture); e.g., before step (a) (e.g., so as to permeabilize the vesicles prior to capture, e.g., in-solution)], contacting the vesicles with a permeabilization solution comprising a permeabilization agent [e.g., a detergent (e.g., Triton™)] thereby permeabilizing the captured vesicles, wherein a duration with which the vesicles are incubated with the permeabilization solution and/or a concentration of the permeabilization agent in the permeabilization solution is selected to maintain integrity of a membrane of the vesicles; and (c) following step (b), contacting the vesicles with one or more fluorescent cargo labels {e.g., a protein (e.g., antibody), a nucleic acid [e.g., an DNA oligonucleotide (e.g., a DNA aptamer), an RNA oligonucleotide (e.g., an RNA aptamer)], a peptide, a dye, etc.}, wherein each fluorescent cargo label is: (i) specific to a particular biomolecule of interest of one or more biomolecules of interest (e.g., and potentially present in at least a portion of the vesicles) and (ii) comprises a particular fluorescent species, thereby labeling the biomolecular cargo (e.g., the one or more biomolecules of interest) within the vesicles.

In certain embodiments, the method comprises performing step (b) and (c) following step (a), so as to permeabilize and label the vesicles after they are captured.

In certain embodiments, the method comprises performing step (b) and step (c) before step (a), so as to permeabilize and label the vesicles before they are captured.

In certain embodiments, rein the vesicles are less than or approximately equal to 1 micron in diameter (e.g., about 500 nm or less in diameter or less; about 200 nm or less in diameter; about 150 nm or less in diameter) (e.g., ranging from about 10 nm to about 150 nm in diameter; e.g., ranging from about 30 to about 100 nm in diameter).

In certain embodiments, the vesicles are extracellular vesicles. In certain embodiments, the extracellular vesicles are exosomes.

In certain embodiments, the method comprises [e.g., following step (a) and/or before step (b) (e.g., so as to fix (crosslink) the vesicles after capture); e.g., after step (a) and/or before step (b) (e.g., so as to fix (crosslink) the vesicles prior to capture (e.g., in-solution))] contacting the vesicles with a crosslinking agent [e.g., paraformaldehyde (PFA), glutaraldehyde, DENT's fixative (e.g., methanol with BSA), and the like], thereby fixing (crosslinking) the vesicles (e.g., wherein the crosslinking agent does not require a dehydration step)[e.g., other fixatives include, e.g., alcohols (e.g., methanol, ethanol), acetone, formaldehyde, formalin].

In certain embodiments, the method comprises incubating the vesicles with the crosslinking agent for a duration selected to avoid over-fixation of the vesicles {e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the duration is selected based on a size of the vesicles; e.g., wherein the duration is less than or approximately equal to 1 hour (e.g., about 30 minutes or less; about 10 minutes or less); e.g., and subsequently performing a wash step [e.g., with buffer (e.g., HEPES buffer, water, PBS)], e.g., to remove excess crosslinking agent and preserve the one or more target agents present on a surface of the vesicle, and/or to the one or more biomolecules of interest}.

In certain embodiments, a concentration of the crosslinking agent is selected to avoid over-fixation of the vesicles [e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the concentration is selected based on a size of the vesicles; e.g., wherein the concentration is less than or approximately equal to 2% (e.g., about 1% or less; about 0.5% or less)].

In certain embodiments, step (b) comprises incubating the vesicles with the permeabilization agent for a duration selected to maintain integrity of a membrane of the vesicles (e.g., to avoid damaging and/or rupturing the membrane; e.g., wherein the duration is selected based on a size of the vesicles; e.g., wherein the duration is less than or approximately equal to 1 hour, e.g., about 30 minutes, e.g., about 10 minutes or less).

In certain embodiments, a concentration of the permeabilization agent in the permeabilization solution is selected to maintain integrity of a membrane of the vesicles (e.g., wherein the concentration of the permeabilization agent is selected based on a size of the vesicles; e.g., wherein a total concentration of detergent within the permeabilization solution is less than or approximately equal to 0.5%, e.g., about 0.2% or less, about 0.1% or less, about 0.05% or less).

In certain embodiments, the surface of the substrate comprises one or more capture agents (e.g., antibodies), each capture agent specific to a particular target agent (e.g., antigen) of one or more target agents associated with (e.g., expressed on a surface of) at least a portion of the vesicles. In certain embodiments, the particular target agent to which each of at least a portion of the one or more capture agents are specific is a surface marker (e.g., surface receptor, integrins, etc.) associated with a particular disease and/or condition (e.g., cancer). In certain embodiments, (i) the one or more capture agents comprise an antibody specific to a cancer associated protein (e.g., an anti-CD63 antibody, an anti-CD81 antibody, an anti-CD9 antibody, an anti-CD171 antibody) and/or (ii) the one or more target agents comprise one or more cancer associated proteins (e.g., a CD63 protein, a CD81 protein, a CD9 protein, a CD171 protein).

In certain embodiments, the one or more biomolecules of interest comprise one or more proteins {e.g., one or more p-Tau protein species (e.g., two or more different p-Tau proteins); apoptotic linked-gene-product 2 (ALG-2) interacting protein X (ALIX); syntenin; syndecan; tumor susceptibility gene 101 (TSG101); one or more fluorescent proteins [e.g., fluorescent expression reporters, such as green fluorescent protein (GFP)]; e.g., one or more proteins from one or more of the following classes of proteins: enzymes; structural proteins; signaling proteins; regulatory proteins; transport proteins; sensory proteins; motor proteins; deference proteins; storage proteins; e.g., one or more peripheral proteins (e.g., outside cell membrane); e.g., one or more integral proteins}.

In certain embodiments, the one or more biomolecules of interest comprise one or more biomolecules of interest comprise one or more nucleic acids [e.g., nucleic acid fragments; e.g., a DNA oligonucleotide; e.g., an RNA oligonucleotide (e.g., a non-coding RNA); e.g., nucleic acid (e.g., DNA and/or RNA) in combination with protein; e.g., DNA and RNA combination oligos; e.g., locked nucleic acid (LNA) and/or other modified base].

In certain embodiments, step (c) comprises contacting the vesicles with one or more cargo labeling solutions, each comprising at least one of the one or more fluorescent cargo labels and a blocking agent (e.g., bovine serum albumin (BSA)) (e.g., wherein the cargo labeling solution comprises the one or more fluorescent cargo labels diluted in a blocking buffer).

In certain embodiments, a concentration of each of at least a portion of the fluorescent cargo labels (e.g., within the fluorescent cargo labeling solution) is about 1 microgram per milliliter or less.

In certain embodiments, the method comprises: [e.g., following step (a) (e.g., so as to label with the vesicles with the vesicle detection agent after capture); e.g., before step (a) (e.g., so as to label the vesicles with the vesicle detection agent prior to capture, e.g., in-solution)] contacting the vesicles with a fluorescent vesicle detection agent (e.g., antibody) specific to a particular target agent (e.g., antigen) (e.g., a target agent different from the one or more target agents to which the one or more capture agents are specific; e.g., a target agent that is the same as one of the one or more target agents to which the one or more capture agents are specific) associated with (e.g., expressed on a surface of) at least a portion of the vesicles, thereby labeling the vesicles (e.g., irrespective of their cargo) with the fluorescent vesicle detection agent.

In another aspect, the invention is directed to a kit for isolating, permeabilizing, and labeling vesicles [e.g., nanovesicles; e.g., extracellular vesicles (e.g., exosomes); e.g., viruses; e.g., virus-like particles; e.g., liposomes] and their biomolecular cargo (e.g., one or more protein species potentially present within at least a portion of the nanoparticles), the kit comprising: (a) a pre-mixed permeabilization solution comprising a permeabilization agent; and (b) one or more pre-mixed cargo labeling solutions, each comprising one or more fluorescent cargo labels [e.g., and a blocking agent (e.g., bovine serum albumin (BSA)) (e.g., wherein the cargo labeling solution comprises the one or more fluorescent cargo labels diluted in a blocking buffer)], wherein each fluorescent cargo label is (i) specific to a particular biomolecule of interest of one or more biomolecules of interest (e.g., and potentially present in at least a portion of the vesicles) and (ii) comprises a particular fluorescent species.

In certain embodiments, the kit further comprises one or more pre-mixed capture agent solutions, each comprising one or more capture agents (e.g., antibodies), wherein each capture agent is specific to a particular target agent (e.g., antigen) associated with (e.g., expressed on a surface of) at least a portion of the vesicles. In certain embodiments, the kit further comprises a pre-spotted substrate, wherein the pre-spotted substrate comprises one or more capture agent spots, each of the one or more capture agent spots comprising a particular capture agent (e.g., antibody) specific to a particular target agent (e.g., antigen) associated with (e.g., expressed on a surface of) at least a portion of the vesicles.

In certain embodiments, the particular target agent to which each of at least a portion of the one or more capture agents are specific is a surface marker (e.g., surface receptor, integrins, etc.) associated with a particular disease and/or condition (e.g., cancer).

In certain embodiments, (i) the one or more capture agents comprise an antibody specific to a cancer associated protein (e.g., an anti-CD63 antibody, an anti-CD81 antibody, an anti-CD9 antibody, an anti-CD171 antibody) and/or (ii) the one or more target agents comprise one or more cancer associated proteins (e.g., a CD63 protein, a CD81 protein, a CD9 protein, a CD171 protein).

In certain embodiments, the kit comprises a fixing solution comprising a crosslinking agent [e.g., paraformaldehyde (PFA), gluteraldehyde, DENT's fixative (e.g., methanol with BSA), and the like] for fixing (crosslinking) the vesicles (e.g., wherein the crosslinking agent does not require a dehydration step)[e.g., other fixatives include, e.g., alcohols (e.g., methanol, ethanol), acetone, formaldehyde, formalin]. In certain embodiments, a concentration of the crosslinking agent in the fixing solution is selected to avoid over-fixation of the vesicles [e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the concentration is selected based on a size of the vesicles; e.g., wherein the concentration is less than or approximately equal to 2% (e.g., about 1% or less; about 0.5% or less)].

In certain embodiments, a concentration of the permeabilization agent in the permeabilization solution is selected to maintain integrity of a membrane of the vesicles (e.g., wherein the concentration of the permeabilization agent is selected based on a size of the vesicles; e.g., wherein a total concentration of detergent within the permeabilization solution is less than or approximately equal to 0.5%, e.g., about 0.2% or less, about 0.1% or less, about 0.05% or less).

In certain embodiments, the one or more biomolecules of interest comprise one or more proteins {e.g., one or more p-Tau protein species (e.g., two or more different p-Tau proteins); apoptotic linked-gene-product 2 (ALG-2) interacting protein X (ALIX); syntenin; syndecan; tumor susceptibility gene 101 (TSG101); one or more fluorescent proteins [e.g., fluorescent expression reporters, such as green fluorescent protein (GFP)]}.

In certain embodiments, the one or more biomolecules of interest comprise one or more biomolecules of interest comprise one or more nucleic acids [e.g., nucleic acid fragments; e.g., a DNA oligonucleotide; e.g., an RNA oligonucleotide (e.g., a non-coding RNA); e.g., nucleic acid (e.g., DNA and/or RNA) in combination with protein; e.g., DNA and RNA combination oligos; e.g., locked nucleic acid (LNA) and/or other modified base].

In certain embodiments, a concentration of each of at least a portion of the fluorescent cargo labels (e.g., within the fluorescent cargo labeling solution) is about 1 microgram per milliliter or less.

In certain embodiments, the kit comprises a vesicle detection solution comprising a fluorescent vesicle detection agent (e.g., antibody) specific to a particular target agent (e.g., antigen) (e.g., a target agent different from the one or more target agents to which the one or more capture agents are specific; e.g., a target agent that is the same as one of the one or more target agents to which the one or more capture agents are specific) associated with (e.g., expressed on a surface of) at least a portion of the vesicles (e.g., labeling the vesicles (e.g., irrespective of their cargo) with the fluorescent vesicle detection agent);

In certain embodiments, the kit comprises a reflective substrate comprising an optical interference coating comprising a stack of one or more layers (e.g., thin, semi-transparent layers)[e.g., such that the top surface of the reflective substrate corresponds to a top surface (e.g., a top surface of a top layer) of the optical interference coating], wherein a thickness and/or material of each of the one or more layers in the stack is such that: (A) excitation of and/or emission of fluorescent light from one or more of the fluorescent cargo labels is enhanced [e.g., relative to that which would be observed were the vesicles deposited on a bare substrate, without an optical interference coating (e.g., a Silicon substrate; e.g., a glass slide)], and/or (B) a label free signal, obtained by detection of light scattered by the vesicles in response to illumination by illumination light, is enhanced [e.g., relative to that which would be observed were the vesicles deposited on a bare substrate, without an optical interference coating (e.g., a Silicon substrate; e.g., a glass slide)]. In certain embodiments, the reflective substrate has a reflectance greater than 25%, (e.g., greater than 30%, e.g., greater than 40%, e.g., greater than 50%, e.g., greater than 60%, greater than 70%, greater than 80% or more) at one or more particular wavelengths (e.g., a wavelength of the illumination light; e.g., an excitation and/or emission wavelength of a at least a portion of the one or more fluorescent cargo labels).

In another aspect, the invention is directed to a system for isolating, labeling, and imaging vesicles [e.g., nanovesicles; e.g., extracellular vesicles (e.g., exosomes); e.g., viruses; e.g., virus-like particles; e.g., liposomes] and their biomolecular cargo (e.g., one or more protein species potentially present within at least a portion of the nanoparticles), the system comprising: (a) a kit for isolating, permeabilizing, and labeling vesicles [e.g., nanovesicles; e.g., extracellular vesicles (e.g., exosomes); e.g., viruses; e.g., virus-like particles; e.g., liposomes] and their biomolecular cargo (e.g., one or more protein species potentially present within at least a portion of the nanoparticles); (b) a mount for holding a substrate (e.g., wherein the mount is a standard microscope mount); (c) one or more excitation light sources aligned with respect to the mount so as to and direct excitation light toward a top surface of the substrate (e.g., when held by the mount), so as to provide for excitation of one or more fluorescently labeled (e.g., fluorescent cargo labels) vesicles situated on the top surface of the substrate; (d) one or more detectors aligned with respect to the mount and operable to detect fluorescent light emitted from the fluorescently labeled vesicles situated on the top surface of the substrate; (e) a processor of a computing device; and (f) a memory having instructions stored thereon, wherein the instructions, when executed by the processor, cause the processor to: receive and/or access data corresponding to the detected fluorescent light; and use the data corresponding to the detected fluorescent light to detect and/or quantify the biomolecular cargo of the vesicles.

In certain embodiments, the kit comprises: (A) a pre-mixed permeabilization solution comprising a permeabilization agent; and (B) one or more pre-mixed cargo labeling solutions, each comprising one or more fluorescent cargo labels [e.g., and a blocking agent (e.g., bovine serum albumin (BSA)) (e.g., wherein the cargo labeling solution comprises the one or more fluorescent cargo labels diluted in a blocking buffer)], wherein each fluorescent cargo label is (i) specific to a particular biomolecule of interest of one or more biomolecules of interest (e.g., and potentially present in at least a portion of the vesicles) and (ii) comprises a particular fluorescent species.

In certain embodiments, the one or more detectors are each aligned with respect a high magnification objective lens having sufficiently high magnification and resolution to detect the fluorescent light emitted from the fluorescently labeled vesicles situated on the top surface of the substrate. In certain embodiments, the high magnification objective lens has a magnification ranging from about 4× to about 100× (e.g., 4×, 10×, 20×, 40×, 60×, 100×). In certain embodiments, the high magnification objective lens has a numerical aperture ranging from about 0.1 and about 1.3 (e.g., 0.13, 0.3, 0.5. 0.75, 0.85, 1.25, 1.3).

In certain embodiments, the kit further comprises one or more pre-mixed capture agent solutions, each comprising one or more capture agents (e.g., antibodies), wherein each capture agent is specific to a particular target agent (e.g., antigen) associated with (e.g., expressed on a surface of) at least a portion of the vesicles.

In certain embodiments, the kit further comprises a pre-spotted substrate, wherein the pre-spotted substrate comprises one or more capture agent spots, each of the one or more capture agent spot comprising a particular capture agent (e.g., antibody) specific to a particular target agent (e.g., antigen) associated with (e.g., expressed on a surface of) at least a portion of the vesicles.

In certain embodiments, the particular target agent to which each of at least a portion of the one or more capture agents are specific is a surface marker (e.g., surface receptor, integrins, etc.) associated with a particular disease and/or condition (e.g., cancer).

In certain embodiments, (i) the one or more capture agents comprise an antibody specific to a cancer associated protein (e.g., an anti-CD63 antibody, an anti-CD81 antibody, an anti-CD9 antibody, an anti-CD171 antibody) and/or (ii) the one or more target agents comprise one or more cancer associated proteins (e.g., a CD63 protein, a CD81 protein, a CD9 protein, a CD171 protein).

In certain embodiments, the kit comprises a fixing solution comprising a crosslinking agent [e.g., paraformaldehyde (PFA), gluteraldehyde, DENT's fixative (e.g., methanol with BSA), and the like] for fixing (crosslinking) the vesicles (e.g., wherein the crosslinking agent does not require a dehydration step)[e.g., other fixatives include, e.g., alcohols (e.g., methanol, ethanol), acetone, formaldehyde, formalin].

In certain embodiments, a concentration of the crosslinking agent in the fixing solution is selected to avoid over-fixation of the vesicles [e.g., to avoid damaging (e.g., rendering inactive and/or compromising a structure of; e.g., denaturing) one or more target agents present on a surface of the vesicle and/or to avoid damaging (e.g., rendering inactive and/or compromising a structure of; e.g., denaturing) the one or more biomolecules of interest; e.g., wherein the concentration is selected based on a size of the vesicles; e.g., wherein the concentration is less than or approximately equal to 2% (e.g., about 1% or less; about 0.5% or less)].

In certain embodiments, a concentration of the permeabilization agent in the permeabilization solution is selected to maintain integrity of a membrane of the vesicles (e.g., wherein the concentration of the permeabilization agent is selected based on a size of the vesicles; e.g., wherein a total concentration of detergent within the permeabilization solution is less than or approximately equal to 0.5%, e.g., about 0.2% or less, about 0.1% or less, about 0.05% or less).

In certain embodiments, the one or more biomolecules of interest comprise one or more proteins {e.g., one or more p-Tau protein species (e.g., two or more different p-Tau proteins); apoptotic linked-gene-product 2 (ALG-2) interacting protein X (ALIX); syntenin; syndecan; tumor susceptibility gene 101 (TSG101); one or more fluorescent proteins [e.g., fluorescent expression reporters, such as green fluorescent protein (GFP)]}.

In certain embodiments, the one or more biomolecules of interest comprise one or more biomolecules of interest comprise one or more nucleic acids [e.g., nucleic acid fragments; e.g., a DNA oligonucleotide; e.g., an RNA oligonucleotide (e.g., a non-coding RNA); e.g., nucleic acid (e.g., DNA and/or RNA) in combination with protein; e.g., DNA and RNA combination oligos; e.g., locked nucleic acid (LNA) and/or other modified base].

In certain embodiments, a concentration of each of at least a portion of the fluorescent cargo labels (e.g., within the fluorescent cargo labeling solution) is about 1 microgram per milliliter or less.

In certain embodiments, the kit comprises a vesicle detection solution comprising a fluorescent vesicle detection agent (e.g., antibody) specific to a particular target agent (e.g., antigen) (e.g., a target agent different from the one or more target agents to which the one or more capture agents are specific; e.g., a target agent that is the same as one of the one or more target agents to which the one or more capture agents are specific) associated with (e.g., expressed on a surface of) at least a portion of the vesicles (e.g., labeling the vesicles (e.g., irrespective of their cargo) with the fluorescent vesicle detection agent).

In certain embodiments, the system comprises a reflective substrate comprising an optical interference coating comprising a stack of one or more layers (e.g., thin, semi-transparent layers)[e.g., such that the top surface of the reflective substrate corresponds to a top surface (e.g., a top surface of a top layer) of the optical interference coating], wherein a thickness and/or material of each of the one or more layers in the stack is such that: (A) excitation of and/or emission of fluorescent light from one or more of the fluorescent cargo labels is enhanced [e.g., relative to that which would be observed were the vesicles deposited on a bare substrate, without an optical interference coating (e.g., a Silicon substrate; e.g., a glass slide)], and/or (B) a label free signal, obtained by detection of light scattered by the vesicles in response to illumination by illumination light, is enhanced [e.g., relative to that which would be observed were the vesicles deposited on a bare substrate, without an optical interference coating (e.g., a Silicon substrate; e.g., a glass slide)]. In certain embodiments, the reflective substrate has a reflectance greater than 25%, (e.g., greater than 30%, e.g., greater than 40%, e.g., greater than 50%, e.g., greater than 60%, greater than 70%, greater than 80% or more) at one or more particular wavelengths (e.g., a wavelength of the illumination light; e.g., an excitation and/or emission wavelength of a at least a portion of the one or more fluorescent cargo labels).

Elements of embodiments involving one aspect of the invention (e.g., methods) can be applied in embodiments involving other aspects of the invention, and vice versa.

### BRIEF DESCRIPTION OF THE FIGURES

The foregoing and other objects, aspects, features, and advantages of the present disclosure will become more apparent and better understood by referring to the following description taken in conduction with the accompanying drawings, in which:

FIG. 1A is a block flow diagram showing a process for isolating, labeling, and imaging extracellular vesicles and their biomolecular cargo, according to an illustrative embodiment of the present disclosure.

FIG. 1B is a block flow diagram showing a process for isolating, permeabilizing, and labeling extracellular vesicles and their biomolecule cargo, according to an illustrative embodiment of the present disclosure.

FIG. 2 is a diagram of an exemplary system for use in detection of particles bound to a substrate, according to an illustrative embodiment of the present disclosure.

FIG. 3 is a diagram of light reflected off of a substrate and scattered by a particle bound to the substrate, according to an illustrative embodiment of the present disclosure.

FIG. 4A is an illustration showing an instrument for imaging optical substrates as described herein, e.g., for detection of particles (e.g., exosomes), e.g., for the co-localization of exosomes with biomolecular cargo of interest, according to an illustrative embodiment.

FIG. 4B is an illustration of a reflective chip (substrate), as described herein, according to an illustrative embodiment.

FIG. 4C is an illustration of the reflective chip disposed within a microfluidic cassette, which allows flowing of a sample over the substrate, according to an illustrative embodiment.

FIG. 4D is an illustration of an array of capture agents (e.g., antibodies) on the substrate, as described herein, according to an illustrative embodiment.

FIG. 5 is a block flow diagram of a process for locating and/or classifying particles via simultaneous detection of fluorescence and single-particle scattering, according to an illustrative embodiment.

FIG. 6 is a block flow diagram of a process for locating and/or classifying particles via detection of enhanced fluorescence from a plurality of distinct fluorescent species within and/or on a surface of the particles.

FIG. 7 is a set of fluorescent images demonstrating the effect of fluorescent labeling on samples were incubated in the presence (right) or absence (left) of PFA.

FIG. 8 is a set of images showing fluorescent markers bound to permeabilized vesicles, as prepared with an exemplary method described herein.

FIG. 9 is a set of images showing fluorescent markers bound to permeabilized vesicles, as prepared with an exemplary method described herein.

FIG. 10A is a set of fluorescence images of non-permeabilized extracellular vesicles (EVs) bound to an optical substrate (or chip) using an anti-CD63 antibody and labeled with a fluorescent marker, as described herein.

FIG. 10B is a graph showing a number of fluorescent particles identified in each of the fluorescence channels for identifying CD63 capture and a negative control.

FIG. 11A is a set of fluorescence images of extracellular vesicles (EVs) bound to an optical substrate (or chip) using an anti-CD63 antibody after 15 minutes of permeabilization.

FIG. 11B is a graph depicting a number of fluorescent particles identified in each of the fluorescence channels for identifying CD63 capture and a negative control.

FIG. 12 is a graph showing measurements of captured extracellular vesicles (EVs) from human plasma obtained via a label-free method of SP-IRIS imaging, according to an embodiment of the present disclosure.

FIG. 13 is a set of fluorescent images depicting the effect of antibody incubation time on fluorescence.

FIG. 14 is a set of images showing effects of Triton™-X concentration on permeabilization.

FIG. 15 is a set of images showing the effect of permeabilization time on labeling.

FIG. 16 is a block diagram of an exemplary cloud computing environment, used in certain embodiments.

FIG. 17 is a block diagram of an example computing device and an example mobile computing device used in certain embodiments.

FIG. 18 is a graph showing detection of fluorescently tagged Syntenin in raw and purified human plasma.

FIG. 19 is a set of fluorescence images of extracellular vesicles (EVs) bound to an optical substrate (or chip) using an anti-CD9 antibody (top) or a negative control (bottom) and labeled with a fluorescent marker, as described herein.

FIG. 20A is a graph showing the detection of fluorescently tagged Syntenin and CD9 in extracellular vesicles (EVs) obtained from mouse cerebrospinal fluid (CSF).

FIG. 20B is a graph showing the detection of fluorescently tagged Syntenin and CD9 in cell-line derived extracellular vesicles (EVs) obtained from mouse brain endothelial cells (bEnd3).

FIG. 20C is a graph showing the detection of fluorescently tagged Syntenin and CD9 in cell-line derived extracellular vesicles (EVs) obtained from mouse fibroblast cells (NIH/3T3).

FIG. 21A is a fluorescent image of extracellular vesicles (EVs) obtained from cerebrospinal fluid (CSF) and immunolabeled with anti-mCD9 and anti-mSyntenin.

FIG. 21B is a fluorescent image of extracellular vesicles (EVs) obtained from mouse brain endothelial cells (bEnd3) and immunolabeled with anti-mCD9 and anti-mSyntenin.

FIG. 21C is a fluorescent image of extracellular vesicles (EVs) obtained from mouse fibroblast cells (NIH3T3) and immunolabeled with anti-mCD9 and anti-mSyntenin.

FIG. 22 is a set of graphs depicting the diameter EVs captured on a substrate as compared with the fluorescent intensity of their respective immunolabels.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

Antibody: As used herein, the term “antibody” or “antibody molecule” refers to immunoglobulin molecules and immunologically active portions of immunoglobulin (Ig) molecules, i.e., molecules that contain an antigen binding site that specifically binds, e.g., immunoreacts with, an antigen. By “specifically binds” or “immunoreacts with” is meant that the antibody reacts with one or more antigenic determinants of the desired antigen and has a lower affinity for other polypeptides, e.g., does not react with other polypeptides.

In embodiments, an antibody or antibody molecule encompasses full-length antibodies and antibody fragments. For example, a full-length antibody is an immunoglobulin (Ig) molecule (e.g., an IgG antibody) that is naturally occurring or formed by normal immunoglobulin gene fragment recombinatorial processes). In embodiments, an antibody or antibody molecule refers to an immunologically active, antigen-binding portion of an immunoglobulin molecule, such as an antibody fragment.

“Antigen Binding Site”, “Binding Portion”: The term “antigen-binding site,” or “binding portion” refers to the part of the immunoglobulin (Ig) molecule that participates in antigen binding. The antigen binding site is formed by amino acid residues of the N-terminal variable (“V”) regions of the heavy (“H”) and light (“L”) chains. Three highly divergent stretches within the variable regions of the heavy and light chains, referred to as hypervariable regions, are interposed between more conserved flanking stretches known as “framework regions,” or “FRs”. The term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The extent of the framework region and CDRs have been defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the amino acid order: FR1, CDR1, FR2, CDR2, FR3, CDR3, FR4.

Binding Agent, Binding Reagent, Capture Agent: As described herein, the term “binding reagent”, “capture agent” and “binding agent” are used interchangeably herein to refer to any entity that binds to a target of interest as described herein. In many embodiments, a capture agent of interest is one that binds specifically with its target in that it discriminates its target from other potential binding partners in a particular interaction contact. In general, a capture agent may be or comprise an entity of any chemical class (e.g., polymer, non-polymer, small molecule, polypeptide, carbohydrate, lipid, nucleic acid, etc.). In some embodiments, a capture agent is a single chemical entity. In some embodiments, a capture agent is a complex of two or more discrete chemical entities associated with one another under relevant conditions by non-covalent interactions. For example, those skilled in the art will appreciate that in some embodiments, a capture agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple capture agents through linkage of different specific binding moieties with the same generic binding moiety partner. In some embodiments, capture agents are or comprise peptides and/or polypeptides (including, e.g., antibodies or antibody fragments). In certain embodiments, the peptides and/or polypeptides may be further labeled with an isotope. In some embodiments, capture agents are or comprise antibodies (e.g., including monoclonal antibodies, polyclonal antibodies, bispecific antibodies, or antigen-binding fragments thereof, and antibody fragment including, ScFv, F(ab), F(ab′)2, Fv). In some embodiments, capture agents are or comprise small molecules. In some embodiments, capture agents are or comprise nucleic acids. In some embodiments, capture agents are aptamers. In some embodiments, capture agents are polymers. In some embodiments, capture agents are non-polymeric in that they lack polymeric moieties. In some embodiments, binding agents are or comprise carbohydrates. In certain embodiments, capture agents are or comprise nucleic acids, such as DNA or RNA. In certain embodiments as described herein, a capture agent may be present on the top surface of a substrate as described herein (e.g., an optical substrate, e.g., a reflective substrate). In certain preferable embodiments, a capture agent may be an antibody specific to a cancer associated protein (e.g., specific to a member of the tetraspanin family e.g., an anti-CD 63 antibody, an anti-CD 81 antibody, an anti-CD 9 antibody, an anti-CD171 antibody).

Biomarker: The term “biomarker” is used herein, consistent with its use in the art, to refer to a to an entity, event, or characteristic whose presence, level, degree, and/or type, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid (e.g., DNA, RNA), a polypeptide, a lipid, a carbohydrate, a small molecule, an inorganic agent (e.g., a metal or ion), or a combination thereof. In some embodiments, a biomarker is on a surface of a cell (e.g., a surface cell marker). In some embodiments, a biomarker is on a surface of a vesicle (e.g., an exosome). In some embodiments, a biomarker is internal to a vesicle. In some embodiments, a biomarker is a member of the tetraspanin family (e.g., CD9, CD63, CD81). In some embodiments, a biomarker is a member of the endosomal sorting complexes required for transport (e.g., ESCRT; TSG101, Alix). In some embodiments, a biomarker is a heat shock protein (e.g., Hsp60, Hsp70, Hsp90). In some embodiments, a biomarker is an extracellular vesicle (e.g., an extracellular vesicle having one or more surface markers of interest).

Cancer, tumor, tumor tissue: As used herein, the terms “cancer,” “tumor” or “tumor tissue” refer to an abnormal mass of tissue that results from excessive cell division, in certain cases tissue comprising cells which express, over-express, or abnormally express a hyperproliferative cell protein. A cancer, tumor or tumor tissue comprises “tumor cells” which are neoplastic cells with abnormal growth properties and no useful bodily function. Cancers, tumors, tumor tissue and tumor cells may be benign or malignant. A cancer, tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

Examples of cancer include, but are not limited to, carcinoma, lymphoma, blastoma, sarcoma, and leukemia or lymphoid malignancies. More particular examples of such cancers are noted below and include: squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

In some embodiments, the cancer is an adenocarcinoma. In some embodiments, the cancer is selected from breast, lung, head or neck, prostate, esophageal, tracheal, brain, liver, bladder, stomach, pancreatic, ovarian, uterine, cervical, testicular, colon, rectal, and skin. In some embodiments the caner is an adenocarcinoma of the breast, lung, head or neck, prostate, esophagus, trachea, brain, liver, bladder, stomach, pancreas, ovary, uterus cervix, testicular, colon, rectum, or skin. In some embodiments the cancer is selected from pancreatic, lung (e.g., small cell or non-small cell), and breast.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In certain embodiments cancers are detected using surface markers, surface receptors, and/or integrins.

Comprising: As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the present disclosure. Other than in the operating examples, or where otherwise indicated, all numbers expressing quantities of ingredients or reaction conditions used herein should be understood as modified in all instances by the term “about.”

Epitope: As used herein, the term “epitope” includes any protein determinant capable of specifically binding to an immunoglobulin, antibody fragment, e.g., an antibody fragment described herein, or a B cell receptor (BCR) (e.g., BCR comprising an immunoglobulin). Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Immunological binding, immunological binding properties, specifically binds, selectively binds: As used herein, the terms “immunological binding,” “immunological binding properties,” “specifically binds,” or “selectively binds” refer to non-covalent interactions of the type that occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kon) and the “off rate constant” (koff) can be determined by calculation of the concentrations and the actual rates of association and dissociation. See, e.g., Nature 361:186-87 (1993). The ratio of koff/kon facilitates the cancellation of all parameters not related to affinity, and is equal to the dissociation constant Kd. (See, generally, Davies et al. (1990) Annual Rev Biochem 59:439-473).

In some embodiments, an antibody molecule described herein specifically binds an antigen/epitope (e.g., autoantigen, e.g., islet autoantigen, e.g., insulin; or a B cell, e.g., autoantigen-specific B cell, insulin-specific B cell; or an autoantigen::BCR complex, e.g., insulin::BCR complex) when the equilibrium binding constant (Kd) is less than or equal to 1 μM, e.g., less than or equal to 100 nM, less than or equal to 10 nM, less than or equal to 100 pM, or less than or equal to about 1 pM, e.g., as measured by assays such as radioligand binding assays, ELISAs, surface plasmon resonance, equilibrium binding assays, or similar assays known to those skilled in the art.

Light Emitting Diode (LED): As used herein, a “light emitting diode (LED)” is an electronic light source based on the semiconductor diode. When the diode is forward biased (switched on), electrons are able to recombine with holes and energy is released in the form of light. This effect is called electroluminescence and the color of the light is determined by the energy gap of the semiconductor. The LED is usually small in area (less than 1 mm) with integrated optical components to shape its radiation pattern and assist in measures the fraction of light that passes through a given solution. In a spectrophotometer, a light from the lamp is guided through a monochromator, which picks light of one particular wavelength out of the continuous spectrum. This light passes through the sample that is being measured. After the sample, the intensity of the remaining light is measured with a photodiode or other light sensor, and the transmittance for this wavelength is then calculated. In short, the sequence of events in a spectrophotometer is as follows: the light source shines through the sample, the sample absorbs light, the detector detects how much light the sample has absorbed, the detector then converts how much light the sample absorbed into a number, the numbers are e are transmitted to a comparison module to be further manipulated (e.g., curve smoothing, baseline correction). Many spectrophotometers must be calibrated by a procedure known as “zeroing.” The absorbency of some standard substance is set as a baseline value, so the absorbencies of all other substances are recorded relative to the initial “zeroed” substance. The spectrophotometer then displays % absorbency (the amount of light absorbed relative to the initial reflection. Like a normal diode, the LED consists of a chip of semiconducting material impregnated, or doped, with impurities to create a p-n junction. As in other diodes, current flows easily from the p-side, or anode, to the n-side, or cathode, but not in the reverse direction. Charge-carriers—electrons and holes—flow into the junction from electrodes with different voltages. When an electron meets a hole, it falls into a lower energy level, and releases energy in the form of a photon. The wavelength of the light emitted, and therefore its color, depends on the band gap energy of the materials forming the p-n junction. In silicon or germanium diodes, the electrons and holes recombine by a non-radiative transition which produces no optical emission, because these are indirect band gap materials. The materials used for the LED have a direct band gap with energies corresponding to near-infrared, visible or near-ultraviolet light. LEDs are usually built on an n-type substrate, with an electrode attached to the p-type layer deposited on its surface. P-type substrates, while less common, occur as well. Many commercial LEDs, especially GaN/InGaN, also use sapphire substrate. Most materials used for LED production have very high refractive indices. This means that much light will be reflected back in to the material at the material/air surface interface. LEDs of use for the described technology, include but are not limited to:

| | Wavelength | Voltage | |
|---|---|---|---|
| Color | [nm] | [V] | Semiconductor Material |
| Infrared | λ > 760 | ΔV < 1.9 | Gallium arsenide (GaAs) |
| | | | Aluminum gallium arsenide (AlGaAs) |
| Red | 610 < λ < 760 | 1.63 < ΔV < 2.03 | Aluminum gallium arsenide (AlGaAs) |
| | | | Gallium arsenide phosphide (GaAsP) |
| | | | Aluminum gallium indium phosphide (AlGaInP) |
| | | | Gallium(III) phosphide (GaP) |
| Orange | 590 < λ < 610 | 2.03 < ΔV < 2.10 | Gallium arsenide phosphide (GaAsP) |
| | | | Aluminum gallium indium phosphide (AlGaInP) |
| | | | Gallium(III) phosphide (GaP) |
| Yellow | 570 < λ < 590 | 2.10 < ΔV < 2.18 | Gallium arsenide phosphide (GaAsP) |
| | | | Aluminum gallium indium phosphide (AlGaInP) |
| | | | Gallium(III) phosphide (GaP) |
| Green | 500 < λ < 570 | 2.18 < ΔV < 4.0 | Indium gallium nitride (InGaN)/Gallium(III) nitride (GaN) |
| | | | Gallium(III) phosphide (GaP) |
| | | | Aluminum gallium indium phosphide (AlGaInP) |
| | | | Aluminum gallium phosphide (AlGaP) |
| Blue | 450 < λ < 500 | 2.48 < ΔV < 3.7 | Zinc selenide (ZnSe) |
| | | | Indium gallium nitride (InGaN) |
| | | | Silicon carbide (SiC) as substrate |
| | | | Silicon (Si) as substrate - (under development) |
| Violet | 400 < λ < 450 | 2.76 < ΔV < 4.0 | Indium gallium nitride (InGaN) |
| Purple | multiple | 2.48 < ΔV < 3.7 | Dual blue/red LEDs, |
| | types | | blue with red phosphor, |
| | | | or white with purple plastic |
| Ultraviolet | λ < 400 | 3.1 < ΔV < 4.4 | diamond (C) |
| | | | Aluminum nitride (AlN) |
| | | | Aluminum gallium nitride (AlGaN) |
| | | | Aluminum gallium indium nitride (AlGaInN) - |
| | | | (down to 210 nm) |
| White | Broad | ΔV = 3.5 | Blue/UV diode with yellow phosphor |
| | spectrum | | |

Particle: A “particle,” as defined herein, refers to any target to be detected by the devices and methods described herein that has a radius from a few nanometers up to a few microns. In certain embodiments, a particle may be a vesicle (e.g., a nanovesicle, e.g., an extracellular vesicle, e.g., a liposome, e.g., an exosome). In certain embodiments, a particle may be an extracellular vesicle (e.g., a tumor derived circulating extracellular vesicle). In certain particular embodiments, a particle may be an exosome.

Monoclonal Antibody, mAb: The term “monoclonal antibody” or “mAb,” as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

Multispecific Antibody: A “multispecific antibody” is an antibody that can bind simultaneously to at least two targets that are of different structure, e.g., two different antigens, two different epitopes on the same antigen, or a hapten and/or an antigen or epitope. For example, one specificity can be for a B cell, e.g., an insulin-specific BCR on an insulin-specific B cell, and another specificity can be to a different antigen on a B cell. In another example, another specificity can be to a receptor on a phagocytosing cell, e.g., macrophage. In another example, another specificity can be to a receptor on a dendritic cell. Multispecific, multivalent antibodies are constructs that have more than one binding site, and the binding sites are of different specificity.

Polyclonal Antibody: The term “polyclonal antibody” refers to a mixture of different antibody molecules which react with more than one immunogenic determinant of an antigen. In embodiments, polyclonal antibodies can be isolated or purified from mammalian blood, secretions, or other fluids, or from eggs. In other embodiments, polyclonal antibodies are made up of a mixture of different monoclonal antibodies. In other embodiments, a polyclonal antibody can be produced as a recombinant polyclonal antibody.

Reflective Substrate: As used herein, reflective substrate is used to refer to a substrate for reflecting light back to a detector. As used herein, the term “reflective substrate” is intended to encompass a variety of substrates and/or substrate materials having various reflectance. In certain embodiments, the reflective substrate comprises a single layer. In certain embodiments, a reflective substrate comprises an oxide layer on a silicon base. In certain embodiments, the reflective substrate comprises multiple layers (e.g., as described in further detail herein).

In certain embodiments, a reflective substrate has a reflectance greater than a particular minimum value at one or more wavelengths and/or spectral ranges of interest. Exemplary spectral ranges include, but are not limited to, the UV spectral range, ranging from about 400 nm to 450 nm, the blue spectral range, ranging from about 460 nm to about 500 nm, the green spectral range, ranging from about 520 nm to about 560 nm, the red spectral range, ranging from about 640 nm to about 680 nm, and the deep red spectral range, ranging from about 710 nm to about 750 nm. For example, a reflective substrate may have a “reflectance” or “reflectivity” greater than or approximately equal to 25% (e.g., greater than 30%, e.g., greater than 40%, e.g., greater than 50%, e.g., greater than 60%, greater than 70%) across one or more wavelengths and/or spectral bands of interest. In certain embodiments, the reflective substrate has reflectance greater than 80% or more across one or more wavelengths and/or spectral bands of interest.

A reflective substrate may have a reflectance that varies according to a particular functional form, such as a sinuosoid, e.g., produced by optical interference effects, such that it has a particular reflectance at one or more wavelengths and/or spectral ranges of interest, but a relatively low reflectance at other wavelengths.

Sample, Biological Sample: As used herein the terms “sample” and “biological sample” means any sample, including, but not limited to cells, organisms, lysed cells, cellular extracts, nuclear extracts, components of cells or organisms, extracellular fluid, media in which cells are cultured, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears and prostatic fluid. In addition, a sample can be a viral or bacterial sample, a sample obtained from an environmental source, such as a body of polluted water (e.g., a lake), an air sample, or a soil sample, as well as a food industry sample (e.g., a food source believed to be contaminated).

Spectrograph, spectrophotometer: A “spectrograph” or “spectrometer”, as defined herein, is an optical instrument used to measure properties of light over a specific portion of the electromagnetic spectrum, typically used in spectroscopic analysis to identify materials. The variable measured is most often the intensity of light but may also, for instance, be the polarization state of the light. The independent variable is usually the wavelength of the light, normally expressed as a fraction of a meter, but sometimes expressed as a unit directly proportional to the photon energy, such as wavenumber or electron volts, which has a reciprocal relationship to wavelength. A spectrometer is used in spectroscopy for producing spectral lines and measuring their wavelengths and intensities. Spectrometer is a term that is applied to instruments that operate over a very wide range of wavelengths, from gamma rays and X-rays into the far infrared. If the region of interest is restricted to near the visible spectrum, the study is called spectrophotometry.

Spectrophotometry involves the use of a spectrophotometer. As defined herein, a “spectrophotometer” is a photometer (a device for measuring light intensity) that can measure intensity as a function of the color, or more specifically, the wavelength of light. There are many kinds of spectrophotometers. Among the most important distinctions used to classify them are the wavelengths they work with, the measurement techniques they use, how they acquire a spectrum, and the sources of intensity variation they are designed to measure. Other important features of spectrophotometers include the spectral bandwidth and linear range. There are two major classes of spectrophotometers; single beam and double beam. A double beam spectrophotometer measures the ratio of the light intensity on two different light paths, and a single beam spectrophotometer measures the absolute light intensity. Although ratio measurements are easier, and generally more stable, single beam instruments have advantages; for instance, they can have a larger dynamic range, and they can be more compact. Historically, spectrophotometers use a monochromator to analyze the spectrum, but there are also spectrophotometers that use arrays of photosensors. Especially for infrared spectrophotometers, there are spectrophotometers that use a Fourier transform technique to acquire the spectral information quicker in a technique called Fourier Transform InfraRed (FTIR) spectroscopy. The spectrophotometer quantitatively substance). The most common application of spectrophotometers is the measurement of light absorption, but they can be designed to measure diffuse or specular reflectance. Strictly, even the emission half of a luminescence instrument is a kind of spectrophotometer.

“Tag”, “label”: The terms “label” or “tag”, as used herein, refer to a composition capable of producing a detectable signal indicative of the presence of the target in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

“Vesicle”, “microvesicle”, “exosome”: The terms “microvesicle”, “vesicle”, and “exosome,” as used herein, refer to a membranous particle, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell. As used herein, the term “nanovesicles” refers to subcellular, substantially spherical bodies or membranous bodies such as liposomes, micelles, extracellular vesicles, exosomes, viruses, virus like particles, microbubbles, or unilamellar vesicles.

### DETAILED DESCRIPTION

It is contemplated that systems, architectures, devices, methods, and processes of the present disclosure encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present disclosure that consist essentially of, or consist of, the recited components, and that there are processes and methods according to the present disclosure that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Where there is any discrepancy in the meaning of a particular term, the meaning provided in the Definition section above is controlling.

Headers are provided for the convenience of the reader—the presence and/or placement of a header is not intended to limit the scope of the subject matter described herein.

It is understood that the detailed description and the following examples are illustrative only and are not to be taken as limitations upon the scope of the described technology. Various changes and modifications to the disclosed embodiments, which will be apparent to those, skilled in the art, may be made without departing from the spirit and scope of the described technology. Further, all patents, patent applications, and publications identified are expressly incorporated herein by reference for the purpose of describing and disclosing, for example, the methodologies described in such publications that might be used in connection with the described technology. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

Presented herein are systems and methods for detecting molecular cargo [e.g., protein, e.g., DNA, e.g., RNA (e.g., microRNA, e.g., non-coding RNA), e.g., dyes, e.g., aptamers] inside of particles (e.g., exosomes, e.g., liposomes, e.g., viruses, e.g., extracellular vesicles) contained in complex biological samples (e.g., human blood, plasma, and/or serum). The technology allows a user to analyze heterogeneous populations of nanoparticles derived from complex biological samples that may help characterize, diagnosis, and/or stage disease (e.g., neural diseases such as Alzheimer's).

In contrast to conventional cell-based assays, the described technology uses special fixing and permeabilization protocols that require less exposure time to and/or concentration of potentially toxic chemical agents that can compromise biological structure and/or function. For example, conventional cell-based techniques use fixing protocol that requires 4% PFA and incubation times from 1 hour to overnight (e.g., 16 hours at 4 degrees C.). Times over 1 hour can damage antigens. In contrast, the described technology, in certain embodiments, can effectively detect cargo using 2% PFA with incubation times under 1 hour. In addition, cell and tissue permeabilization protocols require from 0.05 to 1% concentration of Triton™-X or Tween® at incubation times from about 30 minutes to 5 hours. In contrast, the described technology, in certain embodiments, can effectively detect cargo using less than 0.5% Triton™-X and 1 hour incubation times.

Moreover, the described systems and methods detect molecular cargo within particles such as extracellular vesicles (EVs), which contain similar biological information from the parent parental cell but are less complex in structure. The technology can also include a unique blocking step to further increase detection capability of molecular information contained within the EVs.

In certain embodiments of the systems and methods described herein, the imaging system used to detect the extracellular vesicles and the fluorescent molecular cargo is an SP-IRIS system (single-particle interferometric reflectance imaging sensor) as described in the systems and methods of International Publication No. WO2017/136676 titled “Detection of Exosomes Having Surface Markers”, filed on Feb. 3, 2017, the contents of which are hereby incorporated by reference in its entirety. In certain embodiments, substrates used in the SP-IRIS system are modified to enhance label-free and fluorescence imaging of the fluorescently tagged extracellular vesicles bound to a substrate. Examples of substrates that may be used to enhance fluorescence and label-free imaging are presented in International PCT Application No. WO/2019/232321 titled “Compositions, Systems, and Methods for Enhanced Label-Free and Fluorescence—Based Detection of Nanoparticles” filed on May 31, 2019, the contents of which are hereby incorporated by reference in its entirety.

Sensitive detection of molecular cargo within EVs allows therapeutic development. For example, the described technology can be used to analyze molecular cargo within stem-cell derived EVs to help develop natural therapeutic agents in regenerative medicine. Moreover, once active cargo can be determined, EVs can be engineered to package certain therapeutic cargo (e.g., RNA, e.g., microRNA) for therapeutic treatment.

In addition, the described technology serves as a diagnostic and treatment monitoring tool. EVs carry information from the parental cell, which is highly informative to disease detection and/or staging. Surface markers of the EVs reveal what is going on within the cell and/or tumor site. For example, an EV derived from a tumor site can include a population of proteins from the tumor site. In this scenario, the EVs would carry a marker indicative of the tumor and allow a user to test for the disease without having to do a complex biopsy.

In certain embodiments, the described technology uses optical substrates for enhanced detection of molecular cargo. The ability of the described optical substrates to generate enhanced fluorescence signals provides for increased sensitivity and detection of EVs that may facilitate disease detection and monitoring, and other clinical applications. In certain embodiments, the ability of the described systems to simultaneously co-localize both enhanced contrast and fluorescence signals provides for imaging both the outside and inside of particles.

In certain embodiments, a kit contained a premixed solution kit comprising a permeabilization solution, a fixing solution, a blocking solution, and/or a solution with labels.

### A. ISOLATING, LABELING, AND IMAGING VESICLES SUCH AS EXTRACELLULAR VESICLES AND THEIR CARGO

In certain embodiments, the systems and methods described herein can be used to isolate any type of vesicle such as an extracellular vesicle, exosome, liposome, virus, and/or virus-like particle. Moreover, the described systems and methods can be performed using a reflective substrate or other solid phase substrate such as beads (e.g., magnetic beads). It is also noted that fixation, permeabilization, and labeling can be performed prior to or after immobilization/binding to the surface of the selected substrate.

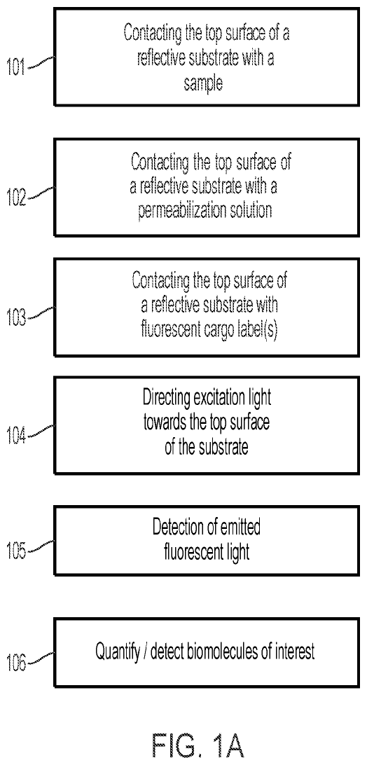

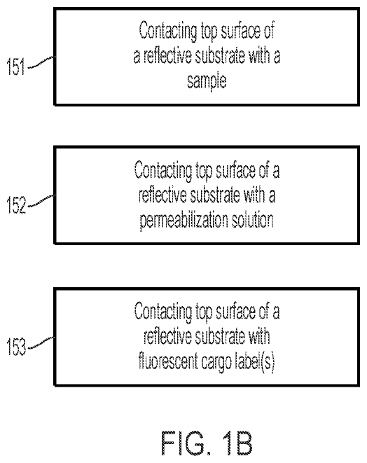

FIG. 1A shows an embodiment of a method of isolating, labeling, and imaging extracellular vesicles and their tagged molecule of interest (e.g., biomolecular cargo). In certain embodiments, the extracellular vesicles are exosomes. In certain embodiments, the biomolecule cargo that is of interest is one or more protein species potentially present within at least a portion of the nanoparticles.

First, a top surface of a reflective substrate is brought into contact with a sample comprising the extracellular vesicles 101, thereby capturing one or more extracellular vesicles present in the sample. In certain embodiments, the sample comprising the extracellular vesicles is a sample obtained from a patient. In certain embodiments, the top surface of the reflective substrate comprises one or more capture agents, wherein each capture agent is specific to a particular target agent associated with at least a portion of the extracellular vesicles. In certain embodiments, the capture agent is an antibody. In certain embodiments, the target agent is an antigen. In certain embodiments, the target agent is expressed on a surface of at least a portion of the extracellular vesicles. Next, the top surface of the reflective substrate is contacted with a permeabilization solution comprising a permeabilization agent 102, thereby permeabilizing the captured extracellular vesicles. In certain embodiments, the permeabilization agent is a detergent. In certain embodiments, the detergent is Triton™. Next, the top surface of the reflective substrate is contacted with one or more fluorescent cargo labels 103, wherein each fluorescent cargo label is specific to one or more biomolecules of interest and comprises a particular fluorescent species, thereby labeling the extracellular vesicles. In certain embodiments, the biomolecule or biomolecules of interest are potentially present in at least a portion of the extracellular vesicles. Light is directed towards the top surface of the reflective substrate 104, thereby exciting the fluorescent cargo labels with which the extracellular vesicles are labeled. In certain embodiments, the top surface of the reflective substrate is imaged at one or more fluorescent wavelengths, each corresponding to an emission wavelength of (a fluorescent species of) a fluorescent cargo label, thereby obtaining one or more fluorescent images, each associated with a particular fluorescent cargo label and biomolecule of interest to which the particular fluorescent cargo label is specific. One or more detectors detect the fluorescent light emitted from the fluorescent cargo label(s) 105 as a result of the excitation by the excitation light. The detected fluorescent light is then used to detect and/or quantify the biomolecules of interest 106 present within the extracellular vesicles.

FIG. 1B shows an embodiment of a method of isolating, permeabilizing, and labeling extracellular vesicles and their biomolecule cargo. In certain embodiments, the extracellular vesicles are nanovesicles. In certain embodiments, the extracellular vesicles are exosomes. In certain embodiments, the biomolecule cargo is one or more protein species potentially present within at least a portion of the nanoparticles. The top surface of a substrate is contacted with a sample comprising the extracellular vesicles 151, thereby capturing one or more extracellular vesicles present in the sample. In certain embodiments, the sample comprising extracellular vesicles is obtained from a subject. In certain embodiments, the top surface of the substrate comprises one or more capture agents, each capture agent being specific to a particular target agent associated with at least a portion of the extracellular vesicles. In certain embodiments, the capture agent is an antibody. In certain embodiments, the target agent is an antigen. In certain embodiments, the particular target agent is expressed on the surface of at least a portion of the extracellular vesicles. Then, the top surface of the substrate is contacted with a permeabilization solution comprising a permeabilization agent 152, thereby permeabilizing the captured extracellular vesicles. In certain embodiments, the permeabilization agent is a detergent. In certain embodiments, the detergent is Triton™. Triton™ is known as: 4-(1,1,3,3-Tetramethylbutyl) phenyl-polyethylene glycol. Triton™ has the chemical formula: C₁₄H₂₂O(C₂H₄O)ₙ, where n is from 9 to 10. The chemical structure of Triton™ is:

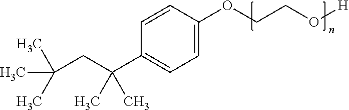

Then, the top surface of the substrate is contacted with a cargo labeling solution 152 comprising one or more fluorescent cargo labels and a blocking agent. In certain embodiments, the blocking agent is bovine serum albumen (BSA). In certain embodiments, the cargo labeling solution comprises the one or more fluorescent cargo labels diluted in a blocking buffer. Each fluorescent cargo label is specific to a particular biomolecule of interest of one or more biomolecules of interest and comprises a particular fluorescent species, thereby labeling the biomolecular cargo within the extracellular vesicles. In certain embodiments, the one or more biomolecules of interest are potentially present in at least a portion of the extracellular vesicles. In certain embodiments, the biomolecular cargo is the one or more biomolecules of interest.

### B. OPTICAL SENSORS AND DETECTION METHODS

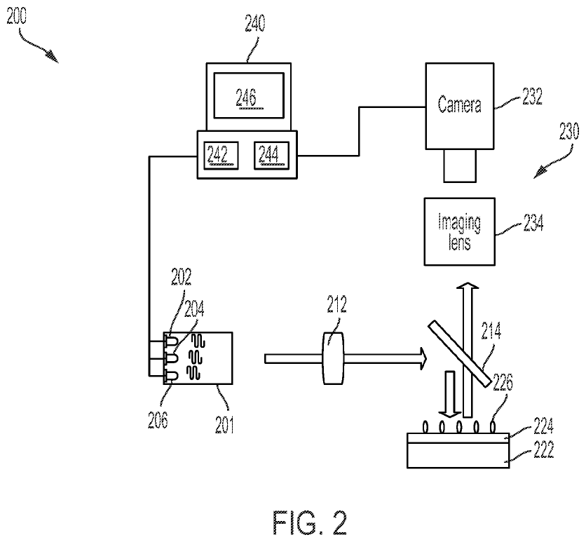

In one or more embodiments, the technology described herein includes apparatus and systems that can detect biomarkers and/or biomolecular cargo (e.g., proteins, e.g., nucleic acids, e.g., DNA, e.g., RNA) within and/or on the surface of vesicles such as exosomes bound to capture agents (e.g., antibodies) on a surface of a substrate. In certain embodiments, the vesicles may be fixed and labeled using the protocols and methods described herein. FIG. 2 illustrates a diagrammatic view of an example imaging system 200 used for imaging vesicles in which the substrates described herein may be used. The system 200 can include an illumination source 201, directing and providing illumination light onto the substrate 222. In certain embodiments as depicted in FIG. 2, the substrate is a reflective substrate 222, having a single oxide layer 223 and the particles (e.g., extracellular vesicles) 226 to be detected, and an imaging system 230 for capturing images of the light reflected by the substrate 222, the oxide layer 224, and the particles 226. In another embodiment, the optical substrate 222 may be a multilayered reflective substrate (not shown) substantially as described herein. The multilayered reflective substrate may comprise a stack of thin, transparent dielectric layers, for example, that is designed for both specific scattering enhancement at a first target wavelength and fluorescence enhancement at a second target wavelength. In certain embodiments, the substrate is mounted and held in place using a mount suitable for the dimensions of the substrate (e.g., a microscope slide mount, a mount for a well plate).

The system 200 can also include a computer system 240 for controlling the illumination source 201 and receiving imaging signals from the imaging system 230. In an embodiment, the illumination source 201 includes incoherent light source (LED) 202 that provides incoherent light in one wavelength having a substantially narrow band of wavelengths. In an embodiment, the illumination source 201 includes a coherent light source (laser). The illumination source may also serve as an excitation source for use in fluorescently tagged particle detection/classification applications (e.g., for the detection of fluorescent labels, e.g., fluorescent cargo labels). In certain embodiments, multiple illumination sources may be utilized. In some embodiments, the illumination source 201 can include three or more coherent or incoherent light sources 202, 204, 206 that produce incoherent light in three different wavelengths. The Light Emitting Diodes (LEDs) or equivalent light sources, each provide incoherent light at one of the plurality of wavelengths. In some embodiments, the illumination source 201 can include an array of illumination elements, including one or more illumination elements providing light at the same wavelength and being arranged in a geometric (e.g., circular or rectangular), random, or spatially displaced array. The light from the illumination source 201 can be directed through a focusing lens 212 and other optical elements (e.g., polarizing lens, filters and light conditioning components, not shown) to a beam splitter 214 that directs the light onto the substrate 222, the oxide layer 224 and the particles 226. Optical components can be provided to condition the light to uniformly illuminate substantially the entire surface of the layered substrate 222. The light reflected by the substrate 222, the oxide layer 224 and the particles 226 can be directed through the beam splitter 214 and imaging lens 234 into a detector (e.g., a camera) 232 to capture images of the substrate surface. In certain embodiments, there may be more than one detector. In certain embodiments, the imaging lens is a high magnification and high resolution objective lens. In certain embodiments, the objective lens is a high magnification objective lens having a magnification ranging from about 4×-100× (e.g., 4×, 10×, 20×, 40×, 60×, 100×). In certain embodiments, the objective lens has a numerical aperture ranging from about 0.1 and about 1.3 (e.g., 0.13, 0.3, 0.5. 0.75, 0.85, 1.25, 1.3). In certain embodiments, light is emitted by a fluorescent label substantially attached to or co-localized with the vesicle (e.g., molecular cargo within the vesicle, molecular biomarkers on the surface of the vesicle). The camera 232 can be, for example, a CCD camera (color or monochromatic) and produce image signals representative of the image based on data corresponding to the illumination light scattered by the particles and/or reflected by the substrate. In another embodiment, the camera 232 can produce image signals representative of the image based on data corresponding to the detected fluorescent light emitted by the fluorescent tags attached to the particles. The image signals can be sent from the camera 232 to the computer system 210 either by a wireless or wired connection.

Computer system 240 can include one or more central processing units (CPUs) and associated memory (including volatile and non-volatile memory, such as, RAM, ROM, flash, optical and magnetic memory) and a display 246 for presenting information to a user. The memory can store one or more computer programs that can be executed by the CPUs to store and process the image data and produce images of the substrate surface. Additional computer programs can be provided for analyzing the image data and the images to detect interference patterns and the particles 226 on the surface of the oxide layer 224 of the substrate 222. Additional computer programs can also provide for analyzing the images of the fluorescent light in conjunction with the image of the particles to enhance imaging of the particles. In other embodiments, the fluorescent light in conjunction with the image of the particles allows for detection of the biomolecular cargo of the vesicles. In certain embodiments, the biomolecular cargo is quantified (e.g., an amount per vesicle, a number of per vesicle, a level per vesicle) (e.g., statistical quantities related to a number and/or amount of cargo) using the data corresponding to the detected fluorescent light.

The computer programs can be executed by the computer to implement a method according to one or more embodiments of the present invention whereby interferometric measurements can be made. The computer programs can control the illumination source 201 comprising one (or more) LED that can be used to illuminate layered substrate. The optical path difference (OPD) between the bottom and top surface causes an interference pattern. The interference patterns can be imaged as intensity variations by the CCD camera 232 across the whole substrate at once.

A variety of software programs and formats can be used to store and/or process optical information obtained via the systems and methods described herein. Any number of data processor structuring formats (e.g., text file, database) can be utilized. By providing optical information in computer-readable form, one can use the optical information in readable form to compare a specific optical profile with the optical information stored within a database of the comparison module. For example, direct comparison of the determined optical information from a given sample can be compared to the control data optical information (e.g., data obtained from a control sample). The comparison made in computer-readable form being the retrieved content from the comparison module, which can be processed by a variety of means.

In an another embodiment, each incoherent light source can be an optical fiber (not shown) that directs the light at the layered substrate 222. Optical components can be provided to condition the light to uniformly illuminate substantially the entire surface of the layered substrate 222.

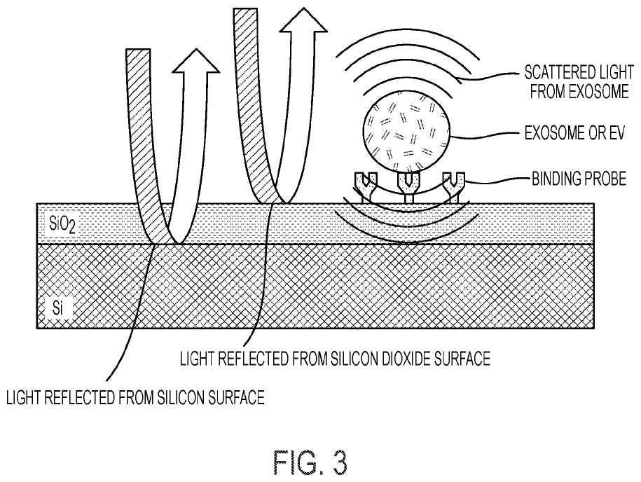

FIG. 3 depicts the interferometric scattering of reflected light upon absorption by vesicles, such as exosomes, wherein said vesicles are bound to capture agents on a surface of a substrate for use in certain embodiments of the methods and systems described herein. In this embodiment, the reflections from the different layers including the silicon surface (Si) and the silicon dioxide surface (SiO₂) interfere with the light reflected from the nanoparticles captured by the capture agents (e.g., antibodies). The interference causes a change in the reflected light, which can be detected by the imaging system as described herein. In particular, a reflectance signature of the incident light is altered by said nanoparticles on a binding layer on the substrate surface to interfere with the light reflected from the Silicon surface and the Silicon Dioxide surface. The imaging system of FIG. 2 detects the interference in the reflection from the extracellular vesicles as compared to reflective properties of the Silicon surface and the Silicon Dioxide and an image processing system comprises a forward model to provide accurate and quantitative sizing of the extracellular vesicles. A preferred embodiment of the imaging device uses a single wavelength (band) of light to measure the interference/mixing of reflected light from the binding layer with the scattered light from the particle (scattering of the light).

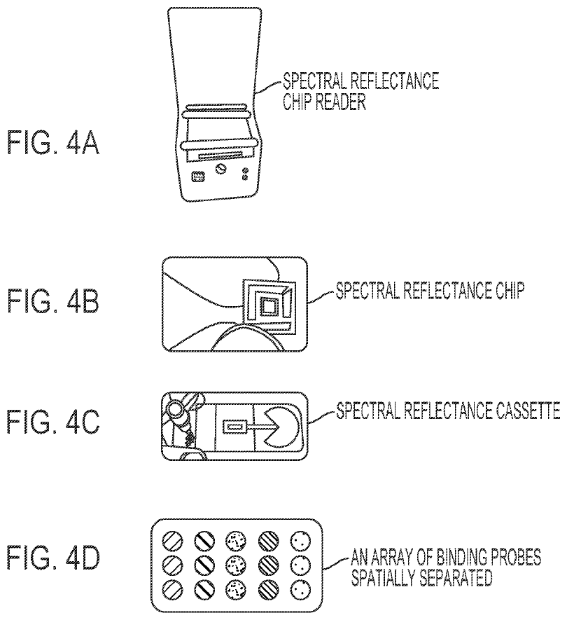

FIG. 4A is an illustrative embodiment of an instrument for the imaging optical substrates as described herein, e.g., for detection of particles (e.g., exosomes), e.g., for the co-localization of exosomes with biomolecular cargo of interest. FIG. 4B is an image of a reflective chip (substrate), as described herein. FIG. 4C is an image of the reflective chip disposed within a microfluidic cassette, which allows flowing of a sample over the substrate. FIG. 4D is an illustration of an array of capture agents (e.g., antibodies) on the substrate, as described herein.

In some embodiments of the exemplified instrument used to image the particles, three or more LEDs with different emission peak wavelengths can be used as the light or excitation source. In some embodiments where more than one incoherent light source is used, the light sources used have a narrow range of wavelengths, and the width between the wavelengths of each individual light source is small. In some embodiments, the light source may also serve as an excitation source for the excitation of fluorescent probes attached to particles. In some embodiments, multiple light sources may be used. In some embodiments, one or more of the light sources is a laser light source.

The use of high-magnification interferometric measurements is an approach to detection of biomolecular targets and particles. The methods and devices described herein provide for imaging of such particles through the use of a high magnification objective lens with a high numerical aperture and placing a spatial filter on the camera's optical axis. The high numerical aperture objective lens will allow imaging at high magnifications and the spatial filter is used to maintain the contrast of the interference cause by the layered substrate by only collecting light from a high angle or a range of angles of incident light. The optical setup described allows for detection of sub-wavelength structures (e.g., of the particles or biomolecular target) without losing contrast or lateral resolution.

Another approach to simplifying the imaging device described herein can be to use a broadband source and a colored CCD camera in which the spectral sampling is done by the camera. Pixels of the camera dedicated for detection of separate colors can be used to extract the intensity of light included in a given spectral band, thus allowing a spectral detection scheme of various wavelengths.

One advantage to the embodiments with an LED light source is that an LED based illumination source allows the imaging device to be more robust and portable, thus allowing field applications. Another advantage is that the light source may serve as an excitation source for a fluorophore species that may be excited at a particular wavelength (band) of light. Moreover, the use of multiple LEDs would allow for the simultaneous or sequential excitation of fluorophores. Another advantage is the high magnification capability of the device. High magnification will allow for the detection of single nanoparticle or biomolecular target on the surface and/or within the nanoparticle (e.g., >a few nm in length or diameter). In some embodiments, a white light source or an RGB LED with a 3CCD or other color camera can be used to capture spectral information at three distinct wavelengths to increase temporal resolution. This is beneficial in studying dynamic biological interactions, for example.

The device as described herein facilitates a method of using an LED illumination source for substrate enhanced detection of particles, such as extracellular vesicles (e.g., exosome biomarkers), in a sample bound to a surface. The LED illumination source may also serve as an excitation source for the detection of fluorescently labeled particles. The device provides a high-throughput spectroscopy method for simultaneously recording a response of an entire substrate surface. The device and methods can be used in any high-throughput application. The device and methods thus provide a platform or a system for high-throughput optical sensing of particles bound to or located substantially close to the surface of a reflective substrate as described herein. The system comprises an illumination source, a reflective substrate, and an imaging device.

In some embodiments the imaging device comprises a camera. The device can be used for multiplexed and dynamic detection of particles (e.g., nanoparticles, [e.g., extracellular vesicles, e.g., exosome biomarkers on a substrate]). Moreover, in some embodiments, the nanoparticles may be labeled with or contain a fluorescent probe (tag) to enhance detection.

Certain embodiments of the device can be described as functional modules, which include computer executable instructions recorded on computer readable media and which cause a computer to perform method steps when executed. The modules can be segregated by function for the sake of clarity. However, it should be understood that the modules need not correspond to discrete blocks of code and the described functions can be carried out by the execution of various code portions stored on various media and executed at various times.

In some embodiments, the device provides a system for detecting and/or classifying particles on a reflective substrate comprising a) a determination module configured to determine optical information, wherein the optical information comprises sampling a least one wavelength using a narrow band light source; b) a storage device configured to store data output from the determination module; c) a comparison module adapted to compare the data stored on the storage device with a control data, the comparison being a retrieved content; and d) a display module for displaying a page of the retrieved content for the user on the client computer, wherein the retrieved content is a light absorption profile of the substrate, wherein a certain light absorption profile is indicative of binding of a particle.

In some embodiments, the imaging device as described herein provides a computer program comprising a computer readable media or memory having computer readable instructions recorded thereon to define software modules including a determination module and a comparison module for implementing a method on a computer, said method comprising a) determining with the determination module optical information, wherein the optical information comprises sampling at least one wavelength using a narrow-band light source; b) storing data output from the determination module; c) comparing with the comparison module the data stored on the storage device with a control data, the comparison being a retrieved content, and d) displaying a page of the retrieved content for the user on the client computer, wherein the retrieved content is a light absorption profile of the solid substrate, wherein a certain light absorption profile is indicative of binding of a particle.

Various modules for determining optical properties include, for example, but are not limited to, microscopes, cameras, interferometers (for measuring the interference properties of light waves), photometers (for measuring light intensity); polarimeters (for measuring dispersion or rotation of polarized light), reflectometers (for measuring the reflectance of a surface or object), refractometers (for measuring refractive index of various materials), spectrometers or monochromators (for generating or measuring a portion of the optical spectrum, for the purpose of chemical or material analysis), autocollimators (used to measure angular deflections), and vertometers (used to determine refractive power of lenses such as glasses, contact lenses and magnifier lens).

As used herein, a cassette is defined as configured to contain a reflective substrate as described herein with a transparent and high-quality imaging window (COP or polycarbonate) with a thin channel of fluid.

As defined herein, a substrate surface can include a “specular reflecting interface.” Such specular reflecting interfaces refer to those surfaces upon which incoming light undergoes “specular reflection,” i.e., the mirror-like reflection of light (or sometimes other kinds of wave) from a surface, in which light from a single incoming direction (a ray) is reflected into a single outgoing direction. Such specular reflecting behavior of a surface, substrate or interface, is described by the law of reflection, which states that the direction of incoming light (the incident ray), and the direction of outgoing light reflected (the reflected ray) make the same angle with respect to the surface normal, thus the angle of incidence equals the angle of reflection; mathematically defined θi=θr. A second defining characteristic of specular reflection is that the incident, normal, and reflected directions are coplanar. Specular reflection can be accurately measured using, for example, a glossmeter. The measurement is based on the refractive index of an object. In some embodiments of the aspects described herein, a specular reflecting interface comprises a substrate having a transparent dielectric layer, for example a layer of Silicon Oxide (SiO₂) on a Silicon substrate. In some embodiments of aspects herein, the layer of Silicon Oxide (SiO₂) has a layer of binding agent for binding to nanoparticles such as an exosome biomarker thereon. In some embodiments, an alternative transparent dielectric layer, such as silicon nitride as well as other coatings can be used as a thin transparent or specular reflecting interface layer.

### C. APPLICATIONS OF THE SENSORS AND METHODS

The ability to detect biological extracellular vesicles, e.g., exosomes, e.g., exosomes comprising an exosome biomarker, e.g., a cell surface biomarker, e.g., biomolecular cargo of the exosome, in a sample is fundamental to understanding of both cell physiology and disease progression, as well as for use in various applications such as the early and rapid detection. Described herein are rapid, sensitive, simple to use, and inexpensive biosensors that are useful for a variety of applications involving the detection of nanoparticles, ranging from research and medical diagnostics, to detection of cancer.

Accordingly, in certain embodiments, the substrates described herein are used to detect binding of extracellular vesicles, e.g., exosomes, e.g., exosomes comprising an exosome biomarker, e.g., a cell surface biomarker, in a sample to a substrate layer, wherein binding of a exosome biomarkers present in a sample contacted with the substrate layer changes an optical path length relative to an optical path length in the absence of the sample, resulting in an interference pattern that is detected and measured by the device and methods described herein. In some embodiment, the sample that contacts the substrate can have a plurality of biomolecular targets, such that multiple extracellular vesicles bind to the substrate layer and are detected by the devices and methods described herein.

The devices and substrates can be used to study one or a number of specific binding interactions in parallel, i.e., multiplex applications. Binding of one or more specific extracellular vesicles in a sample to respective target surfaces can be detected. The substrate is illuminated with light, and if one or more nanoparticle targets in the sample binds one or more capture agents, they will appear in the image as single discrete objects allowing the detection of the individual binding of the nanoparticle to the capture agent. In embodiments where a substrate surface comprises an array of one or more distinct target locations comprising one or more specific targets, then the interference pattern is detected from each distinct location of the substrate. Accordingly in certain embodiments, the vesicles are fixed using the procedures described herein. The fixed vesicles are then labeled using a fluorescent label (e.g., a fluorescent cargo label) to identify the presence and/or absence of biomolecular cargo of interest.

In some embodiments, a variety of specific target molecules can be immobilized in an array format onto the substrate surface. The substrate is then contacted with a test sample of interest comprising potential nanoparticle targets, such as exosomes. Only the exosomes that specifically bind to the capture agents are bound to the surface of the substrate. For high-throughput applications, biosensors can be arranged in an array of arrays, wherein several substrates comprising an array of specific binding molecules targets on the substrate surface are arranged in an array.

Accordingly, the devices and substrates can be used to detect binding of one or more of a plurality of nanoparticle targets present in a sample to a biosensor substrate layer having one or more of a plurality of capture agents attached to the substrate layer. For example, one or more specific immobilized molecules can be arranged in an array of one or more distinct locations on the surface of the substrate layer. The one or more distinct locations can define microarray spots of about 50-500 microns, or about 150-200 microns in diameter.

For example, the binding agents can be immobilized on a layered substrate surface that has a spectral reflectance signature that is altered upon immobilization of said nanoparticles on a binding layer on the substrate surface. In particular, as will be described herein, the image processing system detects the extracellular vesicles a function of the change in reflective properties of the substrate and an image processing system comprises a forward model to provide accurate and quantitative sizing of the extracellular vesicles. In particular, a preferred embodiment of the device uses a single wavelength (band) of light to measure the interference/mixing of reflected light from the binding layer with the scattered light from the particle (scattering of the light). As extracellular vesicles bind to the binding layer, the scattered light from these objects interfere with the reflected light from the substrate surface making the extracellular vesicles observable on an imaging device as discrete objects (dots). The substrate is illuminated with one (or more) wavelengths of light, and if one or more extracellular vesicle objects in the sample binds with the binding layer, the nanoparticle target will appear in the image as single discrete objects, thereby allowing the detection of the individual binding of the nanoparticle targets as well as the quantitative sizing of the extracellular vesicles. The apparatus allows for the simultaneous imaging of the entire field of view of a surface for high-throughput applications. The apparatus and method has several advantages such as low-cost, high-throughput, rapid and portable detection.

Also described herein are methods of use of the device for the detection of a variety of biomolecular targets. In some aspects, the devices and methods described herein provide a high-throughput method for simultaneously recording a response of an entire substrate surface, comprising sampling at least one wavelength using a light source providing incoherent light, and imaging the reflected or transmitted light using an imaging device. The device can include a light-emitting diode (LEDs) as the illumination source for interferometric principles of detection. Interferometric measurements can provide desired sensitivity and resolution using optical path length differences (OPD).

Accordingly, described herein are devices and methods for substrate enhanced detection of binding of molecules or nanoparticles or extracellular vesicles such as exosomes to a surface of a substrate. The device samples the reflectance spectrum by illuminating the substrate with at least one wavelength of light, using, for example, an LEDs and recording the reflectance by an imaging device, such as a 2-D arrayed pixel camera. In this way, the reflectance spectrum for the whole field-of-view is recorded simultaneously. Using this device and method, high-throughput microarray imaging can be accomplished. The present technology can also provide high-magnification imaging for detection of biomolecular nanoparticle targets in the 30 nm to a few (2-3) microns in range. Such high-magnification detection can be used, for example, for the detection of a single particle on a capture surface.

The instrument and process provide a high-throughput spectroscopy technique where sampling at least one wavelength is realized by using a narrowband light sources, such as an LED, and the reflected or transmitted light is imaged to an imaging device, such as a monochromatic CCD camera, thus allowing the response of the entire imaged surface to be recorded simultaneously. The microarray can be fabricated on a layered substrate (for example: anywhere from a few nm of SiO₂ up to 100 nm of SiO₂ layered on a Si wafer). A preferred embodiment includes a green LED light source (535 nm) and 100 nm oxide of SiO₂ layered on a Si wafer. A second preferred embodiment includes an ultraviolet LED light source (420 nm) and 60 nm oxide of SiO₂ layered on a Si wafer. A third preferred embodiment, for use when imaging in complex media, includes an ultraviolet LED light source (420 nm) and 30-to-60 nm oxide of SiO₂ layered on a Si wafer.

In some embodiments three or more LEDs with different emission peak wavelengths can be used as the light source. In some embodiments where more than one incoherent light source is used, the light sources used have a narrow range of wavelength, and the width between the wavelengths of each individual light source is small. In some embodiments, one or two light sources are used.

In some embodiments described herein, the microarray or binding agent is fabricated on a layered substrate comprising anywhere from a few nanometers to 100 nm of SiO₂ layered on a Si wafer. In some embodiments, the microarray or binding agent is fabricated on a layered substrate comprising 95-100 nm of SiO₂ layered on a Si wafer. In some embodiments, the microarray or binding agent is fabricated on a layered substrate comprising 30-60 nm of SiO₂ layered on a Si wafer. A preferred embodiment includes a green LED light source (near 535 nm) and 100 nm oxide of SiO₂ layered on a Si wafer. A second preferred embodiment includes an ultraviolet LED light source (near 420 nm) and 60 nm oxide of SiO₂ layered on a Si wafer. A third preferred embodiment, for use when imaging in complex media, includes an ultraviolet LED light source (near 420 nm) and 30-to-60 nm oxide of SiO₂ layered on a Si wafer. The devices and methods described herein, can be used, in part, for high magnification interferometric measurements, for example, but not limited to, detecting extracellular vesicles, such as an exosome biomarker for a cancer, in a given sample.

Examples of sensors and methods that can be used with the described optical substrates include, but are not limited to, are described by Daaboul et al., in International Publication No. WO2017/136676 titled “Detection of Exosomes Having Surface Markers”, filed on Feb. 3, 2017, the contents of which are hereby incorporated by reference in their entirety.

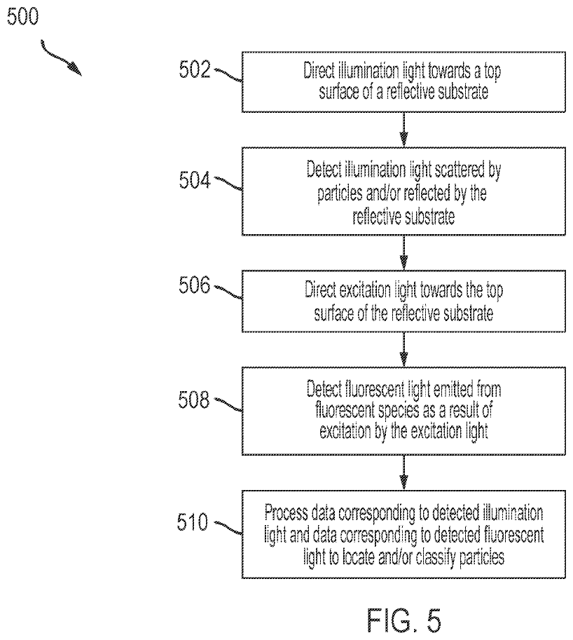

Turning to FIG. 5, in certain embodiments, the reflective substrates described herein can be used in methods for detecting and/or classifying particles (e.g., nanoparticles, e.g., nanovesicles, e.g., exosomes, e.g., biomolecular cargo) located substantially in a target plane above and in proximity to a top surface of a reflective substrate via simultaneous detection of fluorescence and single-particle, label-free scattering. The simultaneous detection of fluorescence in one or more channels and a label-free scattering signal allows for the co-localization of vesicles (e.g., exosomes) along with interior and/or exterior markers. In particular, the methods described herein regarding the permeabilization and labeling of biomolecular cargo and/or markers within and/or on the vesicles are particularly advantageous to identify and/or classify particles of interest. For example in one embodiment, multiple types of biomolecular cargo of a vesicle may each be fluorescently labeled using the methods described herein. In one example, each cargo type may be labeled with a unique antibody, each unique antibody having a different fluorophore attached. The fluorophores in different channels may then be co-localized with the scatter-free signal to classify and/or quantify features of the vesicles. For example, co-localization of fluorophores may help to determine the origin/type of vesicle (e.g., cancer type of origin, cell type of origin), size of the vesicle, a shape factor of the vesicle, cargo type. In certain embodiments, the classification of the vesicles may further include a determination of spurious label-free or fluorescent signals (e.g., mis-identified vesicles, e.g., non-specific binding of tags to a surface). In certain embodiments, the identified vesicle locations as co-localized with the fluorophores may be used to detect and/or quantify a portion of the one or more particular biomolecules of interest [e.g., by identifying vesicle locations from which fluorescent emission is detected, e.g., thereby determining a fraction, prevalence, etc. of expression of one or more particular biomolecules of interest].

FIG. 5 shows an example process 500 for such a method. In one step 502, illumination light is directed to the top surface of the reflective substrate. Illumination light that is scattered by the particles and/or reflected by the reflective substrate is detected (e.g., to obtain a scattering image) 504. In another step 506, excitation light, is directed towards the top surface of the reflective substrate, to excite fluorescent cargo labels on a surface of and/or within the vesicles. In certain embodiments, the fluorescent light emitted by the fluorescent species may then be detected (e.g., to obtain a fluorescence image) 508 using one or more detectors. Data corresponding to (i) the detected portion of the illumination light that is scattered by the particles and/or reflected by the reflective substrate (e.g., the scattering image) and (ii) the detected fluorescent light (e.g., the fluorescence image) can be used to locate and/or classify at least a portion of the particles may then be processed to detect and/or classify the particles 510. In certain embodiments, the biomolecules of interest (e.g., the biomolecular cargo) are quantified. In certain instances, co-localization of the fluorescent and scattered light images (e.g., the “label-free” image) allows for characterization of the size of the vesicles, the type of vesicles, the identification of cargo within vesicles, and/or the removal of “spurious” non-specific signals.

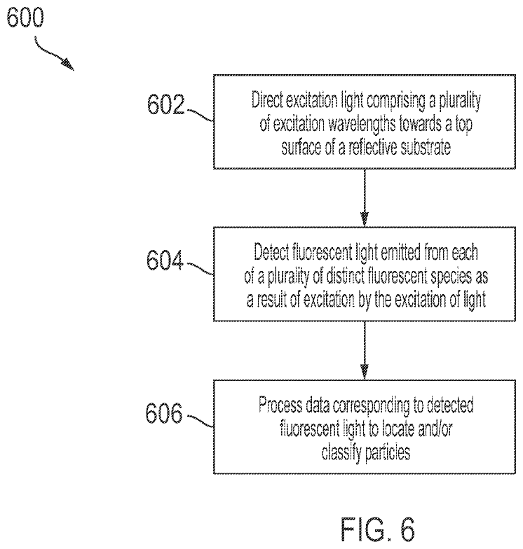

The reflective substrates described herein may also be used for enhancement of fluorescence from multiple distinct fluorescent species (e.g., fluorescent antibodies targeted to an antigen) located within and/or at a surface of particles. Such substrates may be used in methods for detecting and/or classifying particles (e.g., nanoparticles; e.g., nanovesicles; e.g., exosomes) via detection of enhanced fluorescence from a plurality of distinct fluorescent species within and/or on a surface of the particles. FIG. 6 shows an example process 600 for detecting and/or classifying particles via detection of enhanced fluorescence in this manner. In one step 602, excitation light comprising a plurality of wavelengths is directed to a top surface of the reflective substrate. The excitation light excites various distinct fluorescent species, having multiple distinct excitation bands, with which the biomolecular cargo contained within/on the surface of particles are labeled. Fluorescent light emitted by the plurality of fluorescent species in response to excitation by the excitation light may then be detected 604 (e.g., to obtain multiple fluorescence images, each corresponding to a distinct fluorescent species). Data corresponding to the detected fluorescent light (e.g., the multiple fluorescence images) may then be processed to locate and/or classify particles 606.

In certain embodiments, the approaches described herein include methods for labeling internal and/or surface biomarkers and/or biomolecular cargo of particles, such as nanovesicles, exosomes, and the like, with fluorescent species and for contacting the particles with the reflective substrate (sensor chip). In one approach, for example, the particles (e.g., nanovesicles) can be incubated with a fluorophore-probe complex comprising the fluorescent species before contacting with the sensor chip. In another approach, for example, the particles (e.g., nanovesicles) can be labeled with the fluorophore-probe complex after being captured on the chip.

i. Data Processing

The systems and methods described herein also include unique data processing approaches that provide for counting and characterization of individual particles (e.g., vesicles, molecular cargo) by measuring intensity profiles of corresponding particle image features within fluorescence and/or scattering images (e.g., obtained using the reflective substrates described herein). For example, as presented herein, the co-localization of fluorescent and label-free signals provides a particular, unique advantage in classifying and/or identifying vesicles (e.g., exosomes) and their biomolecular cargo. In label free images, particles may appear as image features (e.g., spots; e.g., diffraction limited spots; e.g., having an airy disc shape) that are relatively bright or dark in comparison with respect to their surrounds. Measurement of intensities of such particle image features in label free images may include determining a peak particle feature intensity, measurement of an entire “airy disk” envelope of the particle image feature, and other methods of quantifying the intensity of the particle image feature.

The images taken of the optical substrate with the attached particles may be processed to detect individual particles (e.g., vesicles, e.g., exosomes) and provide intensity information about them. Particle detection may be accomplished via a template matching stage where particle image features are identified based on knowledge that particles that are below the diffraction limit of the microscope will form an “airy disk” given by a point spread function of the microscope. This a priori knowledge of the form that the particles will take in the image allows detection and then quantification of the number of particles as well as their intensity, which can be used to provide sizing information.

When particle intensity features are detected within multiple images, such as fluorescence and scattering images, particle intensity features in different images that correspond to the same particle can be matched to each other, providing for co-localization of the particles in the various images. For example, particle intensity features in a scattering signal and a fluorescence signal at one wavelength may be performed. In another embodiment, wherein multiple fluorescence images are obtained, co-localization of particle intensity features in the multiple fluorescence images allows for co-localization of multiple fluorescent markers used to obtain the different fluorescence images.

In one example, co-localization of particle image features in a label-free (scattering) image and in a fluorescence image obtained via detection of Cy3 emission of a tag on a target (e.g., biomolecular cargo) is performed. In one example, co-localization of particle image features in two fluorescence images is performed, wherein a first fluorescence image is obtained via detection of Cy3 emission of a tag attached to a first target and a second fluorescence image is obtained via detection of Cy5 emission of a tag attached to a second (or the same) target. In one example, co-localization of particle image features in a label-free (scattering) image and two fluorescence images obtained via detection of Cy3 and Cy5 emission is performed.

In certain embodiments, the co-localization of fluorescent and label-free signals obtained from particles may be classified as being indicative of any one of or a combination of the following: (i) the absence of a preselected cancer, (ii) the presence of a preselected cancer, (iii) the presence of a non-cancerous disorder of a preselected tissue, or (iv), the presence of a preselected pre-cancerous lesion of the preselected tissue. In certain embodiments, the subject is classified as being at an elevated risk of (i) not having the preselected cancer, (ii) having the preselected cancer, (iii) having the non-cancerous disorder of the preselected tissue, or (iv) having the preselected pre-cancerous lesion of the preselected tissue. In certain embodiments, the method comprises monitoring and/or evaluating the progress or state of the preselected cancer.

ii. Fluorescent Labeling Approaches

Fluorophores can be attached to a probe to indicate the presence of a molecular target on and/or within a particle (e.g., a nanovesicle, e.g., an exosome). The fluorophore can be an organic dye, fluorescent protein, a substrate of an enzyme. The fluorophore may have excitation and emission band in the visible spectrum.

In certain embodiments, fluorescent species comprise nucleic acid dye that can be used to stain for RNA and DNA in and/or on the particles (e.g., exosome). In certain embodiments, a molecular beacon can be used to detect a specific sequence on a nucleic acid.

Lipid dyes can be used to probe the composition of the lipid membrane of vesicles. These lipid dyes can be used to detect composition and understand the biogenesis of the vesicle. Furthermore, by staining the lipid membrane of the vesicle the signal correlates with the surface area of the vesicles. Accordingly, lipid staining with a lipid dye can be an orthogonal measurement to confirm the size of the vesicle and/or it can allow sizing of vesicles when light scatter is too low to detect. Other dyes include 5-(and-6)-Carboxyfluorescein Diacetate Succinimidyl Ester and Carboxyfluorescein succinimidyl ester.

iii. Classifying and/or Detecting Vesicles

In certain embodiments, the reflective substrates described herein can be used in methods for detecting and/or classifying particles (e.g., nanoparticles, e.g., nanovesicles, e.g., exosomes, e.g., biomolecular cargo) located substantially in a target plane above and in proximity to a top surface of a reflective substrate via co-localization of fluorescence and single-particle scattering. In one step, illumination light is directed to the top surface of the reflective substrate. Illumination light that is scattered by particles (e.g., exosomes) and/or reflected by the reflective substrate is detected (e.g., to obtain a scattering image). In another step, excitation light, is directed towards the top surface of the reflective substrate, to excite fluorescent species at a surface of and/or within the particles (e.g., biomolecular cargo, e.g., biomarkers). Fluorescent light emitted by the fluorescent species may then be detected (e.g., to obtain a fluorescence image). Data corresponding to (i) the detected portion of the illumination light that is scattered by the particles and/or reflected by the reflective substrate (e.g., the scattering image) and (ii) the detected fluorescent light (e.g., the fluorescence image) and to locate and/or classify at least a portion of the particles may then be processed to locate and/or classify the particles.

The reflective substrates described herein may also be used for enhancement of fluorescence from multiple distinct fluorescent species located within and/or at a surface of particles. Such substrates may be used in methods for detecting and/or classifying particles (e.g., nanoparticles; e.g., nanovesicles; e.g., exosomes) via detection of enhanced fluorescence from a plurality of distinct fluorescent species within and/or on a surface of the particles. In one step, excitation light comprising a plurality of wavelengths is directed to a top surface of the reflective substrate. The excitation light excites various distinct fluorescent species, having multiple distinct excitation bands, with which the particles (e.g., nanoparticles; e.g., nanovesicles; e.g., exosomes) are labeled. In certain embodiments, the fluorescent species are bound to a biomarker and/or biomolecular cargo within and/or on the surface of the particle. Fluorescent light emitted by the plurality of fluorescent species in response to excitation by the excitation light may then be detected (e.g., to obtain multiple fluorescence images, each corresponding to a distinct fluorescent species). Data corresponding to the detected fluorescent light (e.g., the multiple fluorescence images) may then be processed to locate and/or classify the particles.

In certain embodiments, the approaches described herein include methods for labeling particles, such as nanovesicles, exosomes, and the like, with fluorescent species and for contacting the particles with a reflective substrate (e.g., a sensor chip). In one approach, for example, the particles (e.g., nanovesicles) can be incubated with a fluorophore-probe complex comprising the fluorescent species before contacting with the sensor chip. In another approach, for example, the particles (e.g., nanovesicles) can be labeled with the fluorophore-probe complex after being captured on the chip.

In certain embodiments, the described optical substrates are used in combination with the systems and methods described by Daaboul et al., in International PCT Application No. PCT/US2017/016434, filed on Feb. 3, 2017, the content of which is hereby incorporated by reference in its entirety. In certain embodiments, the methods include the isolation of circulating extracellular vesicles (e.g., cancer derived extracellular vesicles) (e.g., exosomes) from a sample obtained from a subject using a substrate as described herein by contacting the surface of the substrate with a sample and detecting the extracellular vesicles bound to the surface of the substrate. In certain embodiments, the circulating extracellular vesicles are bound to the surface of the substrate using one or more binding agents (e.g., as described herein) (e.g., an antibody, a nucleic acid, a polypeptide, and/or an aptamer). In certain embodiments, the method comprises evaluating the level (e.g., quantity, e.g., number, e.g., concentration) of extracellular vesicles in the sample. In certain embodiments, the method comprises evaluating the number of extracellular vesicles bound to the substrate or a predetermined portion thereof. In certain embodiments, the method comprises determining a size (e.g., diameter, volume) of one or more extracellular vesicles bound to the substrate.

### D. ENHANCED SUBSTRATES FOR LABEL-FREE AND/OR FLUORESCENT BASED DETECTION OF NANOPARTICLES

In certain embodiments, the described systems and methods are used in combination with the compositions, systems, and methods for enhanced label-free and fluorescence-based detection of nanoparticles such as exosomes described by Daaboul et al., in Provisional Application No. 62/714,204, filed on Aug. 3, 2018 and in International PCT Application No. PCT/US2019/034831 filed on May 31, 2019, the contents of which are hereby incorporated by reference in its entirety.

In certain embodiments, the above mentioned compositions, systems, and methods for enhanced label-free and fluorescence based detection of nanoparticles are related to optical substrates that (1) enhance a fluorescence signal emitted by a fluorophore and/or (2) enhance “contrast” signal (“label-free” signal) that comprises scattered signal intensity over substrate reflectance at a non-fluorescent wavelength. In certain embodiments, the optical substrate comprises a thin, transparent, layer (e.g., a dielectric layer, e.g., an oxide layer). In alternative embodiments, the optical substrate comprises a stack of thin, transparent dielectric layers, for example, that is designed for both specific scattering enhancement at a first target wavelength and fluorescence enhancement at a second target wavelength. The ability of the described optical substrates to co-localize both enhanced contrast and fluorescence signals simultaneously provides for increased sensitivity and detection of nanoparticles, such as extracellular vesicles (e.g., exosomes), that may facilitate disease detection and monitoring, and other clinical applications.

In certain embodiments, optical substrates that provide for increased sample excitation and/or fluorescence emission also serve to increase scattering contrast at a separate wavelengths. Scattering contrast is measured by exciting dipoles at a non-fluorescent wavelength and measuring the ratio of particle intensity to background intensity (see Eq. 1):

$${E}_{ref} = {E}_{i n c} + r {E}_{i n c} ; Contrast = \frac{\vert {E}_{r e f} + {E}_{s c a} {\vert}^{2}}{\vert {E}_{r e f} {\vert}^{2}} \qquad ( Eq . 1 )$$

Enhancing scattering contrast requires a different optimization than fluorescence enhancement, which includes designing a surface so that the intensity maximum of the standing wave interference pattern is in the same plane as the fluorophore. Contrast, however, is affected by the scattered electric field from the nanoparticle interfering with the incident electric field and the reflected field off the substrate to produce the intensity picked up by a detector such as a CCD array. The specific radiation pattern for the scatter can be calculated by making a spherical approximation and, for example, using Mie theory or Rayleigh scattering theory depending on the radius of the particle in question.

In fluorescence, the excitation wavelength is different than the emission wavelength, meaning that the emitted electromagnetic (EM) radiation from the fluorophore does not interfere with the excitation light. This is a fundamentally different interaction than when trying to size and characterize particles and other biological matter using a contrast measurement. Fluorescent enhancement can be approximated by maximizing local electric field in the plane of the fluorophore across its excitation bandwidth and simultaneously increasing reflectance across its emission bandwidth to collect more of the forward scattered light. Assuming back-scattered and forward-scattered light are equivalent in amplitude, this reduces to:

$$F = \frac{{E}_{e n h a n c e}}{2} ( 1 + R ) = ( {\int }_{{λ}_{1}}^{λ} {}^{2} \frac{\vert {E}_{i n c} [ λ ] ( 1 + r [ λ ] ) {\vert}^{2}}{2} d λ ) * ( 1 + {\int }_{{λ}_{3}}^{{λ}_{4}} R [ λ ] d λ )$$

For a standard glass slide and the Cy3 fluorophore, the fluorescence factor, F, is approximately 0.34, with an electromagnetic field enhancement factor, E_{enhance} of 0.66 for the Cy3 fluorophore on a glass slide.

In certain embodiments, a maximum enhancement for fluorescent intensity is 8-fold. A 4-fold enhancement may be obtained from the electric field strength intensity enhancement and a 2-fold enhancement from the collection of all forward scattered light reflected off the substrate. This number is considered F, where F for a glass slide is 0.66. As illustrated in the equation development below, a single (60 nm thick oxide layer) produces an enhanced fluorescence image signal relative to a glass slide:

F=E(1+R)

F_{glass}=0.65(1+0.0351)=0.66 for Cy3

F_{1layer}(60 nm)=1.49(1+0.19)=1.78.

i. Multilayer Film Stacks

In certain embodiments, substrates comprise an optical interference coating corresponding to a multi-layer stack. In certain embodiments, the multi-layer stack comprises three-layers. In certain embodiments, the multi-layer stack comprises alternating low and high refractive index layers. Examples of various multi-layer stacks comprising alternating high and low refractive index layers are shown below (a base layer, corresponding to a Si substrate, and the ambient, air layer, are also shown):

Si—SiO₂-Metal-SiO₂-Air

Si—SiO₂—Ni—SiO₂-Air

Si—SiO₂—Si—SiO₂-Air

Si—SiO₂—TiO₂—SiO₂-Air

In certain embodiments, a top layer is a biologically receptive material (e.g., SiO₂, Si₃N₄). In certain embodiments, a middle layer is or comprises a thin layer of metal or high refractive index material (e.g., a metal; e.g., Ni; e.g., Si; e.g., TiO₂). In certain embodiments, the optical interference film comprises one or more layers of dielectric materials such as, but not limited to, SiO₂, TiO₂, Si, Ta₂O₅, HfO₂, ZrO₂, MgO, Si₃N₄, MgF₂ and YF₃. In certain embodiments, each layer is less than 130 nm (e.g., quarter-wave of 750 nm for SiO₂). High refractive index layers may have refractive indices between 2.3 and 4. Low refractive index layers may have refractive indices between 1.1 and 1.7.

ii. Wavelength Ranges for Fluorescence and Scattering Enhancement

Fluorescence and scattering enhancement as described herein may be achieved at a variety of wavelengths using the approaches described herein. In certain embodiments, enhancement is obtained at wavelengths in a visible region (e.g., for wavelengths ranging from 400 nm to 750 nm). Various “scattering” or label-free target center wavelength in the visible region are listed below:

1. From about 400 to about 450 nm (UV)

2. From about 460 to about 500 nm (Blue)

3. From about 520 to about 560 nm (Green)

4. From about 640 to about 680 nm (Red)

5. From about 710 to about 750 nm (Deep Red)

In certain embodiments, measurements of scattering signals use illumination light having shorter wavelengths is used to detect smaller particles. The approaches used herein, however, may be used to engineer a substrate for use with a longer wavelength to prevent bleaching and damaging of biological particles or other effects for example.

Another example involves detection of fluorescence from fluorescent species with which particle labels, wherein the fluorescent species can be excited at a traditional label-free wavelength (e.g., 420 nm). In certain embodiments, rather than detect scattering a the traditional label-free (e.g., scattering) wavelength, it is preferable to detect the scattered signal using a longer wavelength, for example so as to prevent photo bleaching and then detect the fluorescence traditionally. Accordingly, a substrate designed to enhance scattering signal at a long wavelength outside the excitation range (e.g., Red) and to enhance fluorescence where the particle can be excited/emits (e.g., Blue) can be used.

### E. EXAMPLES

### Example 1: Methods for Detecting Biomolecular Cargo Inside of Exosomes

The present example describes an exemplary method for detecting biomarkers (e.g., protein, e.g., nucleic acids, e.g., DNA, e.g., RNA) inside of and/or on the surface of vesicles using a labeled antibody.

Examples of biomarkers described in this Example include ALIX (ALG-2-interacting protein X), Syntenin, and TSG 101 (tumor susceptibility gene 101, a protein), and GFP (fluorescent proteins).

Immobilization of Exosomes on a Substrate

First, a sample comprising particles such as exosomes is incubated on a substrate as described herein (e.g., a reflective substrate, e.g., an optical substrate, e.g., the ExoView™ Chip) or such as one described in U.S. Provisional Application No. 62/714,204 entitled “COMPOSITIONS, SYSTEMS, AND METHODS FOR ENHANCED LABEL-FREE AND FLUORESCENCE-BASED DETECTION OF NANOPARTICLES” filed on Aug. 3, 2018, the contents of which is hereby incorporated by reference in its entirety. In this example, the optical substrate is functionalized using antibodies to capture and isolate particles such as exosomes from a sample. An example of a functionalized optical substrate is described in International Application No. PCT/US17/16434 entitled “DETECTION OF EXOSOMES HAVING SURFACE MARKERS” filed on Feb. 3, 2017, the contents of which is hereby incorporated by reference in its entirety. In addition, FIG. 3 is an illustrative example of exosomes captured on an exemplary functionalized surface.

Immobilizing vesicles onto a solid surface (e.g., a substrate described herein, beads) followed by fixing improves the stability of the structure when permeabilized. In detection methodologies that do not require immobilization of particles (e.g., flow cytometry), but instead perform detection in liquid phase, vesicles (e.g., exosomes) that are fixed and permeabilized do not maintain their structure. The vesicles will, as a result, solubilize. Solubilization will result in detection of molecular components (e.g., biomolecular cargo) as individual entities, and not as a collection of molecules belonging to and/or associated with individual vesicles.

After immobilization of the vesicles onto the substrate, the chip is then washed to remove any unbound exosomes. The bound exosomes then undergo a labeling protocol such as the following.

Overview of Cargo Labeling Protocol

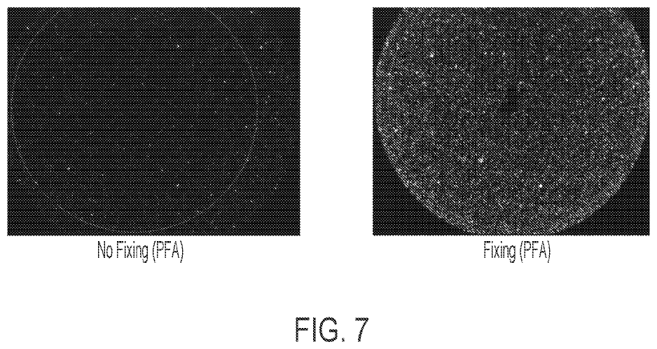

First, the bound exosomes are fixed with 2% paraformaldehyde (PFA) in phosphate buffer saline (PBS) for about 10 minutes. FIG. 7 shows the effect fixation has on fluorescent labeling. Fixation at this step significantly improves fluorescent labeling of cargo later in the process over labeling without fixation. Fixing crosslinks the components of the exosomes, making it more rigid and resistant to losing its structure when permeabilized.

Optimization of the fixing and permeabilization for vesicles is a difficult process. Incorrect experimental parameters, as discussed herein, will lead to damage to the antigens and/or vesicles. Significantly longer fixation times (e.g., 1 hr or more) can damage the antigens (e.g., biomolecular cargo) within and/or on the surface of the exosomes. In addition, it has been found that, for example, EDC (1-ethyl-3-(3-dimethylaminopropyl) carbodiimide) based crosslinkers are not suitable for such a crosslinking as it would damage the exosomes.

Aldehyde fixation (e.g., using formaldehyde, glutaraldehyde, etc.) is a technique that crosslinks proteins found in the cytoskeleton and elements of the cytoskeleton to each other. Chemical modification of proteins using aldehydes can destroy antigens. This problem is not apparent when fixing cells. However, when aldehydes are used to fix tissues, the method requires very long treatments to allow sample penetration. Long treatments alter protein structure. Avoiding prolonged fixation times in both cells and tissues is advised when using aldehydes where it is possible to do so. In certain embodiments, after fixation with aldehydes a step called a “quenching” step is employed. Quenching reduces autofluorescence as aldehyde fixatives react with amines and proteins to generate fluorescent products. In certain embodiments, quenching may be performed with a solution comprising a quenching agent (e.g., glycine).

Permeabilization of the membrane of the vesicle after fixation allows access to the luminal and internal molecular cargo of the vesicle. Accordingly, in one embodiment, the exosomes are then permeabilized with 0.05% Triton™ in PBS (PBST) from about 2 minutes to 10 minutes. Higher concentrations of Triton™ (e.g., greater than 1%) and significantly longer incubation times (e.g., greater than 1 hr) leads to damage to the exosomes and/or antigens of interest.

The chip is then incubated with a fluorogenic probe (e.g., for about 1 hour at room temperature, e.g., overnight at 4° C.) in 10% bovine serum albumin (BSA) in PBST at a concentration of about 0.05 μg/mL. However, concentrations of fluorogenic probe may range up to 10 μg/mL. The chip (e.g., as depicted in FIG. 4B) is then washed with buffer, dried, and read on a detection system (e.g., the ExoView™ Reader, e.g., as depicted in FIG. 4A) such as the one described in U.S. Provisional Application No. 62/714,204 entitled “COMPOSITIONS, SYSTEMS, AND METHODS FOR ENHANCED LABEL-FREE AND FLUORESCENCE-BASED DETECTION OF NANOPARTICLES” filed on Aug. 3, 2018, the content of which is hereby incorporated by reference in its entirety.

During the fixation and permeabilization process, drying of the chip is avoided in order to prevent the dehydration and/or drying of proteins. Dehydration of proteins results in significant, measurable conformational changes as observed using Fourier-transform infrared spectroscopy and resolution-enhancement techniques. As a consequence, these conformational changes are at least partially irreversible. In certain instances, proteins are observed to denature and aggregate. The pK value (i.e., the disassociation constant), which is used to describe antigen-capture agent binding strength, can be perturbed by various factors (e.g., protein folding, dehydration, charge-charge interactions, charge-dipole interactions).

In certain embodiments, organic solvents may be used to fix vesicles. Organic solvents (e.g., methanol, acetone, ethanol) do not alter target proteins covalently. Organic solvents precipitate proteins out of solution. Organic solvents cause cells to flatten or collapse, resulting in a protein shell. The shell makes penetration of certain cellular structures (e.g., the nucleus, mitochondria) more difficult. In addition, organic solvents also remove lipid-linked proteins. However fixing cells using organic solvents (e.g., methanol, ethanol, acetone) provides advantages for use with certain antibodies. For example, organic solvents are useful when using monoclonal antibodies that bind to only one target antigen naturally buried within internal protein structures.

Exemplary Steps of Cargo Labeling Assay Protocol

A sample comprising extracellular vesicles (EVs) or exosomes is incubated on an optical substrate (e.g., NanoView™ chip, e.g., FIGS. 4A and 4D) (hereinafter “chip”) having antibodies serving as capture agents spotted on the surface of the chip. Note: the antibody type is selected based on the antigens expressed by the extracellular vesicles (EVs) or exosomes.

Step 1—Washing Unbound Particles

Chips are washed using “HEPES buffer” comprising: 50 mM HEPES, 150 mM sodium chloride, 5 mM EDTA, 5 mM EGTA and 0.05% Tween® 20, three times for 5 mins each time using a shaker at 500 rpm. The addition of Tween® 20 allows unbound/non-specifically bound particles to be removed easier from the antibody. Tween® 20 is also known as polysorbate 20. Tween®20 has the chemical formula C₅₈H₁₁₄O₂₆. Tween®20 has the chemical structure:

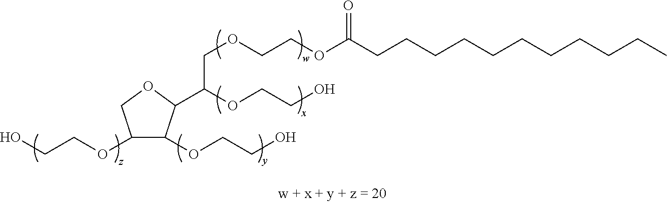

Procedure
1) After incubation of the sample on the chip overnight: 1 mL of HEPES buffer was added to the well, and shaken for 5 mins.
2) 750 μL of buffer was removed, and 750 μL of HEPES buffer was then added (this process is repeated a total of 3 times).
3) After the last HEPES buffer wash, 750 μL of solution volume was removed, so that there was 250 μL of solution left (volume left in the well) (e.g., to preserve the integrity of the EVs).

Step 2—Fixing the Chip with Attached EVs (Fixing EVs)

After removing unbound particles, chips comprising immobilized EVs were fixed (or crosslinked) using paraformaldehyde (PFA-methanol free) diluted in PBS (pH 7 to pH 7.4). In certain embodiments, over-fixation may cause damage to an antigen expressed by the EV.

Procedure
4) 250 μL of 4% PFA was added to a well having 250 μL of HEPES buffer to bring the final concentration of PFA to 2%.
5) Chips were incubated with fixing solution at room temperature for 10 mins.

Step 3—Washing PFA Off the Chip

The purpose of this step is to remove excess PFA and preserve the antigen as much as possible.

Procedure
6) 500 μL of HEPES buffer is added, making the total volume on the well 1 mL. Subsequently, 750 μL of solution is removed, and another 750 μL of the HEPES buffer added. These steps are done to perform buffer exchange, and dilute PFA to avoid antigen damage.
7) Chips are shaken for 5 mins. Thereafter, 750 μL of solution is then removed, and 750 μL of HEPES buffer is added. This process is repeated three times.
8) After the last HEPES buffer wash, 750 μL of solution is removed, so that 250 μL of solution is left in the well (e.g., to avoid drying of the chip).

Step 4—Permeabilization of EVs

The purpose of this step is to allow internalization of the antibody in later steps. Triton™-X, a detergent, was used as a main permeabilization agent (e.g., wherein the concentration of Triton™-X is within a range from about 0.05% to about 1%). Timing must be taken into consideration as the membrane from the EVs can become damaged or rupture, and inadvertently release biomolecular cargo (e.g., proteins, DNA, RNA). The timing described by this exemplary protocol was optimized to a time suitable for using a concentration of 0.05% Triton™ (or Triton™-X) in PBS. Note that incubation time can vary based on the concentration of Triton™ that is used. Triton™-X concentration was chosen to maintain the integrity of the membrane of the EVs.

Procedure
9) 250 μL of a solution of 0.1% Triton™-X was added to the well that already contained 250 μL of HEPES buffer, so that the total concentration of Triton™ in the well is 0.05%. Chips were incubated at room temperature (no shaking) for 10 mins.
10) 500 μL of HEPES buffer were added to the well making the total volume 1 mL. Subsequently, 750 μL of solution was removed, and another 750 μL of HEPES buffer were added. This step is done to perform a buffer exchange and avoid membrane damage as fast as possible.
11) The chips were washed for 5 mins. 750 μL of solution was then removed, and another 750 μL of HEPES buffer added. This step is repeated three times.

Step 5—Immunolabeling

Procedure
12) While the chips were being washed, the antibody was diluted in 10% BSA in HEPES buffer. Starting dilution is about 1:5000 (e.g., given the initial concentration of the antibody is 0.5 mg/mL). In certain embodiments, the range of the initial antibody concentrations is from about 0.05 mg/mL-5 mg/mL. The starting dilution is then varied accordingly. Up to three antibodies labeled with different fluorophores can be used in a cocktail.
13) Once done washing the chips, all of the volume in the well was removed and 250 μL of antibody (or cocktail of antibodies) in 10% BSA solution were added.
14) The chips were then incubated for 1 h at room temperature, in the absence of shaking.

Step 6—Washing the Unbound Antibody and Getting the Chip Ready to be Scanned

The addition of washing steps at this stage removes unbound/non-specifically bound labeled antibody to EVs.

Procedure
15) After incubation, 750 μL of HEPES buffer is added, making the total volume in the well 1 mL. Subsequently, 750 μL of solution is removed, and another 750 μL is added. This step dilutes the antibody to avoid any unspecific binding caused by excess time of incubation at room temperature.
16) The chips are then shaken for 5 mins at 500 rpm at room temperature.
17) 750 μL of solution is then removed and another 750 μL of HEPES buffer is added. These steps are repeated three times.
18) On the final wash, 750 μL of solution is removed and 750 μL of HEPES buffer without Tween 20® are added. This step removes detergent from the chips.
19) After washing with HEPES buffer without Tween®, 750 μL of solution was removed and 750 μL of diH2O is added to remove the salts from the HEPES buffer.
20) The chips are then transferred individually to a 6-cm Petri dish with diH2O, without allowing chip to dry.
The chips are then removed from water at a 45-degree angle allowing the water to slide off uniformly thus removing excess water, and then air dried.

Experimental Results

Cell culture-derived extracellular vesicles (EVs) (e.g., exosomes) in a human plasma sample were incubated on a chip. The chip comprises antibody capture areas to bind EVs to the surface of the chip. After incubation of the sample on the chip, a permeabilization protocol (such as a protocol described herein) or no permeabilization was followed. Chips that were not permeabilized were exposed to 2% PFA for fixing for 10 minutes and rinsed in PBS. Chips were contacted with 0.5% BSA for 1 hour to block unspotted areas of the chip (“blocking solution”). The chips were then contacted with an antibody solution diluted in the blocking solution for 1 hour. Chips that were permeabilized were exposed to 2% PFA for fixing for 10 minutes. Chips were then permeabilized in 0.05% Triton™ for 10 minutes. Chips were contacted with 0.5% BSA (“blocking solution”) for 1 hour to block unspotted areas of the chip. The chips were then contacted with an antibody solution diluted in the blocking solution for 1 hour.

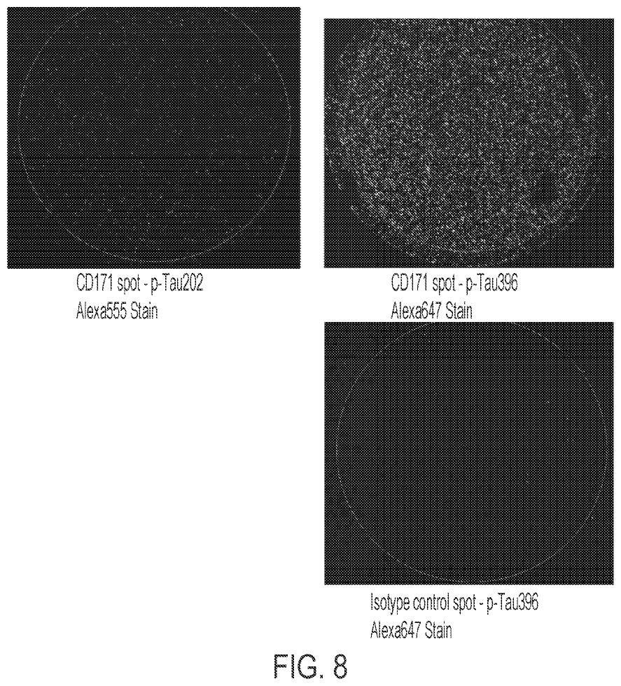

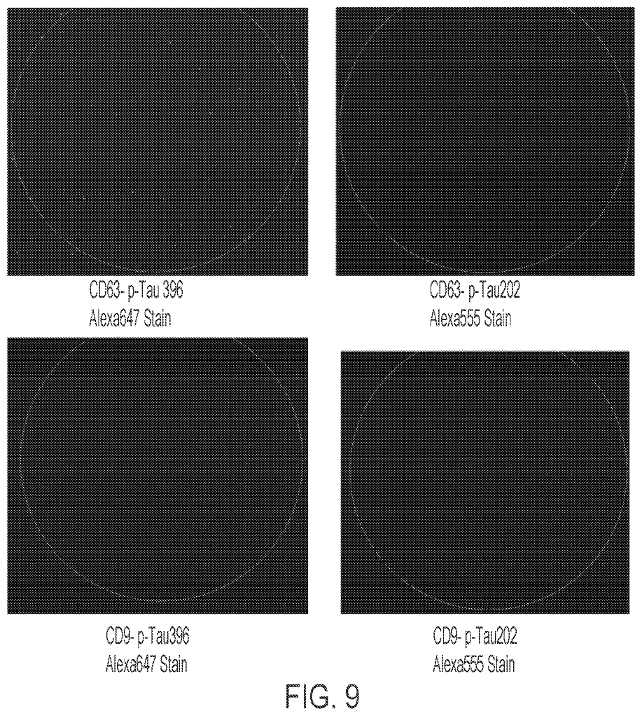

Plasma was included on the chip and EVs were captured on anti-CD81, CD63, CD9, CD171, and isotype control spots. The label-free interferometric imaging modality shows that EV capture occurred on anti-CD63, CD9, and CD171 spots (FIG. 8). Next, the fluorescence imaging modality was used to determine whether these captured particles contained two different p-Tau proteins, p-Tau Ser202 and p-Tau Ser396 (FIG. 9). Chips were incubated with anti-p-Tau Ser202-Alexa555 and anti-p-Tau 396-Alexa647 according to a permeabilization protocol described herein. Interestingly, p-Tau is seen to be expressed on CD171 positive exosomes, which are enriched for neuronal-derived EVs in plasma (FIG. 8). Moreover, even though thousands of vesicles were captured by the CD63 and CD9 spots, these spots did not stain for p-Tau (FIG. 9). These results demonstrate enhanced fluorescence detection, e.g., combined with enhanced label-free detection, can be used to study the heterogeneity of EV from complex samples like human plasma.

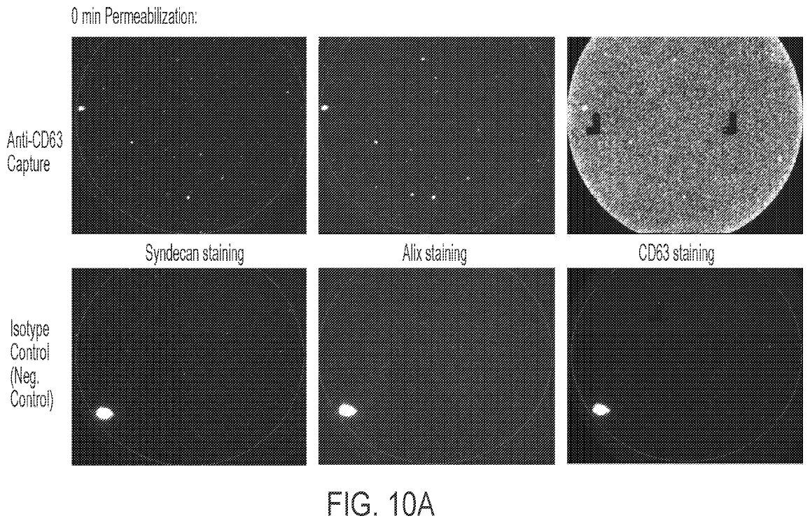

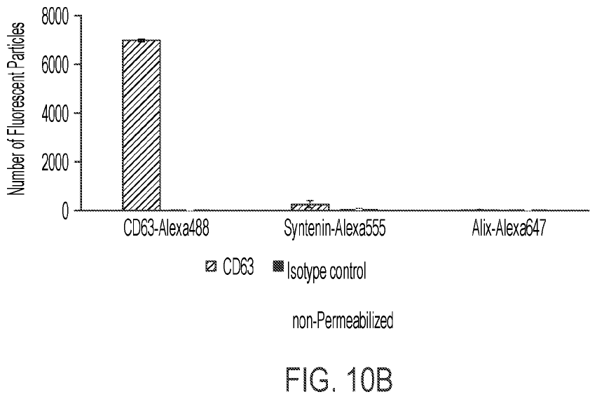

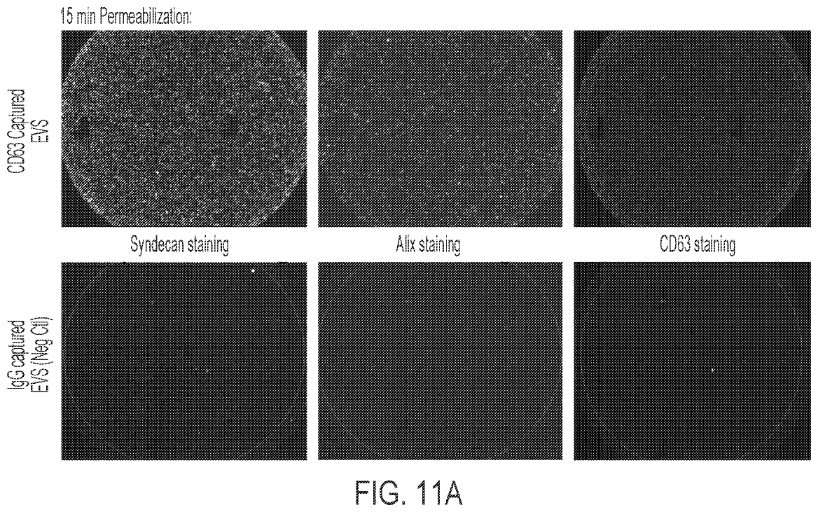

FIG. 10A shows fluorescence images of extracellular vesicles (EVs) bound to an optical substrate (or chip) using an anti-CD63 antibody. The captured EVs were labeled with anti-CD63 (green; right images), Syntenin (yellow; left images), and ALIX (red; middle images). CD63 is a surface marker while Syntenin and ALIX are internal markers. The bottom panel is a series of images taken on an isotype control area of the chip and acts as a negative control.

FIG. 10B shows a graph depicting a number of fluorescent particles identified in each of the fluorescence channels for identifying CD63 capture and a negative control. The data demonstrates that, without permeabilization, particles are only detected in the green channel that detects labeled CD63 (which is a surface marker on the particles). Internal markers and negative control showed no labeling.

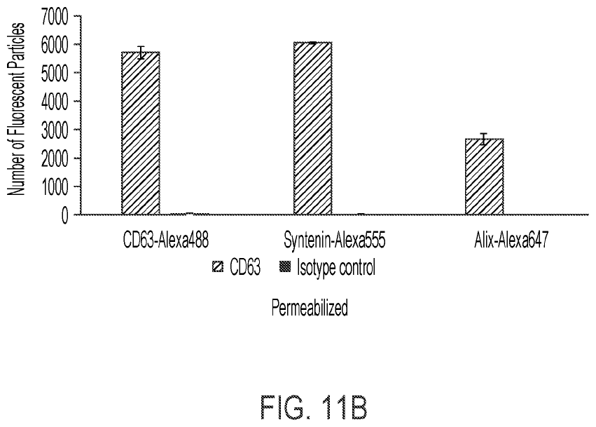

FIG. 11A shows fluorescence images of extracellular vesicles (EVs) bound to an optical substrate (or chip) using an anti-CD63 antibody. After performing a permeabilization protocol described herein on the captured EVs, the EVs were labeled with anti-CD63 (green), Syntenin (yellow; left images), and ALIX (red; middle images). CD63 is a surface marker while Syntenin and ALIX are internal markers. The bottom panel is images taken on the isotype control area of the chip and acts as a negative control. The images show fluorescence signal on internal markers after permeabilization.

FIG. 11B shows a graph depicting a number of fluorescent particles identified in each of the fluorescence channels for identifying CD63 capture and a negative control. The data demonstrates that, with permeabilization, particles are detected in the green channel that detects labeled CD63 (which is a surface marker). Internal markers are detected in the yellow and red channels. The negative control showed no labeling.

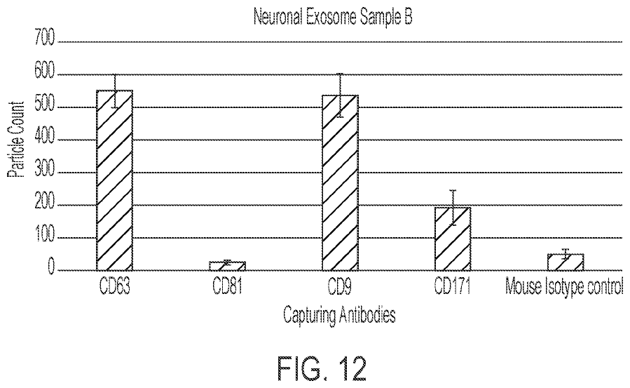

FIG. 12 shows a graph depicting capture of extracellular vesicles (EVs) from human plasma on a chip comprising immobilized antibodies against CD63, CD81, CD9, CD171, and an isotype control. The imaging system described herein (SP-IRIS imaging) shows that a significant amount of particle/EVs were captured on CD63, CD9, and CD171. The imaging measurements show that a significant number of the extracellular vesicles were captured using each of different antibodies (i.e., anti-CD63, anti-CD81, anti-CD9, anti-CD171 antibodies). The extracellular vesicles were quantified and identified using a label-free method.

FIG. 8 shows images depicting a permeabilization protocol described herein and labeling vesicles with antibodies against p-Tau Ser202 (Alexa555, Green) and p-Tau Ser396 (Alexa647, Red). The EVs bound to CD171 are positive for both p-tau Ser202 and p-Tau Ser396, while the isotype negative control showed no labeling.

FIG. 9 shows images depicting that after using a permeabilization protocol described herein and labeling vesicles with antibodies against p-Tau Ser202 (Green) and p-tau Ser396 (Red), the EVs bound to CD63 and CD9 are not positive to p-tau Ser202 and p-tau Ser396.

### Example 2: Determining Parameters for Cargo Labeling and Permeabilization of Extracellular Vesicles

The present example presents results showing how process parameters, in particular concentration and incubation times of permeabilization and fixation agents, are determined in order to provide for effective fixing, permeabilization, and labeling of extracellular vesicles and their cargo. As described herein, developing fixing, permeabilization, and labeling procedures appropriate for extracellular vesicles is non-trivial. Conventional protocols, e.g., that might be applicable to cells, are not viable for extracellular vesicles. These protocols use concentrations of particular agents, such as cross-linking and fixing agents, and incubation times that may damage extracellular vesicle samples and the particular associated biomolecules that they carry and/or express on their surfaces. Preserving integrity of extracellular vesicles and associated biomolecules is key to imaging and sensing techniques such as those described herein, which aim to detect intact vesicles along with the bimolecular cargo they carry. The results shown herein demonstrate development of protocols that accomplish this goal, and can be used to provide for imaging of intact extracellular vesicles and their cargo.

FIG. 7 depicts images depicting the importance of sample fixation. Samples were incubated in the presence (right) or absence (left) of PFA. Incubation was done for 10 minutes at room temperature. A permeabilization protocol was performed with and without a fixation/crosslinking agent. The fixation/crosslinking provided a significant improvement in the labeling of internal molecular cargo inside the EVs as shown in the image which demonstrate a higher level of fluorescence signal.

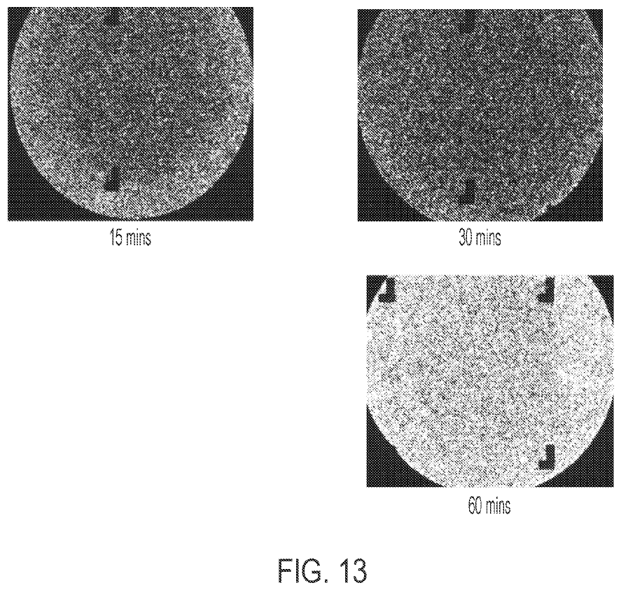

FIG. 13 depicts images related to antibody incubation time. A cargo labeling step was performed for different lengths of time (15, 30, 60 minutes). Sufficient labeling is seen at 15 minutes. However, fluorescence corresponding to labeling of internal cargo continued to increase with time. The samples show a higher degree of binding at 60 minutes than at 15 minutes.

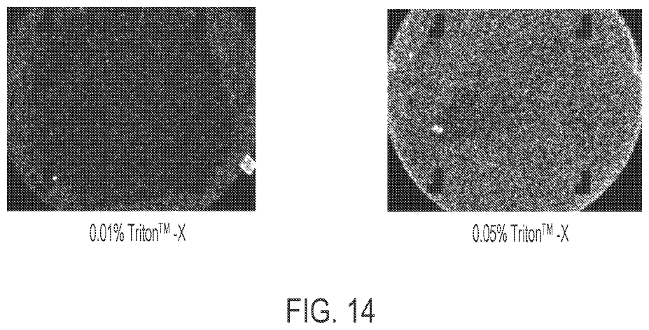

FIG. 14 depicts images related to effects of Triton™-X concentration on permeabilization. Permeabilization was improved when Triton™-X concentration increase from 0.01% to 0.05%. Signal is still possible to measure at higher concentration up to 1% Triton™-X. However, to protect the integrity of the antigen, 0.05% was chosen as the concentration used in the experiments described herein.

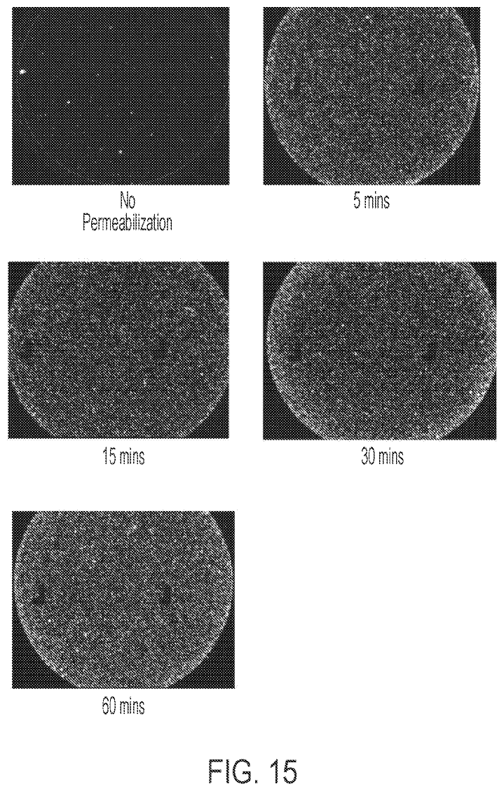

FIG. 15 shows images depicting the effect of incubation time on permeabilization (no permeabilization, 5 minutes, 15 minutes, 30 minutes, 60 minutes). After selecting 0.05% concentration of Triton™-X, permeabilization time was varied at this concentration. It was found that 5 minutes to 60 minutes incubation time did not substantially affect detectable signal. Therefore, to protect the membrane of the EVs, a 10 minute incubation time was selected for the experiments described herein.

### Example 3: Detection of Syntenin in “Raw” and SEC Purified Human Plasma

The present example demonstrates the detection of Syntenin, a protein, associated with extracellular vesicles in purified human plasma samples and “raw” human plasma samples.

Purified human plasma samples were obtained by further processing the human plasma using size exclusion chromatography (SEC). Size exclusion chromatography is a technique to purify extracellular vesicles from biological samples or cell conditioned media.

Samples were then incubated on chips (e.g., a chip as described herein) having anti-CD63, anti-CD9, or anti-CD81 capture antibodies on the surface of the chip. mIgG was used as an isotype control. The capture antibodies are capture agents, which immobilize extracellular vesicles onto the surface of the chip. The chips were then washed to remove unbound material. Extracellular vesicles bound to the chip via capture antibodies remained bound to the chip. The extracellular vesicles were then fixed, permeabilized, and labeled according to the methods described herein. The samples were fluorescently labeled using an anti-Syntenin antibody conjugated to CF®555 using the cargo labeling protocols described herein.

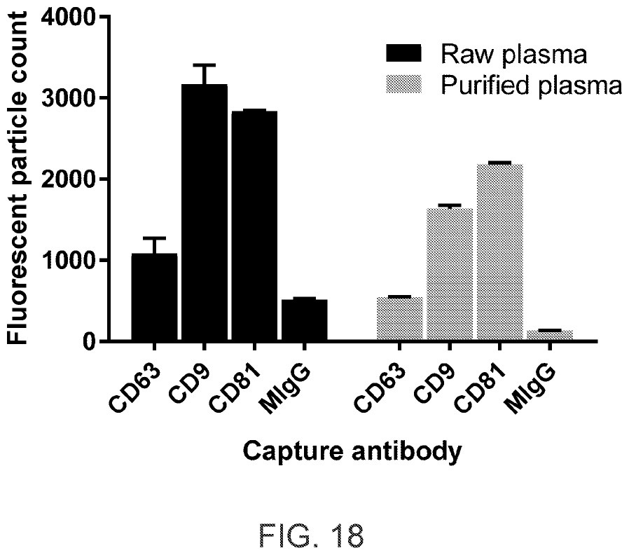

FIG. 18 shows a graph of number of fluorescently tagged particles (i.e., extracellular vesicles) captured on the surface of a substrate (e.g., a chip as described herein) using anti-CD63, anti-CD9, and anti-CD8 antibodies as capture agents in the raw and purified human samples. MIgG is used as a negative/isotype control. The number of fluorescently tagged extracellular vesicles in purified plasma is corrected for the dilution introduced when using SEC to purify the extracellular vesicles.

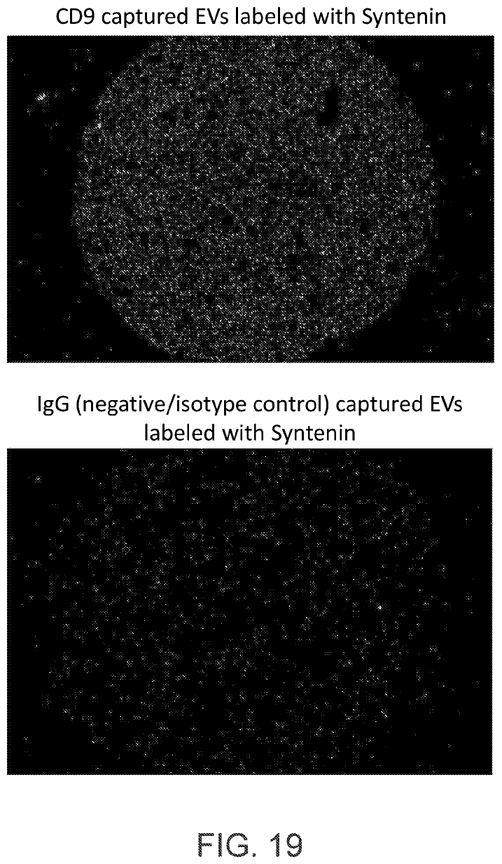

FIG. 19 is a series of images acquired from raw plasma samples. In the top panel, extracellular vesicles captured on a substrate with anti-CD9 antibodies are fluorescently labeled using an anti-Syntenin antibody conjugated to CF®555 using the cargo labeling protocols described herein. On the bottom, an isotype control area of the chip is shown, which acts as a negative control. The images show the presence of Syntenin in EVs captured by the CD9 antibody, and the absence of Syntenin in EVs captured by IgG.

### Example 4: Detection of Syntenin in “Raw” and SEC Purified Human Plasma

The present example demonstrates the detection of Syntenin, a protein, associated with extracellular vesicles in mouse cerebrospinal fluid and cell culture media obtained from mouse cell lines bEnd3 and NIH 3T3.

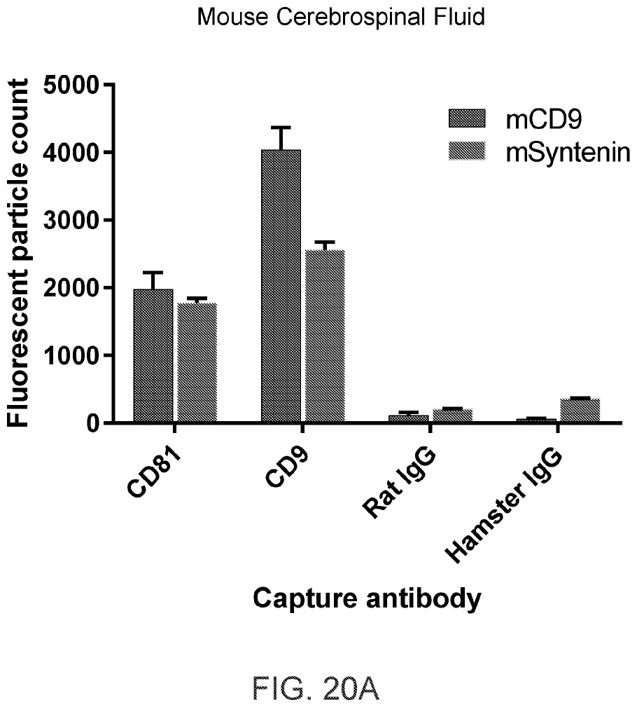

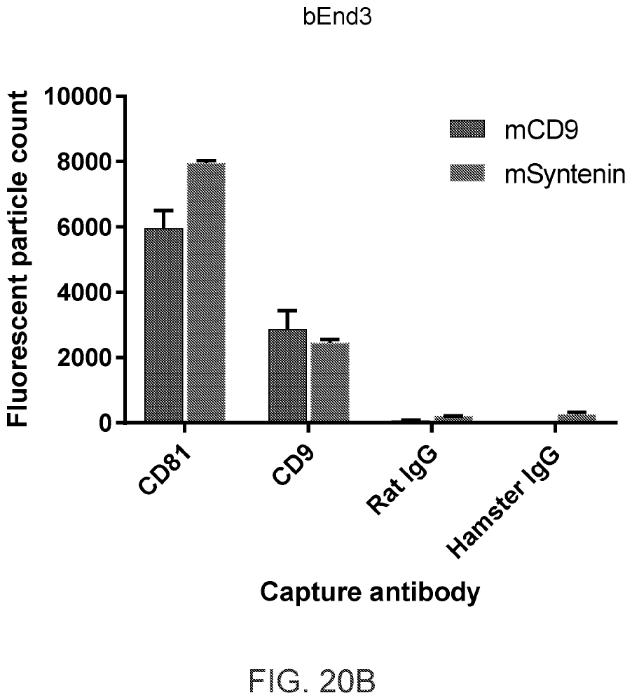

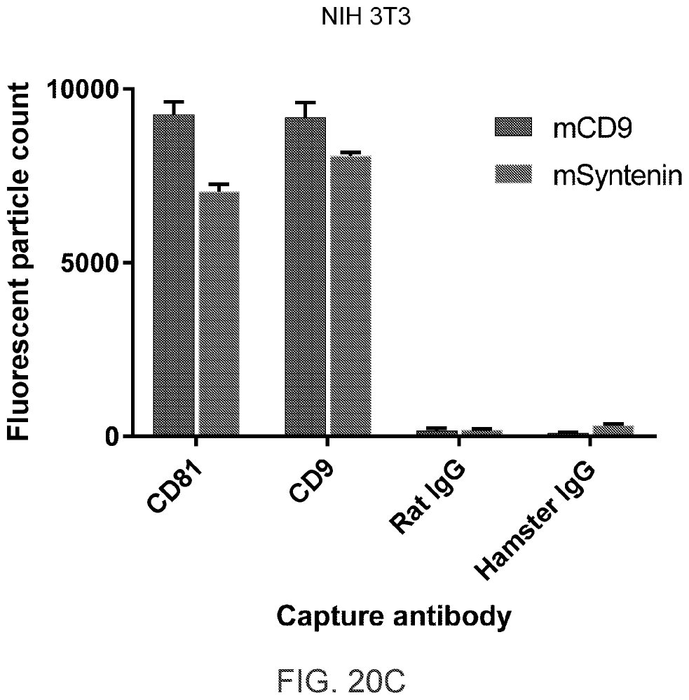

Using the methods and systems described herein, exosomes derived from mouse cerebrospinal fluid (FIG. 20A), bEnd3 cells (mouse brain endothelial cells) (FIG. 20B), and NIH 3T3 cells (mouse fibroblasts) (FIG. 20C) were incubated on chips (e.g., a chip as described herein) having anti-CD9 or anti-CD81 capture antibodies on the surface of the chip. Rat IgG and hamster IgG were used as isotype controls. The capture antibodies are capture agents, which immobilize extracellular vesicles onto the surface of the chip. The chips were then washed to remove unbound material. Extracellular vesicles bound to the chip via capture antibodies remained bound to the chip. The extracellular vesicles were then fixed, permeabilized, and labeled according to the methods described herein. The samples were immunolabeled using anti-mSyntenin and anti-mCD9 antibodies conjugated to fluorophores.

The graphs of FIGS. 20A-20C demonstrate the presence of Sytenin in cell line derived EVs. CD9 immunolabeling was used to demonstrate that internal and external proteins can be detected at the same time, and that the identified EVs were co-expressing known exosome markers.

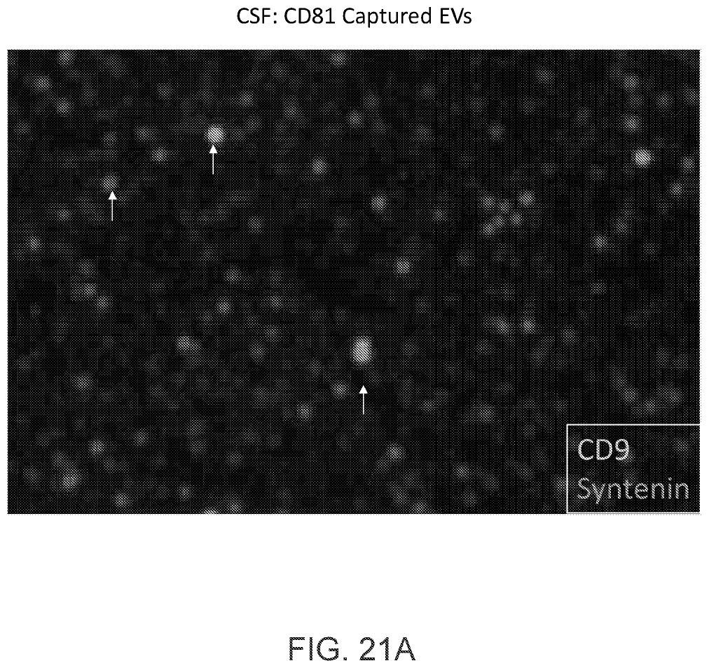

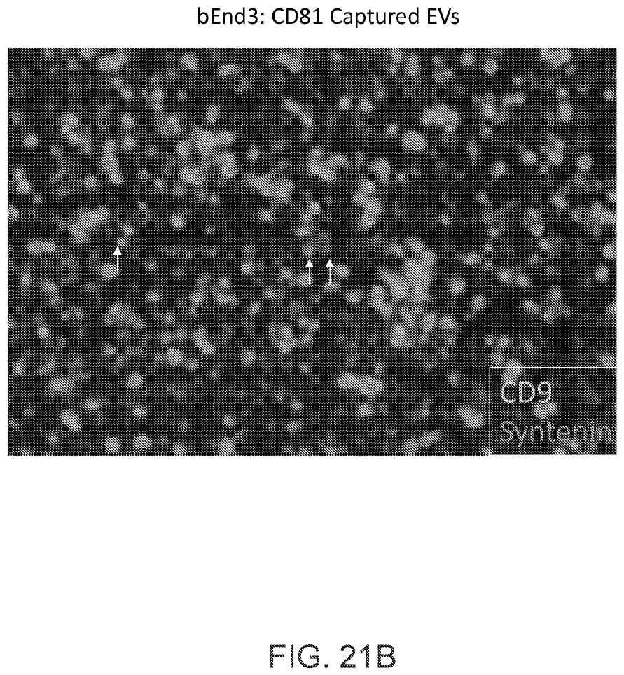

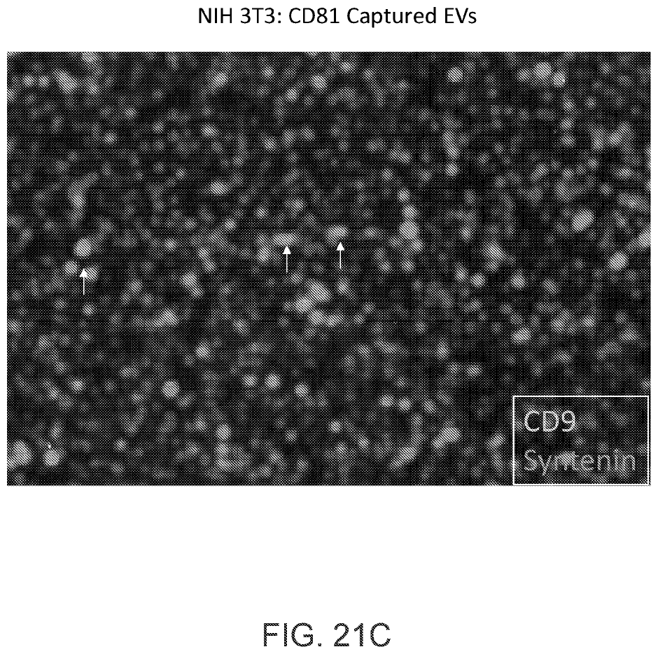

FIG. 21A is a fluorescent image of CD81 captured EVs derived from CSF on a substrate. CSF EVs were captured by CD81 and immunolabeled with anti-mCD9 and anti-mSyntenin. Arrows show EVs that are both CD9 and Syntenin positive. The data show that the immunolabeling is detecting vesicles based on the presence of CD9. The data also show that the EVs carry Syntenin. Arrows show a few points where CD9 and Syntenin signal is co-localized.

FIG. 21B is a fluorescent image of CD81 captured EVs derived from bEnd3 cells. bEnd3 EVs were captured by CD81 and immunolabeled with anti-mCD9 and anti-mSyntenin. Arrows show EVs that are both CD9 and Syntenin positive. The data show that the immunolabeling is detecting vesicles based on the presence of CD9. The data also show that the EVs carry Syntenin. Arrows show a few points where CD9 and Syntenin signal is co-localized.

FIG. 21C is a fluorescent image of CD81 captured EVs derived from NIH3T3 cells. NIH3T3 EVs were captured by CD81 and immunolabeled with anti-mCD9 and anti-mSyntenin. Arrows show EVs that are both CD9 and Syntenin positive. The data show that the immunolabeling is detecting vesicles based on the presence of CD9. The data also show that the EVs carry Syntenin. Arrows show a few points where CD9 and Syntenin signal is co-localized.

### Example 5: Label Free and Fluorescence Signal Co-Localization

The present example demonstrates the co-localization of label free and fluorescence signals on the same extracellular vesicle using the methods and systems described herein.

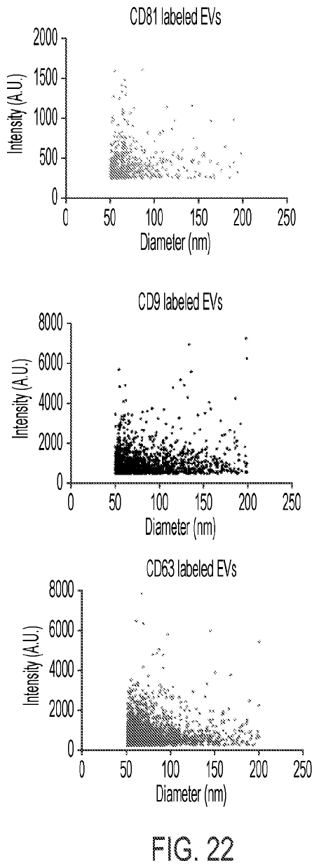

FIG. 22 shows a set of graphs of data determined from images of fluorescently immunolabeled EVs. The EVs were captured on a substrate using anti-CD63 antibody. Later, the captured EVs were fluorescently labeled (e.g., fluorescently immunolabeled) with anti-CD9, anti-CD81, and anti-CD63. In certain embodiments, the procedure is carried out using a non-cargo labeling protocol (e.g., without fixation and permeabilization). The data show the combination of SP-IRIS and fluorescence data. SP-IRIS imaging data are used to derive the diameter of the vesicles, while fluorescence data are used to derive the intensity data.

Table 1 below shows example of data of particle size (i.e., diameter) versus fluorescence intensity of Syntenin labeling in vesicles.

TABLE 1
Fluorescence Intensity vs. Particle Size

| Diameter (nm) | Intensity (A.U.) |
|---|---|
| 51.59 | 660 |
| 75.58 | 293 |
| 131.1 | 503 |
| 58.38 | 254 |

### F. VESICLES, CAPTURE AND LABELING AGENTS, AND BIOMOLECULES OF INTEREST

Particles are bound to the surface of the substrates by the interaction of the particles with capture agents on the surface of the substrates. In certain embodiments, the particles are vesicles (e.g., exosomes, extracellular vesicles, liposomes, viruses, and virus-like particles) as described herein. Bound vesicles may subsequently be imaged, and in certain embodiments, labeled using one or more labeling agents (e.g., fluorescent labeling agents) to observe the biomolecular cargo of interest co-located within and/or on the surface of the vesicles. In certain embodiments, the biomolecular cargo contained within and/or present on the surface of such vesicles may be, without limitation, proteins, nucleic acids (e.g., DNA, e.g., RNA), peptides, and other biomolecules of interest.

i. Vesicles

As described herein, one particular vesicle type contained within samples are exosomes. Exosomes are small, membrane-bound vesicles with a size of 40-150 nm (Pan et al, 1985; Trams et al, 1981). They are secreted by many different cell types, such as cancer cells, mesenchymal cells, thrombocytes (Kahlert and Kalluri, Exosomes in tumor microenvironment influence cancer progression and metastasis. J. Mol Med. (Berl), 91:431-437, 2013; Heijnen et al, Activated platelets release two types of membrane vesicles: microvesicles by surface shedding and exosomes derived from exocytosis of multivesicular bodies and alpha-granules. Blood, 94:3791-3799, 1999; Raposo et al, B lymphocytes secrete antigen-presenting vesicles. The Journal of Experimental Medicine, 183: 1161-1172, 1996), immune cells (Thery et al, Exosomes: composition, biogenesis and function. Nat. Rev. Immunol, 2:569-579, 2002), platelets (Janowska-Wieczorek et al, Microvesicles derived from activated platelets induce metastasis and angiogenesis in lung cancer. International Journal of Cancer, 113:752-760, 2005. Jazieh et al, The clinical utility of biomarkers in the management of pancreatic adenocarcinoma. Seminars in Radiation Oncology, 24:67-76, 2014), and endothelial cells (Hergenreider et al, Atheroprotective communication between endothelial cells and smooth muscle cells through miRNAs. Nature Cell Biology, 14:249-256, 2012). The first step in exosomes biogenesis involves the inward budding from the limiting membrane of late endosomes (Trajkovic et al, Ceramide triggers budding of exosome vesicles into multivesicular endosomes. Science, 319: 1244-1247, 2008). During this process, exosomes are packed with RNA molecules and proteins from the parental cell (Trams et al, Exfoliation of membrane ecto-enzymes in the form of micro-vesicles. Biochimica et Biophysica Acta, 645:63-70, 1981; Trajkovic Supra). After the release into the extracellular space, tumor-derived exosomes can transfer proteins and RNAs with oncogenic activity to recipient cells (Grange et al, Microvesicles released from human renal cancer stem cells stimulate angiogenesis and formation of lung premetastatic niche. Cancer Research, 71:5346-5356, 2011; Peinado et al, Melanoma exosomes educate bone marrow progenitor cells toward a pro-metastatic phenotype through MET. Nature Medicine, 18:883-891, 2012). Because exosomes are very stable under different conditions, they can protect their biological cargo against degradation and denaturation in the extracellular environment (Taylor and Gercel-Taylor, Exosomes/microvesicles: mediators of cancer-associated immunosuppressive microenvironments. Seminars in Immunopathology, 33:441-454, 2011). Genomic DNA in circulation is mainly contained in exosomes (Kahlert et al, Identification of double-stranded genomic DNA spanning all chromosomes with mutated KRAS and p53 DNA in the serum exosomes of patients with pancreatic cancer. The Journal of Biological Chemistry, 289:3869-3875, 2014). Exosomes from astrocytes and glioblastoma cells carry mitochondrial DNA (Guescini et al, C2C12 myoblasts release micro-vesicles containing DNA and proteins involved in signal transduction. Experimental Cell Research, 316: 1977-1984, 2010). Furthermore, it has been shown that exosomes from glioblastoma cell lines contain small amounts of single-stranded DNA as well as high levels of transposable elements (Balaj et al., Tumour microvesicles contain retrotransposon elements and amplified oncogene sequences. Nature Communications, 2: 180, 2011).

Exosomes are found in all body fluids of cancer patients, such as serum, saliva, cerebrospinal fluid, bone marrow aspirates, eye exudate/tears, and ascites (Peinado Supra; Lau et al, Role of Pancreatic Cancer-derived Exosomes in Salivary Biomarker Development. The Journal of Biological Chemistry, 288:26888-26897, 2013; Choi et al, Proteomic analysis of microvesicles derived from human colorectal cancer ascites. Proteomics, 11:2745-2751, 2011). As such, exosomes are promising diagnostic and predictive biomarkers in cancer. However, genetic profiling studies on circulating DNA from cancer patients are confounded by the fact that the isolated DNA represents all cells of the body, thus making mutation and genetic defects challenging (Murtaza et al, Non-invasive analysis of acquired resistance to cancer therapy by sequencing of plasma DNA. Nature, 497; 108-112, 2013; Yong, Cancer biomarkers: Written in blood. Nature, 511:524-526, 2014; Kirk, Breast cancer: Circulating tumour DNA the better of the blood biomarkers. Nature Reviews, Clinical Oncology, 10:247, 2013; Crowley et al, Liquid biopsy: monitoring cancer-genetics in the blood. Nature Reviews, Clinical Oncology, 10:472-484, 2013).

Several exosomes markers have been proposed and include members of the tetraspanin family (CD9, CD63, CD81), members of the endosomal sorting complexes required for transport (ESCRT; TSG101, Alix), and heat shock proteins (Hsp60, Hsp70, Hsp90) (Taylor and Gercel-Taylor, Supra). Epithelial tumor cells secrete exosomes carrying the epithelial cell adhesion molecule (EpCAM) (Taylor and Gercel-Taylor, Supra; Silva et al, Analysis of exosome release and its prognostic value in human colorectal cancer. Genes, Chromosomes & Cancer, 51:409-418, 2012; Runz et al., Malignant ascites-derived exosomes of ovarian carcinoma patients contain CD24 and EpCAM. Gynecologic Oncology, 107:563-571, 2007). Melanoma-derived exosomes contain the tumor-associated antigen Mart-1 and tyrosinase-related protein-2 (TYRP2) (Peinado, Supra; Mears et al, Proteomic analysis of melanoma-derived exosomes by two-dimensional polyacrylamide gel electrophoresis and mass spectrometry. Proteomics, 4:4019-4031, 2004; Andre et al, Malignant effusions and immunogenic tumour-derived exosomes. Lancet, 360:295-305, 2002). Exosomes from gastric cancer, breast cancer, and pancreatic cancer carry members of the human epidermal growth factor receptor (HER) family (Adamczyk et al, Characterization of soluble and exosomal forms of the EGFR released from pancreatic cancer cells. Life Sciences, 89:304-312, 2011; Baran et al, Circulating tumour-derived microvesicles in plasma of gastric cancer patients. Cancer Immunology, Immunotherapy: CII, 59:841-850, 2010; Ciravolo et al, Potential role of HER2-overexpressing exosomes in countering trastuzumab-based therapy. Journal of Cellular Physiology, 227:658-667, 2012).

The terms “microvesicle” and “exosome,” as used herein, refer to a membranous particle, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. In one embodiment, the sample is a blood sample, including, for example, whole blood or any fraction or component thereof. A blood sample suitable for use with the described technology may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one embodiment, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One exosome isolation method is differential centrifugation from body fluids or cell culture supernatants. Exemplary methods for isolation of exosomes are described in (Losche et al, Platelet-derived microvesicles transfer tissue factor to monocytes but not to neutrophils, Platelets, 15: 109-115, 2004; Mesri and Altieri, Endothelial cell activation by leukocyte microparticles, J. Immunol, 161:4382-4387, 1998; Morel et al, Cellular microparticles: a disseminated storage pool of bioactive vascular effectors, Curr. Opin. Hematol, 11: 156-164, 2004). Alternatively, exosomes may also be isolated via flow cytometry as described in (Combes et al., A new flow cytometry method of platelet-derived microvesicle quantitation in plasma, Thromb. Haemost., 77:220, 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

ii. Antibodies

As described herein, in certain embodiments antibodies are used as binding or capture agents. For example, in certain embodiments, an antibody (e.g., an anti-GLPC1) provided on a surface of a substrate as described herein can be used to capture exosomes wherein GLPC1 is present on the surface of the exosome. In other embodiments, an anti-GLPC3 provided on a surface can be used to capture exosomes comprising GLPC3.

An antibody fragment, e.g., functional fragment, is a portion of an antibody, e.g., F(ab′)2, F(ab)2, Fab′, Fab, domain antibody (dAb), variable fragment (Fv), or single chain variable fragment (scFv). A functional antibody fragment binds with the same antigen that is recognized by the intact antibody. For example, an anti-insulin monoclonal antibody fragment binds to insulin. The term “antibody fragment” or “functional fragment” also includes isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Antibody fragments include functional fragments and are encompassed by the terms “antibody” or “antibody molecule.”

Exemplary antibody molecules include full length antibodies and antibody fragments, e.g., dAb (domain antibody), single chain, Fab, Fab′, and F(ab′)2 fragments, and single chain variable fragments (scFvs).

A scFv polypeptide molecule is a covalently linked variable heavy chain (VH)::variable light chain (VL) heterodimer, which can be expressed from a gene fusion including VH and VL encoding genes linked by a peptide-encoding linker. See, e.g., Huston et al. (1988) Proc Nat Acad Sci USA 85(16):5879-5883. The N- to C-terminal orientation of the VH and VL domains can be in either orientation, e.g., VH-VL or VL-VH. Large naïve human scFv libraries have been created to provide a source of rearranged antibody genes against a variety of target molecules. To isolate disease-specific antibodies, libraries can be constructed from individuals with certain diseases. See, e.g., Barbas et al., Proc. Natl. Acad. Sci. USA 89:9339-43 (1992); and Zebedee et al., Proc. Natl. Acad. Sci. USA 89:3175-79 (1992).

Also provided herein are antibody fusion proteins, e.g., recombinantly produced antigen-binding molecules in which one or more of the same or different single-chain antibody or antibody fragment segments with the same or different specificities are linked. Valency of an antibody, e.g., fusion antibody protein, indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind, i.e., monospecific, bispecific, trispecific, multispecific. For example, a natural antibody, e.g., an IgG, is bivalent because it has two binding arms but is monospecific because it binds to one epitope. Monospecific, multivalent antibodies, e.g., antibody fusion proteins, have more than one binding site for an epitope but only bind with one epitope. The fusion protein can comprise a single antibody component, a multivalent or multispecific combination of different antibody components or multiple copies of the same antibody component. The fusion protein can additionally comprise an antibody or an antibody fragment and a therapeutic agent. Examples of therapeutic agents suitable for such fusion proteins include immunomodulators and toxins. Exemplary toxins include but are not limited to ribonuclease (RNase), e.g., recombinant RNase, Diphtheria toxin, Pseudomonas exotoxin, monomethyl auristatin E, or mertansine. Additional exemplary toxins are described herein. In embodiments, the antibody molecule (e.g., antibody or functional fragment thereof) and the therapeutic agent (e.g., toxin) are encoded by a single nucleic acid molecule. In embodiments, the antibody molecule (e.g., antibody or functional fragment thereof) and the therapeutic agent (e.g., toxin) are disposed on the same polypeptide. In other embodiments, the antibody molecule (e.g., antibody or functional fragment thereof) and the therapeutic agent (e.g., toxin) are encoded by separate nucleic acid molecules. In embodiments, the antibody molecule (e.g., antibody or functional fragment thereof) and the therapeutic agent (e.g., toxin) are disposed on separate polypeptides. A variety of protein or peptide effectors may be incorporated into a fusion protein. Conjugates/fusions to toxins are discussed further below.

Humanized, Chimeric, or Fully Human Antibody Molecules

Also provided herein are humanized, chimeric, or fully human antibody molecules, e.g., full length antibodies, antibody fragments, antibody or antibody fragment fusions, or antibody or antibody fragment conjugates.

A humanized antibody is a recombinant protein in which the complementarity determining regions (CDRs) from an antibody from one species; e.g., a rodent (e.g., rat or mice) antibody, are transferred from the heavy and light variable chains of the rodent antibody into human heavy and light variable domains. The constant domains of the antibody molecule are derived from those of a human antibody.

Methods for humanizing non-human antibodies have been described in the art. In embodiments, a humanized antibody has one or more amino acid residues introduced into it from a source which is non-human. These non-human amino acid residues are often referred to as “import” residues, which are typically taken from an “import” variable domain. Humanization can be performed following the method of Winter and co-workers (Jones et al., Nature, 321: 522-525 (1986); Reichmann et al., Nature, 332: 323-327 (1988); Verhoeyen et al., Science, 239: 1534-1536 (1988)), e.g., by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In embodiments, humanized antibodies are antibodies in which some hypervariable region residues and possibly some FR residues are substituted by residues from analogous sites in rodent antibodies.

The choice of human variable domains, both light and heavy, to be used in making the humanized antibodies can play a role in reducing antigenicity. In some embodiments, according to the so called “best-fit” method, the sequence of the variable domain of a rodent antibody is screened against the entire library of known human variable-domain sequences. The human sequence closest to that of the rodent is then accepted as the human framework region (FR) for the humanized antibody (Suns et al., J. Immunol., 151: 2296 (1993); Chothia et al., J. Mol. Biol, 196: 901 (1987)). In embodiments, another method uses a particular framework region derived from the consensus sequence of all human antibodies of a particular subgroup of light or heavy chains. The same framework may be used for several different humanized antibodies (Carter et al., Proc. Natl. Acad. Sci. USA, 89: 4285 (1992); Presta et al., J. Immunol., 151: 2623 (1993)).

In embodiments, antibodies are humanized with retention of high affinity for the antigen and other favorable biological properties. To achieve this goal, in certain embodiments, humanized antibodies are prepared by a process of analysis of the parental sequences and various conceptual humanized products using three-dimensional models of the parental and humanized sequences. Three-dimensional immunoglobulin models are commonly available. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as preserved or increased affinity for the target antigen, is achieved. In general, the hypervariable region residues are directly and most substantially involved in influencing antigen binding.

In embodiments, a humanized antibody molecule, e.g., humanized antibody molecule described herein, comprises one or more non-human (e.g., mouse) CDRs and comprises human framework and constant regions (e.g., framework and constant regions from a human immunoglobulin, e.g., IgG1, IgG2, IgG3, or IgG4).

Antibody Production

Various procedures known within the art may be used for the production of antibody molecules, e.g., antibodies or functional fragments thereof, directed against a protein or peptide of the described technology, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

In some embodiments, an autoantigen (e.g., islet autoantigen, e.g., an islet autoantigen described herein, e.g., insulin), a B cell (e.g., autoantigen-specific B cell, e.g., insulin-specific B cell), or an autoantigen::B cell receptor (BCR) complex (e.g., insulin::BCR complex), can be utilized as an immunogen in the generation of antibody molecules that immunospecifically bind these protein components.

Antibody molecules can be purified by well-known techniques, such as affinity chromatography using protein A or protein G, e.g., which provide the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

In embodiments, the immunizing agent includes the protein antigen, a fragment thereof or a fusion protein thereof. In accordance with the compositions and methods described herein, the immunizing agent comprises an autoantigen, e.g., islet autoantigen, e.g., islet autoantigen described herein, e.g., insulin. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. In embodiments, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. Exemplary immortalized cell lines are murine myeloma lines, which can be obtained, for instance, from the Salk Institute Cell Distribution Center, San Diego, Calif. And the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (MA), enzyme-linked immunoabsorbent assay (ELISA), flow cytometry/FACS, or surface plasmon resonance. Such techniques and assays are known in the art. The binding affinity of the monoclonal antibody can, for example, be determined by the Scatchard analysis of Munson and Pollard, Anal. Biochem., 107:220 (1980). In embodiments, in therapeutic applications of monoclonal antibodies, it can be important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods. DNA encoding the monoclonal antibodies described herein can be readily isolated and sequenced using conventional procedures (e.g., by using oligonucleotide probes that are capable of binding specifically to genes encoding the heavy and light chains of murine antibodies). In embodiments, hybridoma cells serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences or by covalently joining to the immunoglobulin coding sequence all or part of the coding sequence for a non-immunoglobulin polypeptide. Such a non-immunoglobulin polypeptide can be substituted for the constant domains of an antibody of the described technology, or can be substituted for the variable domains of one antigen-combining site of an antibody of the described technology to create a chimeric bivalent antibody.

Fully human antibodies are antibody molecules in which the entire sequence of both the light chain and the heavy chain, including the CDRs, arise from human genes. Such antibodies are termed “human” antibodies, or “fully human” antibodies herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: Monoclonal Antibodies And Cancer Therapy, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies And Cancer Therapy, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice, in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An embodiment of such a nonhuman animal is a mouse, and is termed the Xenomouse™. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs or fragments of antibodies such as, for example, single chain Fv (scFv) molecules.

An exemplary method for producing an antibody described herein. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain. In an embodiment, a method for identifying a clinically relevant epitope on an immunogen, and a correlative method for selecting an antibody that binds immunospecifically to the relevant epitope with high affinity.

Vectors

An antibody molecule can be expressed by a vector containing a DNA segment encoding the antibody molecule, e.g., antibody molecule described herein.

These can include vectors, liposomes, naked DNA, adjuvant-assisted DNA, gene gun, catheters, etc. Vectors include chemical conjugates such as described in WO 93/64701, which has targeting moiety (e.g. A ligand to a cellular surface receptor), and a nucleic acid binding moiety (e.g., polylysine), viral vector (e.g. A DNA or RNA viral vector), fusion proteins which is a fusion protein containing a target moiety (e.g. An antibody specific for a target cell) and a nucleic acid binding moiety (e.g. A protamine), plasmids, phage, etc. The vectors can be chromosomal, non-chromosomal or synthetic.

Exemplary vectors include viral vectors, fusion proteins and chemical conjugates. Retroviral vectors include moloney murine leukemia viruses. In embodiments, the viral vector is a DNA viral vector. Exemplary DNA vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci. USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short term expression of the nucleic acid. In embodiments, adenovirus vectors, adeno-associated virus vectors and herpes simplex virus (HSV) vectors are used for introducing the nucleic acid into cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. Adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express antibody molecules, e.g., antibody molecules described herein. Techniques can be adapted for the production of single-chain antibodies specific to an antigenic protein of the described technology. In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

iii. Capture Agents

In certain embodiments as described herein, a capture agent is present on the top surface of a substrate as described herein (e.g., an optical substrate, e.g., a reflective substrate) such that the capture agent serves to bind to a target of interest. In certain embodiments, the target of interest is associated with vesicles such that the binding of the capture agent to the vesicle serves to indirectly bind the vesicle to the surface of the substrate. By way of illustration and not limitation, in certain embodiments, a capture agent may be an antibody specific, such as one specific to a cancer associated protein (e.g., specific to a member of the tetraspanin family e.g., an anti-CD 63 antibody, an anti-CD 81 antibody, an anti-CD 9 antibody, an anti-CD171 antibody).

Illustrative capture agents include, but are not limited to antibodies (including monoclonal antibodies, polyclonal antibodies, bispecific antibodies, or antigen-binding fragments thereof, and antibody fragment including, ScFv, F(ab), F(ab′)2, Fv), isotope labeled peptides, nucleic acid probes, DNA or RNA aptamers, as well as the use of click chemistry for target-guided synthesis (Lewis et al., Angewandte Chemie-International Edition, 41, 1053-, 2002; Manetsch et al., J. Am. Chem. Soc. 126, 12809-12818, 2004; Ramstrom et al., Nature Rev. Drug Discov. 1, 26-36, 2002), small molecule compounds, and polymers.

iv. Fluorescent Dyes

In certain embodiments, the disclosed systems and methods detect cargo markers using proteins (e.g., antibodies) labeled with fluorophores. Such fluorescent labeled proteins are referred to as “fluorescent cargo labels”. Fluorophores comprise fluorochromes, fluorochrome quencher molecules, any organic or inorganic dyes, metal chelates, or any fluorescent enzyme substrates, including protease activatable enzyme substrates. In certain embodiments, fluorophores comprise long chain carbophilic cyanines. In other embodiments, fluorophores comprise DiI, DiR, DiD, and the like. Fluorochromes comprise far red, and near infrared fluorochromes (NIRF). Fluorochromes include but are not limited to a carbocyanine and indocyanine fluorochromes. In certain embodiments, imaging agents comprise commercially available fluorochromes including, but not limited to Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); CF dyes (Sigma-Aldrich); phycoerithrin (PE); and ADS780WS, ADS830WS, and ADS832WS (American Dye Source) and Kodak X-SIGHT 650, Kodak X-SIGHT 691, Kodak X-SIGHT 751 (Carestream Health). In certain embodiments, the molecular weight of the fluorophore impacts labeling efficiency of molecular cargo inside the exosomes.

### G. COMPUTER SYSTEM AND NETWORK ENVIRONMENT

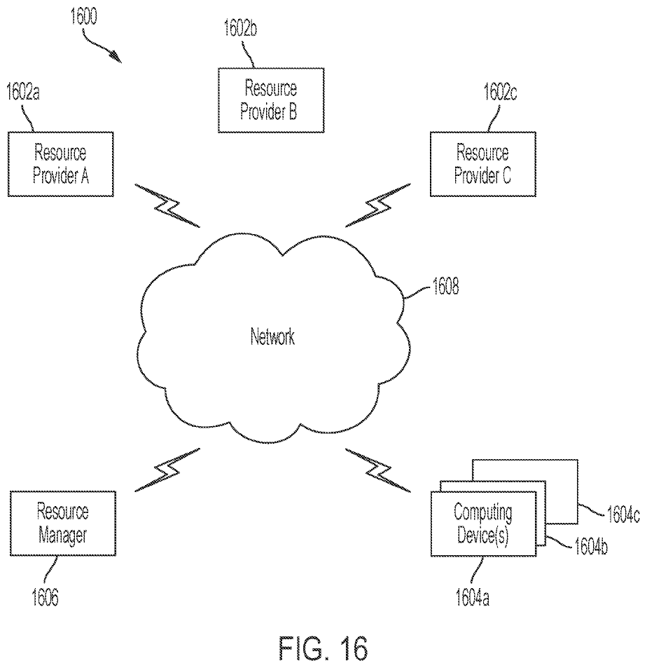

As shown in FIG. 16, an implementation of a network environment 1600 for use in providing the systems and methods described herein is shown and described. In brief overview, referring now to FIG. 16, a block diagram of an exemplary cloud computing environment 1600 is shown and described. The cloud computing environment 1600 may include one or more resource providers 1602a, 1602b, 1602c (collectively, 1602). Each resource provider 1602 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1602 may be connected to any other resource provider 1602 in the cloud computing environment 1600. In some implementations, the resource providers 1602 may be connected over a computer network 1608. Each resource provider 1602 may be connected to one or more computing device 1604a, 1604b, 1604c (collectively, 1604), over the computer network 1608.

The cloud computing environment 1600 may include a resource manager 1606. The resource manager 1606 may be connected to the resource providers 1602 and the computing devices 1604 over the computer network 1608. In some implementations, the resource manager 1606 may facilitate the provision of computing resources by one or more resource providers 1602 to one or more computing devices 1604. The resource manager 1606 may receive a request for a computing resource from a particular computing device 1604. The resource manager 1606 may identify one or more resource providers 1602 capable of providing the computing resource requested by the computing device 1604. The resource manager 1606 may select a resource provider 1602 to provide the computing resource. The resource manager 1606 may facilitate a connection between the resource provider 1602 and a particular computing device 1604. In some implementations, the resource manager 1606 may establish a connection between a particular resource provider 1602 and a particular computing device 1604. In some implementations, the resource manager 1606 may redirect a particular computing device 1604 to a particular resource provider 1602 with the requested computing resource.

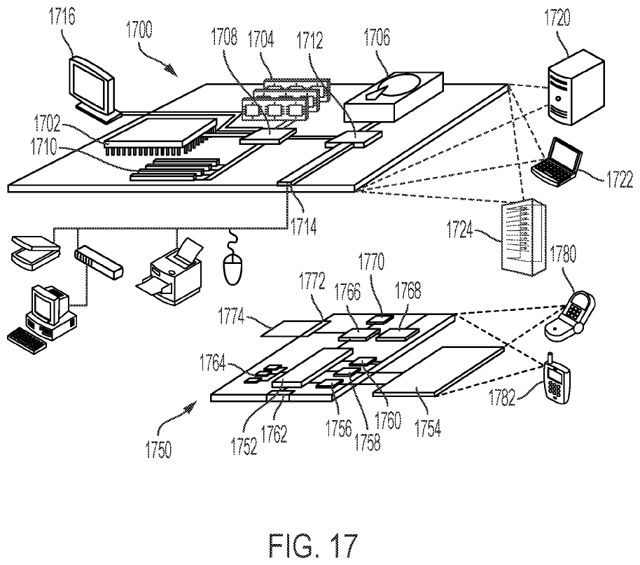

FIG. 17 shows an example of a computing device 1700 and a mobile computing device 1750 that can be used to implement the techniques described in this disclosure. The computing device 1700 is intended to represent various forms of digital computers, such as laptops, desktops, workstations, personal digital assistants, servers, blade servers, mainframes, and other appropriate computers. The mobile computing device 1750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1700 includes a processor 1702, a memory 1704, a storage device 1706, a high-speed interface 1708 connecting to the memory 1704 and multiple high-speed expansion ports 1710, and a low-speed interface 1712 connecting to a low-speed expansion port 1715 and the storage device 1706. Each of the processor 1702, the memory 1704, the storage device 1706, the high-speed interface 1708, the high-speed expansion ports 1710, and the low-speed interface 1712, are interconnected using various busses, and may be mounted on a common motherboard or in other manners as appropriate. The processor 1702 can process instructions for execution within the computing device 1700, including instructions stored in the memory 1704 or on the storage device 1706 to display graphical information for a GUI on an external input/output device, such as a display 1716 coupled to the high-speed interface 1708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1704 stores information within the computing device 1700. In some implementations, the memory 1704 is a volatile memory unit or units. In some implementations, the memory 1704 is a non-volatile memory unit or units. The memory 1704 may also be another form of computer-readable medium, such as a magnetic or optical disk.

The storage device 1706 is capable of providing mass storage for the computing device 1700. In some implementations, the storage device 1706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1702), perform one or more methods, such as those described above. The instructions can also be stored by one or more storage devices such as computer- or machine-readable mediums (for example, the memory 1704, the storage device 1706, or memory on the processor 1702).

The high-speed interface 1708 manages bandwidth-intensive operations for the computing device 1700, while the low-speed interface 1712 manages lower bandwidth-intensive operations. Such allocation of functions is an example only. In some implementations, the high-speed interface 1708 is coupled to the memory 1704, the display 1716 (e.g., through a graphics processor or accelerator), and to the high-speed expansion ports 1710, which may accept various expansion cards (not shown). In the implementation, the low-speed interface 1712 is coupled to the storage device 1706 and the low-speed expansion port 1714. The low-speed expansion port 1714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1700 may be implemented in a number of different forms, as shown in the figure. For example, it may be implemented as a standard server 1720, or multiple times in a group of such servers. In addition, it may be implemented in a personal computer such as a laptop computer 1722. It may also be implemented as part of a rack server system 1724. Alternatively, components from the computing device 1700 may be combined with other components in a mobile device (not shown), such as a mobile computing device 1750. Each of such devices may contain one or more of the computing device 1700 and the mobile computing device 1750, and an entire system may be made up of multiple computing devices communicating with each other.

The mobile computing device 1750 includes a processor 1752, a memory 1764, an input/output device such as a display 1754, a communication interface 1766, and a transceiver 1768, among other components. The mobile computing device 1750 may also be provided with a storage device, such as a micro-drive or other device, to provide additional storage. Each of the processor 1752, the memory 1764, the display 1754, the communication interface 1766, and the transceiver 1768, are interconnected using various buses, and several of the components may be mounted on a common motherboard or in other manners as appropriate.

The processor 1752 can execute instructions within the mobile computing device 1750, including instructions stored in the memory 1764. The processor 1752 may be implemented as a chipset of chips that include separate and multiple analog and digital processors. The processor 1752 may provide, for example, for coordination of the other components of the mobile computing device 1750, such as control of user interfaces, applications run by the mobile computing device 1750, and wireless communication by the mobile computing device 1750.

The processor 1752 may communicate with a user through a control interface 1758 and a display interface 1756 coupled to the display 1754. The display 1754 may be, for example, a TFT (Thin-Film-Transistor Liquid Crystal Display) display or an OLED (Organic Light Emitting Diode) display, or other appropriate display technology. The display interface 1756 may comprise appropriate circuitry for driving the display 1754 to present graphical and other information to a user. The control interface 1758 may receive commands from a user and convert them for submission to the processor 1752. In addition, an external interface 1762 may provide communication with the processor 1752, so as to facilitate near area communication of the mobile computing device 1750 with other devices. The external interface 1762 may provide, for example, for wired communication in some implementations, or for wireless communication in other implementations, and multiple interfaces may also be used.

The memory 1764 stores information within the mobile computing device 1750. The memory 1764 can be implemented as one or more of a computer-readable medium or media, a volatile memory unit or units, or a non-volatile memory unit or units. An expansion memory 1774 may also be provided and connected to the mobile computing device 1750 through an expansion interface 1772, which may include, for example, a SIMM (Single In Line Memory Module) card interface. The expansion memory 1774 may provide extra storage space for the mobile computing device 1750, or may also store applications or other information for the mobile computing device 1750. Specifically, the expansion memory 1774 may include instructions to carry out or supplement the processes described above, and may include secure information also. Thus, for example, the expansion memory 1774 may be provide as a security module for the mobile computing device 1750, and may be programmed with instructions that permit secure use of the mobile computing device 1750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1752), perform one or more methods, such as those described above. The instructions can also be stored by one or more storage devices, such as one or more computer- or machine-readable mediums (for example, the memory 1764, the expansion memory 1774, or memory on the processor 1752). In some implementations, the instructions can be received in a propagated signal, for example, over the transceiver 1768 or the external interface 1762.

The mobile computing device 1750 may communicate wirelessly through the communication interface 1766, which may include digital signal processing circuitry where necessary. The communication interface 1766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1768 using a radio-frequency. In addition, short-range communication may occur, such as using a Bluetooth®, Wi-Fi™, or other such transceiver (not shown). In addition, a GPS (Global Positioning System) receiver module 1770 may provide additional navigation- and location-related wireless data to the mobile computing device 1750, which may be used as appropriate by applications running on the mobile computing device 1750.

The mobile computing device 1750 may also communicate audibly using an audio codec 1760, which may receive spoken information from a user and convert it to usable digital information. The audio codec 1760 may likewise generate audible sound for a user, such as through a speaker, e.g., in a handset of the mobile computing device 1750. Such sound may include sound from voice telephone calls, may include recorded sound (e.g., voice messages, music files, etc.) and may also include sound generated by applications operating on the mobile computing device 1750.

The mobile computing device 1750 may be implemented in a number of different forms, as shown in the figure. For example, it may be implemented as a cellular telephone 1780. It may also be implemented as part of a smart-phone 1782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, any modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. Described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the described technology that consist essentially of, or consist of, the recited components, and that there are processes and methods according to the described technology that consist essentially of, or consist of, the recited processing steps.

It should be understood that the order of steps or order for performing certain action is immaterial so long as the described technology remains operable. Moreover, two or more steps or actions may be conducted simultaneously.

While the described technology has been particularly shown and described with reference to specific embodiments, it should be understood by those skilled in the art that various changes in form and detail may be made therein without departing from the spirit and scope of the described technology as defined by the appended claims.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of isolating, labeling, and imaging vesicles and their biomolecular cargo, the method comprising:
(a) contacting a top surface of a substrate with a sample comprising the vesicles, thereby capturing one or more vesicles present in the sample;
(b) contacting vesicles with a permeabilization solution comprising a permeabilization agent, thereby permeabilizing the vesicles;
(c) following step (b), contacting vesicles with one or more fluorescent cargo labels, wherein each fluorescent cargo label is: (i) specific to a particular biomolecule of interest of one or more biomolecules of interest and (ii) comprises a particular fluorescent species, thereby labeling the biomolecular cargo within the vesicles;
(d) directing excitation light toward the top surface of the substrate, thereby exciting the one or more fluorescent cargo labels with which the vesicles are labeled;
(e) detecting, with one or more detectors, fluorescent light emitted from the one or more fluorescent cargo labels as a result of excitation by the excitation light; and
(g) using the detected fluorescent light to detect and/or quantify, at least a portion of the one or more biomolecules of interest present within the vesicles.

2. The method of claim 1, comprising performing step (b) and (c) following step (a), so as to permeabilize and label the vesicles after they are captured onto the top surface of the substrate.

3. The method of claim 1, comprising performing step (b) and step (c) before step (a), so as to permeabilize and label the vesicles before they are captured onto the top surface of the substrate.

4. The method of any one of the preceding claims, wherein the vesicles are less than or approximately equal to 1 micron in diameter.

5. The method of any one of the preceding claims, wherein the vesicles are extracellular vesicles.

6. The method of claim 5, wherein the extracellular vesicles are exosomes.

7. The method of any one of the preceding claims, comprising contacting the vesicles with a crosslinking agent, thereby fixing the vesicles.

8. The method of claim 7, comprising incubating the vesicles with the crosslinking agent for a duration selected to avoid over-fixation of the vesicles.

9. The method of either of claim 7 or 8, wherein a concentration of the crosslinking agent is selected to avoid over-fixation of the vesicles.

10. The method of any one of the preceding claims, wherein step (b) comprising incubating the vesicles with the permeabilization agent for a duration selected to maintain integrity of a membrane of the vesicles.

11. The method of any one of the preceding claims, wherein a concentration of the permeabilization agent in the permeabilization solution is selected to maintain integrity of a membrane of the vesicles.

12. The method of any one of the preceding claims, wherein the top surface of the substrate comprises one or more capture agents, each capture agent specific to a particular target agent of one or more target agents associated with at least a portion of the vesicles.

13. The method of claim 12, wherein the particular target agent to which each of at least a portion of the one or more capture agents are specific is a surface marker associated with a particular disease and/or condition.

14. The method of either of claim 12 or 13, wherein: (i) the one or more capture agents comprise an antibody specific to a cancer associated protein and/or (ii) the one or more target agents comprise one or more cancer associated proteins.

15. The method of any one of the preceding claims, wherein the one or more biomolecules of interest comprise one or more proteins.

16. The method of any one of the preceding claims, wherein the one or more biomolecules of interest comprise one or more nucleic acids.

17. The method of any one of the preceding claims, wherein step (c) comprises contacting the vesicles with one or more cargo labeling solutions, each comprising at least one of the one or more fluorescent cargo labels and a blocking agent.

18. The method of any one of the preceding claims, wherein a concentration of each of at least a portion of the fluorescent cargo labels is about 1 microgram per milliliter or less.

19. The method of any one of the preceding claims, comprising:
contacting the vesicles with a fluorescent vesicle detection agent specific to a particular target agent associated with at least a portion of the vesicles, thereby labeling the vesicles with the fluorescent vesicle detection agent;
at step (d), exciting the fluorescent vesicle detection agent; and
at step (e), detecting, with the one or more detectors, fluorescent light emitted from the fluorescent vesicle detection agent as a result of excitation by the excitation light.

20. The method of any one of the preceding claims, wherein step (e) comprises imaging the top surface of the substrate at one or more fluorescent wavelengths, each corresponding to an emission wavelength of a fluorescent cargo label, thereby obtaining one or more fluorescent images, each associated with a particular fluorescent cargo label and the particular biomolecule of interest to which the particular fluorescent cargo label is specific.

21. The method of claim 20, wherein step (g) comprises:
receiving and/or accessing, by a processor of a computing device, the one or more fluorescent images;
identifying, by the processor, within each of at least a portion of the one or more fluorescent images, a plurality of discrete points of fluorescent emission, each determined to originate from within a vesicle; and
using the discrete points of fluorescent emission to detect and/or quantify, by the processor, the portion of the one or more particular biomolecules of interest.

22. The method of any one of the preceding claims, comprising:
(h) directing illumination light toward the top surface of the substrate, thereby illuminating the captured vesicles along with the substrate; and
(i) detecting, with the one or more detectors, a label-free signal corresponding to a portion of the illumination light that is (A) scattered by the vesicles and/or (B) reflected by the substrate.

23. The method of any one of claims 20 to 22, wherein the imaging is performed using a high magnification objective lens having sufficiently high magnification and resolution to detect the fluorescent light emitted from the fluorescently labeled vesicles situated on the top surface of the substrate.

24. The method of claim 23, wherein the high magnification objective lens has a magnification ranging from about 4× to about 100×.

25. The method of either of claim 23 or 24, wherein the high magnification objective lens has a numerical aperture ranging from about 0.1 and about 1.3.

26. The method of any one of claims 22 to 25, wherein step (g) comprises using the detected label-free signal along with the detected fluorescent light to detect and/or quantify the portion of the one or more biomolecules of interest.

27. The method of any one of claims 22 to 26, wherein step (i) comprises imaging the top surface of the substrate at one or more wavelengths of the illumination light, thereby obtaining one or more label-free images, each associated with a particular illumination wavelength.

28. The method of claim 27, wherein step (g) comprises:
receiving and/or accessing, by a processor of a computing device, the one or more label-free images;
identifying, by the processor, a plurality of vesicle locations on the top surface of the substrate using the one or more label-free images; and
using the identified vesicle locations to detect and/or quantify the portion of the one or more particular biomolecules of interest.

29. The method of any one of the preceding claims, wherein the substrate is a reflective substrate comprising an optical interference coating comprising a stack of one or more layers, wherein a thickness and/or material of each of the one or more layers in the stack is such that:
(A) excitation of and/or emission of fluorescent light from one or more of the fluorescent cargo labels is enhanced, and/or
(B) a label free signal, obtained by detection of light scattered by the vesicles in response to illumination by illumination light, is enhanced.

30. The method of claim 29, wherein the reflective substrate has a reflectance greater than 25% at one or more particular wavelengths.

31. A method of isolating, permeabilizing, and labeling vesicles and their biomolecular cargo, the method comprising:
(a) contacting a surface of a substrate with a sample comprising the vesicles, thereby capturing one or more vesicles present in the sample;
(b) contacting the vesicles with a permeabilization solution comprising a permeabilization agent, thereby permeabilizing the captured vesicles, wherein a duration with which the vesicles are incubated with the permeabilization solution and/or a concentration of the permeabilization agent in the permeabilization solution is selected to maintain integrity of a membrane of the vesicles; and
(c) following step (b), contacting the vesicles with one or more fluorescent cargo labels, wherein each fluorescent cargo label is: (i) specific to a particular biomolecule of interest of one or more biomolecules of interest within the vesicles and (ii) comprises a particular fluorescent species, thereby labeling the biomolecular cargo within the vesicles.

32. The method of claim 31, comprising performing step (b) and (c) following step (a), so as to permeabilize and label the vesicles after they are captured.

33. The method of claim 31, comprising performing step (b) and step (c) before step (a), so as to permeabilize and label the vesicles before they are captured.

34. The method of any one of claims 31 to 33, wherein the vesicles are less than or approximately equal to 1 micron in diameter.

35. The method of any one of claims 31 to 34, wherein the vesicles are extracellular vesicles.

36. The method of claim 35, wherein the extracellular vesicles are exosomes.

37. The method of any one of claims 31 to 36, comprising contacting the vesicles with a crosslinking agent, thereby fixing the vesicles.

38. The method of claim 37, comprising incubating the vesicles with the crosslinking agent for a duration selected to avoid over-fixation of the vesicles.

39. The method of either of claim 37 or 38, wherein a concentration of the crosslinking agent is selected to avoid over-fixation of the vesicles.

40. The method of any one of claims 31 to 39, wherein step (b) comprising incubating the vesicles with the permeabilization agent for a duration selected to maintain integrity of a membrane of the vesicles.

41. The method of any one of claims 31 to 40, wherein a concentration of the permeabilization agent in the permeabilization solution is selected to maintain integrity of a membrane of the vesicles.

42. The method of any one of claims 31 to 41, wherein the surface of the substrate comprises one or more capture agents, each capture agent specific to a particular target agent of one or more target agents associated with at least a portion of the vesicles.

43. The method of claim 42, wherein the particular target agent to which each of at least a portion of the one or more capture agents are specific is a surface marker associated with a particular disease and/or condition.

44. The method of either of claim 42 or 43, wherein: (i) the one or more capture agents comprise an antibody specific to a cancer associated protein and/or (ii) the one or more target agents comprise one or more cancer associated proteins.

45. The method of any one of claims 31 to 44, wherein the one or more biomolecules of interest comprise one or more proteins.

46. The method of any one of claims 31 to 45, wherein the one or more biomolecules of interest comprise one or more biomolecules of interest comprise one or more nucleic acids.

47. The method of any one of claims 31 to 46, wherein step (c) comprises contacting the vesicles with one or more cargo labeling solutions, each comprising at least one of the one or more fluorescent cargo labels and a blocking agent.

48. The method of any one of claims 31 to 47, wherein a concentration of each of at least a portion of the fluorescent cargo labels is about 1 microgram per milliliter or less.

49. The method of any one of claims 31 to 48, comprising:
contacting the vesicles with a fluorescent vesicle detection agent specific to a particular target agent associated with at least a portion of the vesicles, thereby labeling the vesicles with the fluorescent vesicle detection agent.

50. A kit for isolating, permeabilizing, and labeling vesicles and their biomolecular cargo, the kit comprising:
(a) a pre-mixed permeabilization solution comprising a permeabilization agent; and
(b) one or more pre-mixed cargo labeling solutions, each comprising one or more fluorescent cargo labels, wherein each fluorescent cargo label is (i) specific to a particular biomolecule of interest of one or more biomolecules of interest and (ii) comprises a particular fluorescent species.

51. The kit of claim 50, further comprising one or more pre-mixed capture agent solutions, each comprising one or more capture agents, wherein each capture agent is specific to a particular target agent associated with at least a portion of the vesicles.

52. The kit of claim 50, further comprising a pre-spotted substrate, wherein the pre-spotted substrate comprises one or more capture agent spots, each of the one or more capture agent spots comprising a particular capture agent specific to a particular target agent associated with at least a portion of the vesicles.

53. The kit of either of claim 51 or 52, wherein the particular target agent to which each of at least a portion of the one or more capture agents are specific is a surface marker associated with a particular disease and/or condition.

54. The kit of any one of claims 50 to 53, wherein: (i) the one or more capture agents comprise an antibody specific to a cancer associated protein and/or (ii) the one or more target agents comprise one or more cancer associated proteins.

55. The kit of any one of claims 50 to 54, comprising a fixing solution comprising a crosslinking agent.

56. The kit of claim 55, wherein a concentration of the crosslinking agent in the fixing solution is selected to avoid over-fixation of the vesicles.

57. The kit of any one of claims 50 to 56, wherein a concentration of the permeabilization agent in the permeabilization solution is selected to maintain integrity of a membrane of the vesicles.

58. The kit of any one of claims 50 to 57, wherein the one or more biomolecules of interest comprise one or more proteins.

59. The kit of any one of claims 50 to 58 wherein the one or more biomolecules of interest comprise one or more biomolecules of interest comprise one or more nucleic acids.

60. The kit of any one of claims 50 to 59, wherein a concentration of each of at least a portion of the fluorescent cargo labels is about 1 microgram per milliliter or less.

61. The kit of any one of claims 50 to 60, comprising a vesicle detection solution comprising a fluorescent vesicle detection agent specific to a particular target agent.

62. The kit of any one of claims 50 to 61, comprising a reflective substrate comprising an optical interference coating comprising a stack of one or more layers, wherein a thickness and/or material of each of the one or more layers in the stack is such that:
(A) excitation of and/or emission of fluorescent light from one or more of the fluorescent cargo labels is enhanced, and/or
(B) a label free signal, obtained by detection of light scattered by the vesicles in response to illumination by illumination light, is enhanced.

63. The kit of claim 62, wherein the reflective substrate has a reflectance greater than 25% at one or more particular wavelengths.

64. A system for isolating, labeling, and imaging vesicles and their biomolecular cargo, the system comprising:
(a) a kit for isolating, permeabilizing, and labeling vesicles and their biomolecular cargo;
(b) a mount for holding a substrate;
(c) one or more excitation light sources aligned with respect to the mount so as to and direct excitation light toward a top surface of the substrate, so as to provide for excitation of one or more fluorescently labeled vesicles situated on the top surface of the substrate;
(d) one or more detectors aligned with respect to the mount and operable to detect fluorescent light emitted from the fluorescently labeled vesicles situated on the top surface of the substrate;
(e) a processor of a computing device; and
(f) a memory having instructions stored thereon, wherein the instructions, when executed by the processor, cause the processor to:
receive and/or access data corresponding to the detected fluorescent light; and
use the data corresponding to the detected fluorescent light to detect and/or quantify the biomolecular cargo of the vesicles.

65. The system of claim 64, wherein the one or more detectors are each aligned with respect a high magnification objective lens having sufficiently high magnification and resolution to detect the fluorescent light emitted from the fluorescently labeled vesicles situated on the top surface of the substrate.

66. The system of claim 65 wherein the high magnification objective lens has a magnification ranging from about 4× to about 100×.

67. The system of claim 65 or 66, wherein the high magnification objective lens has a numerical aperture ranging from about 0.1 and about 1.3.

68. The system of any one of claims 64 to 67, wherein the kit comprises:
(A) a pre-mixed permeabilization solution comprising a permeabilization agent; and
(B) one or more pre-mixed cargo labeling solutions, each comprising one or more fluorescent cargo labels, wherein each fluorescent cargo label is (i) specific to a particular biomolecule of interest of one or more biomolecules of interest and (ii) comprises a particular fluorescent species.

69. The system of claim 68, wherein a concentration of the permeabilization agent in the permeabilization solution is selected to maintain integrity of a membrane of the vesicles.

70. The system of any one of claims 68 to 69, wherein the one or more biomolecules of interest comprise one or more proteins.

71. The system of any one of claims 68 to 70, wherein the one or more biomolecules of interest comprise one or more biomolecules of interest comprise one or more nucleic acids.

72. The system of any one of claims 68 to 71, wherein a concentration of each of at least a portion of the fluorescent cargo labels is about 1 microgram per milliliter or less.

73. The system of any one of claims 64 to 72, wherein the kit further comprises one or more pre-mixed capture agent solutions, each comprising one or more capture agents, wherein each capture agent is specific to a particular target agent associated with at least a portion of the vesicles.

74. The system of any one of claims 64 to 73, wherein the kit further comprises a pre-spotted substrate, wherein the pre-spotted substrate comprises one or more capture agent spots, each of the one or more capture agent spot comprising a particular capture agent specific to a particular target agent associated with at least a portion of the vesicles.

75. The system of either claim 73 or 74, wherein the particular target agent to which each of at least a portion of the one or more capture agents are specific is a surface marker associated with a particular disease and/or condition.

76. The system of any one of claims 73 to 75, wherein: (i) the one or more capture agents comprise an antibody specific to a cancer associated protein and/or (ii) the one or more target agents comprise one or more cancer associated proteins.

77. The system of any one of claims 73 to 76, wherein the kit comprises a fixing solution comprising a crosslinking agent for fixing the vesicles.

78. The system claim 77, wherein a concentration of the crosslinking agent in the fixing solution is selected to avoid over-fixation of the vesicles.

79. The system of any one of claims 64 to 78, wherein the kit comprises a vesicle detection solution comprising a fluorescent vesicle detection agent specific to a particular target agent associated with at least a portion of the vesicles.

80. The system of any one of claims 64 to 79, comprising a reflective substrate comprising an optical interference coating comprising a stack of one or more layers, wherein a thickness and/or material of each of the one or more layers in the stack is such that:
(A) excitation of and/or emission of fluorescent light from one or more of the fluorescent cargo labels is enhanced, and/or
(B) a label free signal, obtained by detection of light scattered by the vesicles in response to illumination by illumination light, is enhanced.

81. The system of claim 80, wherein the reflective substrate has a reflectance greater than 25%, at one or more particular wavelengths.
